(19) United States
(12) Patent Application Publication
(10) Pub. No.: US 2025/0376716 A1
(43) Pub. Date: Dec. 11, 2025

(54) METHOD FOR DETECTING ANALYTES AT SITES OF OPTICALLY NON-RESOLVABLE DISTANCES

(71) Applicant: Nautilus Subsidiary, Inc., Seattle, WA (US)

(72) Inventors: Ezra VAN GELDER, Palo Alto, CA (US); Pierre INDERMUHLE, Berkeley, CA (US); David STERN, Mountain View, CA (US); Meysam Rezaei BARMI, San Pedro, CA (US); Michael Augusto DARCY, Fremont, CA (US)

(21) Appl. No.: 18/876,169

(22) PCT Filed: Jun. 21, 2023
(86) PCT No.: PCT/US2023/068798

§ 371 Date: Dec. 17, 2024

Related U.S. Application Data
(60) Provisional application No. 63/354,169, filed on Jun. 21, 2022.

(51) Int. Cl. C12Q 1/6825; C12Q 1/6837; C12Q 1/6876

(52) U.S. Cl. CPC C12Q 1/6825; C12Q 1/6837; C12Q 1/6876

(57) ABSTRACT

Methods of formation and detection of arrays of single analytes on enhanced substrates are described. The arrays may comprise pluralities of single analytes containing heterogeneity with respect to one or more properties. Enhanced substrates may be utilized to amplify the relative detection of optical signals form single analytes or moieties attached to single analytes with respect to sources of background, baseline, or erroneous optical signals.

### CROSS REFERENCE TO RELATED APPLICATIONS

This application is a 371 U.S. National Phase Application of International Patent Cooperation Treaty Application No. PCT/US2023/068798, entitled “Method for Detecting Analytes at Sites of Optically Non-Resolvable Distances,” filed Jun. 21, 2023, which claims priority to U.S. Provisional Patent Application Ser. No. 63/354,169, entitled, “Optical Signal Enhancement on Patterned Analyte Arrays,” filed Jun. 21, 2022, each of which is hereby incorporated by reference in its entirety for all purposes.

### BACKGROUND

Single-analyte processes and assays are often performed in an array-based format. Arrangement of single analytes on an array permits a degree of control over the positions and spacings of the single analytes. Arrays can also provide a measure of control for single-analyte processes or assays by preventing deposition or accumulation of moieties at improper locations on the array.

When performing single-analyte processes or assays, it is often necessary to acquire information on single analytes on an array at single-analyte resolution. Single-analyte resolution may be achieved by detecting a single analyte via a signal that is sufficiently distinct from a background or baseline signal. Alternatively or additionally, single-analyte resolution may be achieved by temporally and/or spatially identifying and/or differentiating each of two or more adjacent single analytes.

### SUMMARY

In an aspect, provided herein is a method, comprising: a) providing an array, wherein the array comprises: i) a plurality of sites, wherein each site of the plurality of sites is configured to bind a single analyte; ii) one or more interstitial regions, wherein each site of the plurality of sites is separated by the one or more interstitial regions from each other site of the plurality of sites; and iii) a layer disposed on a solid support, wherein the layer comprises a first thickness at the site, and wherein the layer comprises a second thickness at the interstitial region; b) coupling a first single analyte to a first site of the plurality of sites and a second single analyte to a second site of the plurality of sites, wherein the first single analyte differs from the second single analyte; and c) detecting presence of a first signal from the first site, presence of a second signal from the second site, and absence of signal from an interstitial region of the one or more interstitial regions; wherein an index of refraction of the solid support is greater than an index of refraction of the layer.

In some embodiments, providing the plurality of sites comprises forming each site of the plurality of sites on the solid support by a lithographic method. In some embodiments, the method further comprises disposing the layer on the solid support. In some embodiments, disposing the layer on the solid support occurs before forming each site of the plurality of sites. In some embodiments, disposing the layer on the solid support occurs after forming each site of the plurality of sites.

In some embodiments, the first single analyte or the second single analyte is coupled to an anchoring moiety. In some embodiments, the anchoring moiety is configured to couple the first single analyte or the second single analyte to a site of the plurality of sites. In some embodiments, the anchoring moiety is further configured to inhibit binding of the single analyte to the site of the plurality of sites. In some embodiments, the anchoring moiety comprises a nanoparticle, a nucleic acid, or a polypeptide. In some embodiments, the nucleic acid comprises a structured nucleic acid particle. In some embodiments, the structured nucleic acid particle comprises a nucleic acid origami or a nucleic acid nanoball.

In some embodiments, the first single analyte or the second single analyte comprises a biomolecule selected from the group consisting of polypeptide, polynucleotide, polysaccharide, lipid, metabolite, pharmaceutical compound, or a combination thereof. In some embodiments, the first single analyte differs from the second single analyte with respect to a difference in type of single analyte, species of single analyte, chemical property, physical property, or a combination thereof. In some embodiments, the difference in physical property comprises a difference in single analyte hydrodynamic radius, single analyte length, single analyte residue sequence, single analyte mass, single analyte net electrical charge, single analyte charge density, or a combination thereof.

In some embodiments, detecting the presence of the first signal or the presence of the second signal comprises detecting the first signal or the second signal with a signal-to-noise ratio of at least 2. In some embodiments, the absence of signal comprises a signal-to-noise ratio of less than 2. In some embodiments, the detecting comprises optically detecting the presence of the first signal or the presence of the second signal. In some embodiments, optical detection is performed on an optical detection system. In some embodiments, the optical detection system utilizes optical microscopy, surface plasmon resonance, infrared spectroscopy, ultraviolet spectroscopy, or a combination thereof.

In some embodiments, the detecting comprises: i) coupling a first detectable label to the first single analyte and a second detectable label to the second single analyte; and ii) detecting the presence of the first signal from the first detectable label at the first site and the presence of the second signal from the second detectable label at the second site. In some embodiments, the first detectable label or the second detectable label comprises an affinity agent. In some embodiments, the first detectable label or the second detectable label comprises a fluorophore or a luminophore. In some embodiments, the method further comprises removing the first detectable label from the first single analyte or the second detectable label from the second single analyte. In some embodiments, the removing step occurs before the detecting step. In some embodiments, the removing step occurs after the detecting step. In some embodiments, the removing step comprises a degradation reaction.

In some embodiments, the method further comprises: i) coupling a third detectable label to the interstitial region of the one or more interstitial regions; and ii) detecting the absence of a third signal from the third detectable label at the interstitial region. In some embodiments, distance of the first detectable label to the layer differs from distance of the second detectable label to the layer. In some embodiments, distance of the first detectable label or the second detectable label to the solid support differs from distance of the third detectable label to the solid support. In some embodiments, the index of refraction of the solid support is larger than the index of refraction of the layer by at least 1. In some embodiments, the second thickness is greater than 0.1 nanometers. In some embodiments, the layer comprises a metal, a metal oxide, a dielectric material, or a combination thereof.

In some embodiments, the method further comprises, before providing the array, determining the first thickness and the second thickness of the layer. In some embodiments, the first thickness or the second thickness is determined empirically. In some embodiments, the first thickness or the second thickness is determined computationally or theoretically. In some embodiments, the layer further comprises a passivating moiety. In some embodiments, the passivating moiety is configured to inhibit binding of a moiety to the layer. In some embodiments, the passivating moiety is coupled to the array at a site of the plurality of sites. In some embodiments, the passivating moiety is coupled to the array at the interstitial region of the one or more interstitial regions.

In another aspect, provided herein is a composition, comprising: a) a solid support; b) a layer disposed upon the solid support, wherein the layer comprises raised features of a first average thickness and indented features of a second average thickness; c) a plurality of anchoring moieties coupled to the layer; and d) a plurality of single analytes, wherein each single analyte is coupled to one and only one anchoring moiety of the plurality of anchoring moieties; wherein an index of refraction of the solid support is greater than an index of refraction of the layer.

In some embodiments, each anchoring moiety of the plurality of anchoring moieties is coupled to a single raised feature. In some embodiments, each anchoring moiety of the plurality of anchoring moieties is coupled to a single indented feature. In some embodiments, an anchoring moiety of the plurality of anchoring moieties is covalently coupled to the layer. In some embodiments, an anchoring moiety of the plurality of anchoring moieties is non-covalently coupled to the layer. In some embodiments, a single analyte of the plurality of single analytes is covalently coupled to an anchoring moiety of the plurality of anchoring moieties. In some embodiments, a single analyte of the plurality of single analytes is non-covalently coupled to an anchoring moiety of the plurality of anchoring moieties.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIGS. 1A, 1B, 1C, 1D, 1E, 1F and 1G depict various configurations of arrays comprising layers disposed upon solid supports, in accordance with some embodiments.

FIG. 2 shows an array comprising a heterogeneous plurality of analytes, in which each analyte has a differing size, in accordance with some embodiments.

FIGS. 3A and 3B illustrate array compositions that uniformize a distance of analytes from a surface of a solid support for analytes of differing size, in accordance with some embodiments.

FIGS. 4A, 4B, and 4C demonstrate a method of forming an array of analytes and contacting the array with a detectable agent that is configured to bind to some analytes of the array, in accordance with some embodiments.

FIGS. 5A, 5B, and 5C illustrate a hexagonal pattern of array sites with increasing site density, in accordance with some embodiments.

FIGS. 6A, 6B, 6C, and 6D depict a method of detecting detectable probe binding at optically non-resolvable sites utilizing immobilized avidity components, in accordance with some embodiments. FIG. 6E depicts an alternative array configuration containing a layered or deposited material for the method of FIGS. 6A-6D, in accordance with some embodiments.

FIGS. 7A, 7B, and 7C display multi-height array configurations for obtaining increased array site density, in accordance with some embodiments.

FIGS. 8A and 8B show additional multi-height array configurations, in accordance with some embodiments.

FIGS. 9A and 9B illustrate additional multi-height array configurations, in accordance with some embodiments.

FIG. 10 depicts an array system configuration utilizing a single sensor with multiple detection channels for detecting a multi-height array, in accordance with some embodiments.

FIG. 11 illustrates an optical system for detecting multiple wavelengths of light, in accordance with some embodiments.

FIG. 12 shows additional components of an array-based assay system, in accordance with some embodiments.

FIG. 13 illustrates a system for detection of a multi-height array utilizing multiple focal planes, in accordance with some embodiments.

FIG. 14 depicts processing of signals from multiple focal planes to spatially resolve array sites of a high-density analyte array, in accordance with some embodiments.

### DETAILED DESCRIPTION

Single-analyte systems may describe any system in which a plurality of moieties (e.g., single molecules, single nanoparticles, single microparticles, single colloids, single cells, etc.) are provided in a format such that each moiety of the plurality of moieties is individually addressable. For example, a polypeptide assay may be characterized as a single-molecule assay if each polypeptide of a plurality of polypeptides is disposed on an array such that: 1) each polypeptide is located at a fixed position on the array, and 2) each fixed position on the array contains no more than one polypeptide. Single-analyte processes and assays can be configured to simultaneously provide single-analyte systems that can efficiently organize pluralities of single analytes in a single-analyte format and provide a method of detection that can detect each single analyte at single-analyte resolution. Single-analyte resolution, in reference to a detection method or device of a single-analyte system, may have one or more properties of: 1) being configured to detect a single-analyte via a detectable signal that exceeds a background or baseline signal of the single-analyte system, and 2) being configured to spatially and/or temporally differentiate a single analyte from other analytes in the system (e.g., differentiating a first single analyte from a second single analyte that is adjacent to the first single analyte).

As spatial-scales and/or time-scales are reduced, optical detection of single analytes at single-analyte resolution becomes increasingly challenging. As array feature size is decreased from the microscale into the nanoscale, it becomes more difficult to resolve optical signals from an array to permit detection of single analytes above a background or baseline signal (e.g., due to autofluorescence) and to permit differentiation of one single analyte from another. Light collection can be increased by increasing collection time, but this often comes at the expense of detrimental physical processes, such as photobleaching or photodamage. Moreover, deposition of misplaced moieties on a single-analyte array (e.g., due to non-specific binding) or improper deposition of single analytes at improper locations on the single-analyte array can lead to false or misplaced signals that reduce or eliminate single-analyte resolution when detecting a single-analyte array.

Constructive and destructive interference provide a mechanism for enhancing wanted optical signals and minimizing misplaced optical signals on solid supports. Such methods can function by forming an enhanced solid support, in which a material possessing a smaller index of refraction is layered on a solid support containing a larger index of refraction. The differing refractive behaviors of the layered material and the solid support relative to emitted signals from detectable analytes give rise to regions of constructive or destructive optical interference. Consequently, if a signal source (e.g., a fluorophore, a luminophore, etc.) is located at a distance relative to the enhanced solid support that experiences constructive interference, a signal from the signal source will be enhanced relative to the same signal emitted relative to a non-enhanced solid support. Likewise, if a signal source is located at a distance relative to the enhanced solid support that experiences destructive interference, a signal from the signal source will be minimized or cancelled relative to the same signal emitted relative to a non-enhanced solid support. Solid supports for enhanced optical detection have been proposed in, for example, U.S. Pat. No. 7,988,918B2 and Lambacher, et al., Appl. Phys. A, vol. 63 (2000), each of which is herein incorporated by reference in its entirety.

Determination of an optimal thickness of a layered material on a solid support for signal enhancement becomes more difficult when a single-analyte array is to be formed from a heterogeneous plurality of single analytes. For example, a protein assay that is performed on a proteome-scale or subproteome-scale sample may be reasonably expected to contain hundreds to thousands of unique species of proteins, with those unique species of proteins distributed over a scale of amino acid sequence length spanning at least an order of magnitude. Whether in a condensed form or a partially- or fully-denatured form, the proteins of such a sample may contain a large variability in average or total distance relative to a solid support of an array to which the proteins are bound. Accordingly, some proteins may produce signals that are amplified while other proteins may produce signals that are deamplified based upon their relative distance to the solid support.

Set forth herein are systems and methods for increasing the relative difference between signals produced by analytes and signals produced by misplaced signal sources (e.g., non-specific binding, autofluorescence, etc.). The described methods and system utilize patterned, structured substrates to control the positioning of analytes on the substrates and control the relative amplification of signals originating from different locations on the substrates. The described substrates contain solid support with patterned layers of materials, in which the solid support and the layered materials have differing indexes of refraction. Arrays of analytes, including arrays of heterogeneous collections of analytes, can be prepared and detected on the provided substrates. Also provided herein are methods of assaying collections of analytes via optical detection systems that incorporate the signal-enhancing substrates, as set forth herein.

### Definitions

As used herein, the terms “address” and “site” synonymously refer to a location in an array where a particular analyte (e.g., protein, peptide, or unique identifier label) is present. An address can contain a single analyte, or it can contain a population of several analytes of the same species (i.e. an ensemble of the analytes). Alternatively, an address can include a population of different analytes. Addresses are typically discrete. The discrete addresses can be contiguous, or they can be separated by interstitial spaces. An array useful herein can have, for example, addresses that are separated by less than 100 microns, 10 microns, 1 micron, 100 nm, 10 nm or less. Alternatively or additionally, an array can have addresses that are separated by at least 10 nm, 100 nm, 1 micron, 10 microns, or 100 microns. The addresses can each have an area of less than 1 square millimeter, 500 square microns, 100 square microns, 10 square microns, 1 square micron, 100 square nm or less. An array can include at least about 1×10⁴, 1×10⁵, 1×10⁶, 1×10⁷, 1×10⁸, 1×10⁹, 1×10¹⁰, 1×10¹¹, 1×10¹², or more addresses.

As used herein, the term “affinity agent” refers to a molecule or other substance that is capable of specifically or reproducibly binding to an analyte (e.g., protein). An affinity agent can be larger than, smaller than or the same size as the analyte. An affinity agent may form a reversible or irreversible bond with an analyte. An affinity agent may bind with an analyte in a covalent or non-covalent manner. Affinity agents may include reactive affinity agents, catalytic affinity agents (e.g., kinases, proteases, etc.) or non-reactive affinity agents (e.g., antibodies or fragments thereof). An affinity agent can be non-reactive and non-catalytic, thereby not permanently altering the chemical structure of an analyte to which it binds. Affinity agents that can be particularly useful for binding to proteins include, but are not limited to, antibodies or functional fragments thereof (e.g., Fab′ fragments, F(ab′)₂ fragments, single-chain variable fragments (scFv), di-scFv, tri-scFv, or microantibodies), affibodies, affilins, affimers, affitins, alphabodies, anticalins, avimers, DARPins, monobodies, nanoCLAMPs, nucleic acid aptamers, protein aptamers, lectins or functional fragments thereof.

As used herein, the term “anchoring moiety” refers to a moiety, molecule, or particle that serves as an intermediary attaching a protein or peptide to a surface (e.g., a solid support or a microbead). An anchoring moiety may be covalently or non-covalently attached to a surface and/or a polypeptide. An anchoring moiety may be a biomolecule, polymer, particle, nanoparticle, or any other entity that is capable of attaching to a surface or polypeptide. In some cases, an anchoring moiety may be a structured nucleic acid particle.

As used herein, the term “array” refers to a population of analytes (e.g., proteins) or a population of sites that are configured to bind analytes that are associated with unique identifiers such that the analytes can be distinguished from each other. A unique identifier can be, for example, a solid support (e.g., particle or bead), address on a solid support, tag, label (e.g., luminophore), or barcode (e.g., nucleic acid barcode) that is associated with an analyte and that is distinct from other identifiers in the array. Analytes can be associated with unique identifiers by attachment, for example, via covalent bonds or non-covalent bonds (e.g., ionic bond, hydrogen bond, van der Waals forces, electrostatics etc.). An array can include different analytes that are each attached to different unique identifiers. An array can include different unique identifiers that are attached to the same or similar analytes. An array can include separate solid supports or separate addresses that each bear a different analyte, wherein the different analytes can be identified according to the locations of the solid supports or addresses.

As used herein, the terms “attached” and “coupled” refer synonymously to the state of two things being joined, fastened, adhered, connected, or bound to each other. Attachment can be covalent or non-covalent. For example, a particle can be attached to a protein by a covalent or non-covalent bond. A covalent bond is characterized by the sharing of pairs of electrons between atoms. A non-covalent bond is a chemical bond that does not involve the sharing of pairs of electrons and can include, for example, hydrogen bonds, ionic bonds, van der Waals forces, hydrophilic interactions, adhesion, adsorption, and hydrophobic interactions.

As used herein, the term “avidity component” refers to a moiety of a first binding partner that is configured to interact with a moiety of a second binding partner to increase the rate of association between the first and second binding partners and/or to decrease the rate of dissociation the first and second binding partners. The first binding partner can further include a primary epitope moiety that interacts with a primary paratope moiety of the second binding partner, or vice versa. An avidity component can include a polymer, nucleic acid strand, nucleic acid duplex, nucleotide sequence, protein, affinity reagent, secondary epitope, secondary paratope, receptor, ligand or the like. A first avidity component can interact with a second avidity component via reversible binding, for example, via non-covalent binding or reversible covalent binding. As used herein, the term “binding specificity” refers to the tendency of a detectable probe, or an affinity reagent or avidity component thereof, to preferentially interact with an affinity target or avidity target, respectively. A detectable probe, or an affinity reagent or avidity component thereof, may have an observed, known, or predicted binding specificity for any possible binding partner, affinity target, or target moiety. Binding specificity may refer to selectivity for a single detectable probe, affinity target, or avidity target on an array over at least one other possible binding partner on the array. Moreover, binding specificity may refer to selectivity for a subset of affinity targets or avidity targets on an array over at least one other binding partner on the array.

As used herein, the term “binding affinity” refers to the strength or extent of binding between a detectable probe, or an affinity reagent or avidity component thereof, and a binding partner. In some cases, the binding affinity of a detectable probe, or an affinity reagent or avidity component thereof, for a binding partner may be vanishingly small or effectively zero. A binding affinity of a detectable probe, or an affinity reagent—or avidity component thereof, for a binding partner may be qualified as being a “high affinity,” “medium affinity,” or “low affinity.” A binding affinity—of a detectable probe, or an affinity reagent or avidity component thereof, for a binding partner may be quantified as being “high affinity” if the interaction has a dissociation constant of less than about 100 nM, “medium affinity” if the interaction has a dissociation constant between about 100 nM and 1 mM, and “low affinity” if the interaction has a dissociation constant of greater than about 1 mM. Binding affinity—can be described in terms known in the art of biochemistry such as equilibrium dissociation constant (K_{D}), equilibrium association constant (K_{A}), association rate constant (kₒₙ), dissociation rate constant (k_{off}) and the like. See, for example, Segel, Enzyme Kinetics John Wiley and Sons, New York (1975), which is incorporated herein by reference in its entirety.

As used herein, the term “bioorthogonal reaction” refers to a chemical reaction that can occur within a biological system (in vitro and/or in vivo) without interfering with some or all native biological processes, functions, or activities of the biological system. A bioorthogonal reaction may be further characterized as being inert to components of a biological system other than those targeted by the bioorthogonal reaction. A bioorthogonal reaction may include a click reaction. A bioorthogonal reaction may utilize an enzymatic reaction, such as attachment between a first molecule and a second molecule by an enzyme such as a sortase, a ligase, or a subtiligase. A bioorthogonal reaction may utilize an irreversible peptide capture system, such as SpyCatcher/SpyTag, SnoopCatcher/SnoopTag, or SdyCatcher/SdyTag.

As used herein, the term “click-type reaction” refers to single-step, thermodynamically-favorable conjugation reaction utilizing biocompatible reagents. A click reaction may be configured to not utilize toxic or biologically incompatible reagents (e.g., acids, bases, heavy metals) or to not generate toxic or biologically incompatible byproducts. A click reaction may utilize an aqueous solvent or buffer (e.g., phosphate buffer solution, Tris buffer, saline buffer, MOPS, etc.). A click reaction may be thermodynamically favorable if it has a negative Gibbs free energy of reaction, for example a Gibbs free energy of reaction of less than about −5 kiloJoules/mole (KJ/mol), −10 KJ/mol, −25 KJ/mol, −100 KJ/mol, −250 KJ/mol, −500 KJ/mol, or less. Exemplary click reactions may include metal-catalyzed azide-alkyne cycloaddition, strain-promoted azide-alkyne cycloaddition, strain-promoted azide-nitrone cycloaddition, strained alkene reactions, thiol-ene reaction, Diels-Alder reaction, inverse electron demand Diels-Alder reaction (IEDDA), [3+2] cycloaddition, [4+1] cycloaddition, nucleophilic substitution, dihydroxylation, thiol-yne reaction, photoclick, nitrone dipole cycloaddition, norbornene cycloaddition, oxanobornadiene cycloaddition, tetrazine ligation, and tetrazole photoclick reactions. Exemplary reactive moieties utilized to perform click reactions may include alkenes, alkynes, azides, epoxides, amines, thiols, nitrones, isonitriles, isocyanides, aziridines, activated esters, and tetrazines. Other well-known click conjugation reactions may be used having complementary bioorthogonal reaction species, for example, where a first click component comprises a hydrazine moiety and a second click component comprises an aldehyde or ketone group, and where the product of such a reaction comprises a hydrazone functional group or equivalent. Exemplary bioorthogonal and click reactions are set forth in US Pat. App. Pub. No. 2021/0101930 A1, which is incorporated herein by reference.

The term “comprising” is intended herein to be open-ended, including not only the recited elements, but further encompassing any additional elements.

As used herein, the term “detectable probe” refers to an affinity agent that is coupled to a detectable label. Optionally, a detectable probe may further comprise an avidity component. A detectable probe may further incorporate a linking moiety, such as a polymer linker or a nanoparticle, that couples together one or more components (e.g., affinity agent, detectable label, and/or avidity component) of the detectable probe.

As used herein, the term “each,” when used in reference to a collection of items, is intended to identify an individual item in the collection but does not necessarily refer to every item in the collection. Exceptions can occur if explicit disclosure or context clearly dictates otherwise.

As used herein, the term “enhanced substrate” refers to a solid support comprising a layered or deposited material that is disposed on a surface of the solid support. A layered or deposited material may include a metal, metal oxide, semiconductor, polymer, glass, dielectric material, or a combination thereof. The solid support and/or the layered or deposited material may be structured (e.g., lithographically formed). The solid support of an enhanced substrate may contain a substantially planar surface or a non-planar surface upon which the layered or deposited material is disposed. A layered or deposited material disposed on a solid support may comprise a substantially planar surface, or a plurality of surfaces that are substantially coplanar. An external surface of an enhanced substrate may comprise one or more raised features, and/or one or more indented features. A surface of a solid support of an enhanced substrate may comprise areas of exposed solid support and areas of solid support that are covered in a layered or deposited material. A surface of a solid support of an enhanced substrate may comprise no areas of exposed solid support. An enhanced substrate may be characterized as producing a differential interaction between photons of light with the solid support and photons of light with the layered or deposited material. Accordingly, an enhanced substrate may produce constructive or destructive interference of optical signals as a function of distance between a surface of the solid support or surface of the layered or deposited material and an optical signal source. An enhanced substrate may comprise an array.

As used herein, the term “epitope” refers to an affinity target within a protein, polypeptide, or other analyte. Epitopes may include amino acid sequences that are sequentially adjacent in the primary structure of a protein. Epitopes may include amino acids that are structurally adjacent in the secondary, tertiary, or quaternary structure of a protein despite being non-adjacent in the primary sequence of the protein. An epitope can be, or can include, a moiety of protein that arises due to a post-translational modification, such as a phosphate, phosphotyrosine, phosphoserine, phosphothreonine, or phosphohistidine. An epitope can optionally be recognized by or bound to an antibody. However, an epitope need not necessarily be recognized by any antibody, for example, instead being recognized by an aptamer, mini-protein, or other affinity reagent. An epitope can optionally bind an antibody to elicit an immune response. However, an epitope need not necessarily participate in, nor be capable of, eliciting an immune response.

As used herein, the term “paratope” refers to a molecule or moiety which recognizes or binds specifically to an epitope. A paratope may include an antigen binding site of an antibody. A paratope may include at least 1, 2, 3, or more complementarity-determining regions of an antibody. A paratope need not necessarily be present in nor derived from an antibody, for example, instead being present in a nucleic acid aptamer, lectin, streptavidin, miniprotein or other affinity reagent. A paratope need not necessarily participate in, nor be capable of, eliciting an immune response.

As used herein, the terms “group” and “moiety” are intended to be synonymous when used in reference to the structure of a molecule. The terms refer to a component or part of the molecule. The terms do not necessarily denote the relative size of the component or part compared to the rest of the molecule, unless indicated otherwise.

As used herein, the terms “label” and “detectable label” synonymously refer to a molecule or moiety that provides a detectable characteristic. The detectable characteristic can be, for example, an optical signal such as absorbance of radiation, luminescence emission, luminescence lifetime, luminescence polarization, fluorescence emission, fluorescence lifetime, fluorescence polarization, or the like; Rayleigh and/or Mie scattering; binding affinity for a ligand or receptor; magnetic properties; electrical properties; charge; mass; radioactivity or the like. Exemplary labels include, without limitation, a fluorophore, luminophore, chromophore, nanoparticle (e.g., gold, silver, carbon nanotubes), heavy atoms, radioactive isotope, mass label, charge label, spin label, receptor, ligand, or the like. A label may produce a signal that is detectable in real-time (e.g., fluorescence, luminescence, radioactivity). A label may produce a signal that is detected off-line (e.g., a nucleic acid barcode) or in a time-resolved manner (e.g., time-resolved fluorescence). A label may produce a signal with a characteristic frequency, intensity, polarity, duration, wavelength, sequence, or fingerprint.

As used herein, the terms “linking group,” or “linking moiety” refer to a moiety, molecule or molecular chain that is configured to attach a first molecule to a second molecule. A linker, linking group, or linking moiety may be configured to provide a chemical or mechanical property to a region separating a first molecule from a second molecule, such as hydrophobicity, hydrophilicity, electrical charge, polarity, rigidity, or flexibility. A linker, linking group, or linking moiety may comprise two or more functional groups that facilitate the coupling of the linker, linking group, or linking moiety to the first and second molecule. A linker, linking group, or linking moiety may include polyfunctional linkers such as homobifunctional linkers, heterobifunctional linkers, homopolyfunctional linkers, and heteropolyfunctional linkers. The molecular chain may be characterized by a minimum size such as, for example, at least about 100 Daltons (Da), 200 Da, 300 Da, 400 Da, 500 Da, 600 Da, 700 Da, 800 Da, 900 Da, 1 kiloDalton (kDa), 2 kDa, 3 kDa, 4 kDa, 5 kDa, 10 kDa, 15 kDa, 20 kDa or more than 20 kDa. Alternatively or additionally, a molecular chain may be characterized by a maximum size such as, for example, no more than about 20 kDa, 15 kDa, 10 kDa, 5 kDa, 4 kDa, 3 kDa, 2 kDa, 1 kDa, 900 Da, 800 Da, 700 Da, 600 Da, 500 Da, 400 Da, 300 Da, 200 Da, 100 Da, or less than 100 Da. Exemplary molecular chains may comprise polyethylene glycol (PEG), polyethylene oxide (PEO), alkane chains, fluorinated alkane chains, dextrans, and polynucleotides.

As used herein, the term “misplaced,” when used in reference to an array, refers to a moiety, molecule, label, signal source, or particle being located or co-located at an unintended address or site of the array. A misplaced moiety, molecule, label, signal source, or particle may become located at an improper address of an array due to a non-specific binding interaction (i.e., unexpected, unwanted, or unlikely binding of the moiety, molecule, label, signal source, or particle to a site, single analyte, or moiety coupled to the array). A single analyte or a moiety attached thereto (e.g., an affinity agent) may be misplaced if co-located at a site with a second single analyte or moiety attached thereto. For example, at a site with two coupled single analytes, one or both of the first and second single analyte may be considered misplaced if the site is only intended to bind one single analyte.

As used herein, the terms “nucleic acid nanostructure” or “nucleic acid nanoparticle,” refer synonymously to a single- or multi-chain polynucleotide molecule comprising a compacted three-dimensional structure. The compacted three-dimensional structure can optionally have a characteristic tertiary structure. An exemplary nucleic acid nanostructure is a structured nucleic acid particle (SNAP). A SNAP can be configured to have an increased number of interactions between regions of a polynucleotide strand, less distance between the regions, increased number of bends in the strand, and/or more acute bends in the strand, as compared to the same nucleic acid molecule in a random coil or other non-structured state. Alternatively or additionally, the compacted three-dimensional structure of a nucleic acid nanostructure can optionally have a characteristic quaternary structure. For example, a nucleic acid nanostructure can be configured to have an increased number of interactions between polynucleotide strands or less distance between the strands, as compared to the same nucleic acid molecule in a random coil or other non-structured state. In some configurations, the tertiary structure (i.e. the helical twist or direction of the polynucleotide strand) of a nucleic acid nanostructure can be configured to be denser than the same nucleic acid molecule in a random coil or other non-structured state. Nucleic acid nanostructures may include deoxyribonucleic acid (DNA), ribonucleic acid (RNA), peptide nucleic acid (PNA), other nucleic acid analogs, and combinations thereof. Nucleic acid nanostructures may have naturally-arising or engineered secondary, tertiary, or quaternary structures. A structured nucleic acid particle can contain at least one of: i) a moiety that is configured to couple an analyte to the nucleic acid nanostructure, ii) a moiety that is configured to couple the nucleic acid nanostructure to another object such as another SNAP, a solid support or a surface thereof, iii) a moiety that is configured to provide a chemical or physical property or characteristic to a nucleic acid nanostructure, or iv) a combination thereof. Exemplary SNAPs may include nucleic acid nanoballs (e.g., DNA nanoballs), nucleic acid nanotubes (e.g., DNA nanotubes), and nucleic acid origami (e.g., DNA origami). A SNAP may be functionalized to include one or more reactive handles or other moieties. A SNAP may comprise one or more incorporated residues that contain reactive handles or other moieties (e.g., modified nucleotides).

As used herein, the term “nucleic acid nanoball” refers to a globular or spherical nucleic acid structure. A nucleic acid nanoball may comprise a concatemer of oligonucleotides that arranges in a globular structure. A nucleic acid nanoball may comprise one or more oligonucleotides, including oligonucleotides comprising self-complementary nucleic acid sequences. A nucleic acid nanoball may comprise a palindromic nucleic acid sequence. A nucleic acid nanoball may include DNA, RNA, PNA, LNAs, other nucleic acid analog, modified or non-natural nucleic acids, or combinations thereof.

As used herein, the term “nucleic acid origami” refers to a nucleic acid construct having an engineered tertiary or quaternary structure. A nucleic acid origami may include DNA, RNA, PNA, modified or non-natural nucleic acids, or combinations thereof. A nucleic acid origami may include a plurality of oligonucleotides that hybridize via sequence complementarity to produce the engineered structuring of the origami. A nucleic acid origami may include sections of single-stranded or double-stranded nucleic acid, or combinations thereof. Exemplary nucleic acid origami structures may include nanotubes, nanowires, cages, tiles, nanospheres, blocks, and combinations thereof. A nucleic acid origami can optionally include a relatively long scaffold nucleic acid to which multiple smaller nucleic acids hybridize, thereby creating folds and bends in the scaffold that produce an engineered structure. The scaffold nucleic acid can be circular or linear. The scaffold nucleic acid can be single stranded but for hybridization to the smaller nucleic acids. A smaller nucleic acid (sometimes referred to as a “staple”) can hybridize to two regions of the scaffold, wherein the two regions of the scaffold are separated by an intervening region that does not hybridize to the smaller nucleic acid.

As used herein, the term “optically resolvable distance,” when used in reference to two array sites, refers to a spatial separation between two array sites that is at least minimally sufficient to distinguish separate optical signals from both array sites with an optical detection device.

As used herein, the terms “protein” and “polypeptide” refer synonymously to a molecule comprising two or more amino acids joined by a peptide bond. A protein may also be referred to as a polypeptide, oligopeptide, or peptide. A protein can be a naturally-occurring molecule, or synthetic molecule. A protein may include one or more non-natural amino acids, modified amino acids, or non-amino acid linkers. A protein may contain D-amino acid enantiomers, L-amino acid enantiomers or both. Amino acids of a protein may be modified naturally or synthetically, such as by post-translational modifications. In some circumstances, different proteins may be distinguished from each other based on different genes from which they are expressed in an organism, different primary sequence length or different primary sequence composition. Proteins expressed from the same gene may nonetheless be different proteoforms, for example, being distinguished based on non-identical length, non-identical amino acid sequence or non-identical post-translational modifications. Different proteins can be distinguished based on one or both of gene of origin and proteoform state.

As used herein, the term “single,” when used in reference to an object such as an analyte, means that the object is individually manipulated or distinguished from other objects. A single analyte can be a single molecule (e.g., single protein), a single complex of two or more molecules (e.g., a multimeric protein having two or more separable subunits, a single protein attached to a structured nucleic acid particle, or a single protein attached to an affinity reagent), a single particle, or the like. Reference herein to a “single analyte” in the context of a composition, system or method herein does not necessarily exclude application of the composition, system or method to multiple single analytes that are manipulated or distinguished individually, unless indicated contextually or explicitly to the contrary.

As used herein, the term “single-analyte resolution” refers to the detection of, or ability to detect, an analyte on an individual basis, for example, as distinguished from its nearest neighbor in an array.

As used herein, the term “solid support” refers to a substrate that is insoluble in aqueous liquid. Optionally, the substrate can be rigid. The substrate can be non-porous or porous. The substrate can optionally be capable of taking up a liquid (e.g., due to porosity) but will typically, but not necessarily, be sufficiently rigid that the substrate does not swell substantially when taking up the liquid and does not contract substantially when the liquid is removed by drying. A nonporous solid support is generally impermeable to liquids or gases. Exemplary solid supports include, but are not limited to, glass and modified or functionalized glass, plastics (including acrylics, polystyrene and copolymers of styrene and other materials, polypropylene, polyethylene, polybutylene, polyurethanes, Teflon™, cyclic olefins, polyimides etc.), nylon, ceramics, resins, Zeonor™, silica or silica-based materials including silicon and modified silicon, germanium, carbon, metals, inorganic glasses, optical fiber bundles, gels, and polymers. In particular configurations, a flow cell contains the solid support such that fluids introduced to the flow cell can interact with a surface of the solid support to which one or more components of a binding event (or other reaction) is attached.

As used herein, the term “structured nucleic acid particle” or “SNAP” refers to a single- or multi-chain polynucleotide molecule having a compacted three-dimensional structure. The compacted three-dimensional structure can optionally be characterized in terms of hydrodynamic radius or Stoke's radius of the SNAP relative to a random coil or other non-structured state for a nucleic acid having the same sequence length as the SNAP. The compacted three-dimensional structure can optionally be characterized with regard to tertiary structure. For example, a SNAP can be configured to have an increased number of internal binding interactions between regions of a polynucleotide strand, less distance between the regions, increased number of bends in the strand, and/or more acute bends in the strand, as compared to a nucleic acid molecule of similar length in a random coil or other non-structured state. Alternatively or additionally, the compacted three-dimensional structure can optionally be characterized with regard to tertiary or quaternary structure. For example, a SNAP can be configured to have an increased number of interactions between polynucleotide strands or less distance between the strands, as compared to a nucleic acid molecule of similar length in a random coil or other non-structured state. In some configurations, the secondary structure of a SNAP can be configured to be denser than a nucleic acid molecule of similar length in a random coil or other non-structured state. A SNAP may contain DNA, RNA, PNA, modified or non-natural nucleic acids, or combinations thereof. A SNAP may include a plurality of oligonucleotides that hybridize to form the SNAP structure. The plurality of oligonucleotides in a SNAP may include oligonucleotides that are attached to other molecules (e.g., probes, analytes such as proteins, reactive moieties, or detectable labels) or are configured to be attached to other molecules (e.g., by functional groups). A SNAP may include engineered or rationally designed structures. Exemplary SNAPs include nucleic acid origami and nucleic acid nanoballs.

As used herein, the terms “type” and “species,” when used in reference to a subset of analytes, refers to a characteristic that is shared by the analytes in the subset and that distinguishes the analytes in the subset from analytes that are not in the subset. The characteristic can be any of a variety of characteristics known for the analytes. Any of a variety of analytes can be categorized by type, including for example, proteins. Exemplary characteristics that can be used to categorize proteins by type include, but are not limited to, amino acid composition, full length amino acid sequence, proteoform, presence or absence of an amino acid sequence motif, number of amino acids present (i.e. sequence length), molecular weight, presence or absence of a particular epitope, presence or absence of epitope(s) recognized by a particular affinity reagent, probability of binding a particular affinity reagent, presence or absence of a post-translational modification, enzymatic activity, affinity for binding a particular protein or protein motif, or the like.

### Enhanced Detection Systems and Methods

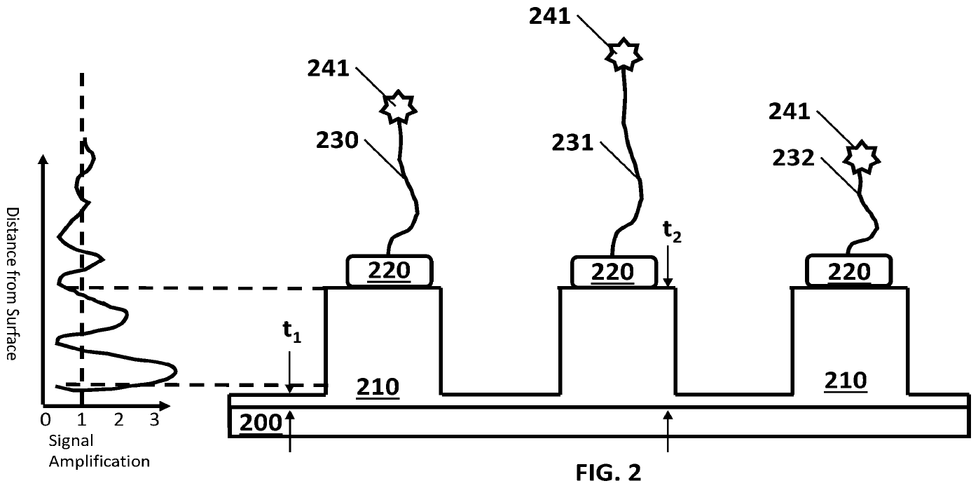

FIG. 2 depicts a configuration of an array of single analytes on an enhanced substrate, as set forth herein. The enhanced substrate comprises a substrate 200 with a coating comprising a layered or deposited material 210. The layered or deposited material 210 comprises a spatially varying thickness, with raised features having a maximum thickness, t₂, and indented features having a minimum thickness, t₁. Coupled to the upper surface of each raised feature of the layered or deposited material 210 is an anchoring moiety 220 (e.g., a nucleic acid, a polypeptide, a nanoparticle, etc.) that is configured to couple a single analyte to a site of the substrate. Each anchoring moiety 220 is coupled to a single analyte (230, 231, 232). Each single analyte comprises a different size, with single analyte 231 extending the longest distance from the surface of the enhanced substrate, and single analyte 232 extending the shortest distance from the surface of the enhanced substrate. Each single analyte comprises a terminal detectable label 241 (e.g., a fluorophore, a luminophore, a reflective particle, an absorptive particle, etc.) that is configured to produce a detectable optical signal. In the example shown, the single analytes (230, 231, 232) are attached to the surface via a first terminus and label 241 is located at the terminus of each analyte that is distal to the point of surface attachment. Due to the differing sizes of the single analytes, each detectable label 241 produces a detectable signal at a differing distance with respect to the enhanced substrate. To the right of the cross-sectional view of the enhanced substrate, a graph displays a qualitative result for an expected optical signal amplification as a function of thickness of the layered or deposited material 210. At very thin thicknesses (e.g., t₁ or thinner), an optical signal produced adjacent to the layered or deposited material 210 would be expected to be de-amplified (i.e., experiencing a signal factor of less than 1). As layer thickness increases, the signal amplification factor experiences nodes (i.e., maxima in signal amplification) and anti-nodes (i.e., minima in signal amplification). In the depicted configuration, the maximum thickness, t₂, is located at an anti-node for signal amplification, such that any detectable moieties bound directly to the surface may experience signal deamplification, thereby reducing signal from non-specific binding of unbound detectable labels 241. At larger distances, the degree of signal amplification and deamplification decrease. Accordingly, optical signals emerging from detectable labels 241 coupled to single analytes (230, 231, 232) may produce less variability in signal intensity due to differing label positions relative to the enhanced substrate. The array configuration depicted in FIG. 2 can be optimized to drive non-specific binding of misplaced optical signal sources to surfaces whose configuration facilitates signal deamplification, while analytes, or signal sources attached thereto, can be positioned relative to the enhanced substrate to facilitate signal amplification or minimize a likelihood and/or magnitude of signal de-amplification.

In an aspect, provided herein is a method, comprising: a) providing an array, wherein the array comprises: i) a plurality of sites, wherein each site of the plurality of sites is configured to bind a single analyte, ii) one or more interstitial regions, wherein each site of the plurality of sites is separated by the one or more interstitial regions from each other site of the plurality of sites, and iii) a layer disposed on a substrate, wherein the layer comprises a first thickness at the site, and wherein the layer comprises a second thickness at the interstitial region, b) coupling a first single analyte to a first site of the plurality of sites and a second single analyte to a second site of the plurality of sites, wherein the first single analyte differs from the second single analyte, and c) detecting a presence of a first signal from the first site, a presence of a second signal from the second site, and an absence of a third signal from an interstitial region of the one or more interstitial regions.

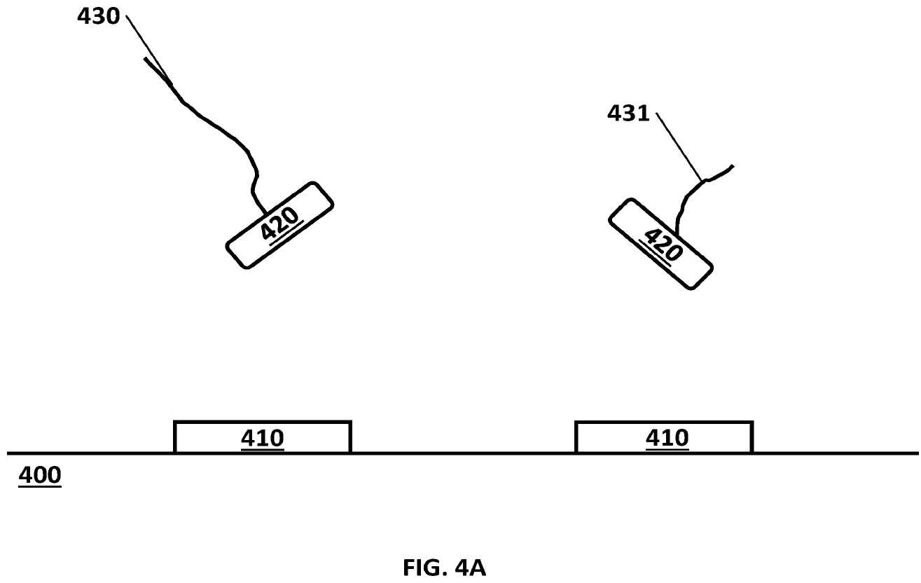

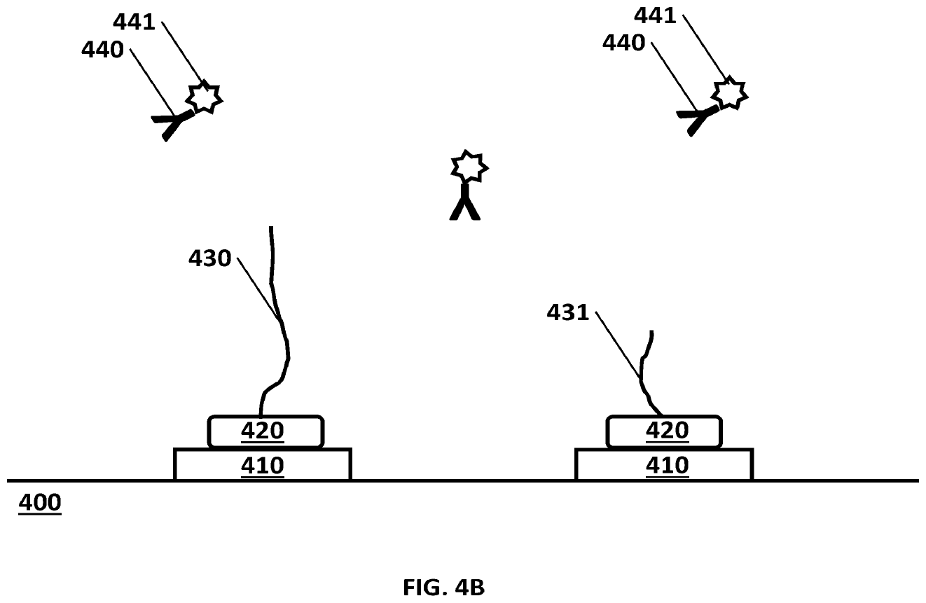

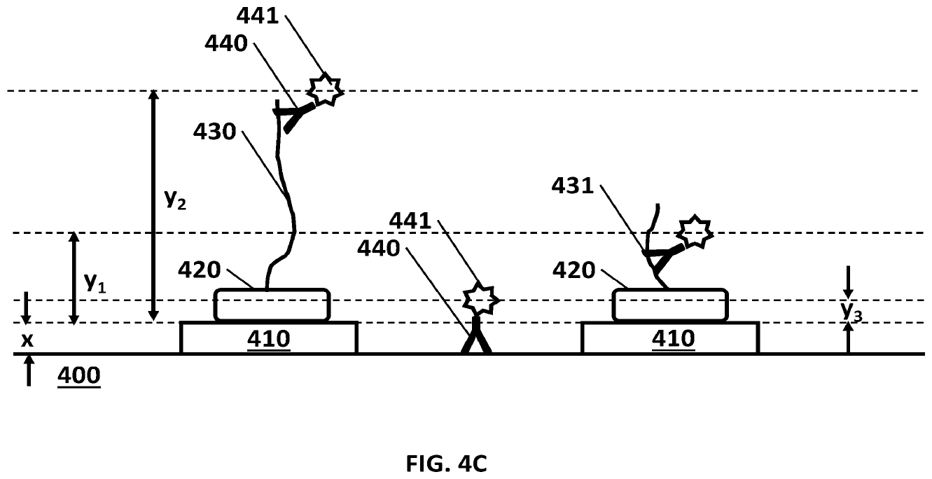

FIGS. 4A-4C depict a method of forming an array and performing a detection assay on an enhanced substrate. Referring to FIG. 4A, an enhanced substrate comprising a substrate 400 and raised features of thickness x comprising a layered or deposited material 410 may be contacted with a plurality of single analytes 430. Each single analyte 430 may be coupled to an anchoring moiety 420 that is configured to couple the single analyte 430 to the raised features of the enhanced substrate. Referring to FIG. 4B, after depositing a single analyte 430 at each raised feature by coupling an anchoring moiety 420 to the layered or deposited material 410, the array of single analytes 430 is contacted with a plurality of affinity agents 440. Each affinity agent 440 is configured to couple a moiety (e.g., a polypeptide epitope, a nucleotide sequence, etc.) that is known or suspected to be present in at least one single analyte 430 of the plurality of single analytes 430. Each affinity agent 440 comprises a detectable label 441 that is configured to produce a detectable optical signal. Referring to FIG. 4C, the array is depicted after one or more affinity agents 440 have coupled to addresses of the array. Affinity agents 440 have coupled to single analytes 430 and 431. Another affinity agent 440 has coupled to a surface of the solid support 400 due to an unwanted non-specific binding interaction. The detectable label 441 of the affinity agent 440 coupled to single analyte 430 is located a distance y₂ from the top surface of the raised feature. The detectable label 441 of the affinity agent 440 coupled to single analyte 431 is located a distance y₁ from the top surface of the raised feature. The detectable label 441 of the affinity agent 440 coupled to the substrate 400 is located a distance y₃ from the top surface of the raised feature. An optical signal emitted from each detectable label 441 may be amplified or de-amplified based upon its location (y₁, y₂, y₃, etc.) relative to the substrate 400 or layered or deposited material 410. Ideally, distance y₃ will be sufficient to de-amplify the misplaced optical signal from the non-specifically bound affinity agent 440. The steps depicted in FIGS. 4B-4C may be repeated with multiple affinity agents, with each affinity agent binding (or not binding) to single analytes (430, 431) at differing locations.

An array, as set forth herein, may comprise a plurality of sites that are configured to couple a single analyte. In some configurations, providing the plurality of sites comprises forming each site of the plurality of sites on the substrate by a lithographic method. Exemplary lithographic methods may include photolithography, Dip-Pen nanolithography, nanoimprint lithography, nanosphere lithography, nanoball lithography, nanopillar arrays, nanowire lithography, scanning probe lithography, thermochemical lithography, thermal scanning probe lithography, local oxidation nanolithography, molecular self-assembly, stencil lithography, deep ultraviolet patterning, or electron-beam lithography. An array may be formed by a lithographic method comprising one or more, two or more, or three or more steps of: 1) applying a protective layer (e.g., a photoresist, a masking material, etc.) to a surface of a substrate, 2) forming one or more structures on the surface of the substrate by a lithographic method, 3) forming a coating of a layered or deposited material on the surface of the substrate, 4) applying a protective layer (e.g., a photoresist, a masking material, etc.) to a surface of the layered or deposited material, and 5) forming one or more structures on the surface of the layered or deposited material by a lithographic method, and 6) removing at least a portion of the protective layer from the surface of the solid support or the layered or deposited material. The skilled person will readily recognize numerous variations of methods for forming an array, as set forth herein (e.g., lift-off methods).

A method, as set forth herein, may comprise disposing a layered or deposited material on a solid support. In some configurations, disposing a layer on a substrate can occur before forming each site of a plurality of sites. For example, a site or a plurality thereof may be formed by lithographically forming a uniform (e.g., spatially non-variant) coating of a layered or deposited material on a substrate. In other configurations, disposing a layer on a substrate can occur after forming each site of a plurality of sites. For example, a site or a plurality thereof may be formed by lithographically forming a substrate, then disposing a uniform or non-uniform coating of a layered or deposited material on the substrate.

A method of the present disclosure may comprise coupling a plurality of single analytes to an array, as set forth herein. In some configurations, each single analyte of a plurality of single analytes can be covalently attached to a site of a plurality of sites of an array. In other configurations, each single analyte of a plurality of single analytes can be non-covalently attached to a site of a plurality of sites of an array. In some configurations, a first single analyte and a second single analyte may be coupled to an array, in which the first single analyte or the second single analyte is coupled to the array via an anchoring moiety. An anchoring moiety may comprise a moiety that comprises one or more properties of: i) being configured to couple a single analyte to a site of a plurality of sites, and ii) being configured to inhibit binding of the single analyte to the site of the plurality of sites. For example, an anchoring moiety may comprise a functional group or surface that preferentially binds a site relative to binding of a single analyte to a site, or an anchoring moiety may comprise a structure that occludes or otherwise obstructs binding of a single analyte to the site. In some configurations, an anchoring moiety may be configured to occupy a site of an array such that a second anchoring moiety is occluded from binding to the occupied site. This can be achieved, for example, via steric exclusion due to size or shape of the anchoring moiety relative to size or shape of the array site, or via repulsion due to chemical characteristics such electrical charge (positive or negative), polarity, hydrophobicity, hydrophilicity or the like. Exemplary anchoring moieties are described in U.S. Pat. No. 11,203,612B2 and U.S. Pat. No. 11,505,796B2, each of which is herein incorporated by reference. In some configurations, an anchoring moiety can comprise a nanoparticle, a nucleic acid, a polypeptide, or a combination thereof. In particular configurations, an anchoring moiety can comprise a nucleic acid that comprises a structured nucleic acid particle (e.g., a nucleic acid origami, a nucleic acid nanoball). A structured nucleic acid particle can be configured to have a net-negative surface charge (e.g., due to the phosphate backbone of nucleic acids included in the particle), wherein a first structured nucleic acid particle is attracted to a positively charged site and a second structured nucleic acid is repelled from the occupied site due to repulsion between the negatively charged surfaces of the two particles.

An array, as set forth herein, may comprise a plurality of single analytes. A single analyte can comprise a biomolecule, a nanoparticle, a microparticle, a cell, a viral particle, a colloid, or a combination thereof. An array may comprise a plurality of biomolecules or biologically-relevant molecules (e.g., pharmaceuticals, toxins, etc.), in which a biomolecule or biologically-relevant molecule of the plurality of biomolecules or biologically-relevant molecules is selected from the group consisting of polypeptide, polynucleotide, polysaccharide, lipid, metabolite, pharmaceutical compound, toxin, or a combination thereof. An array may comprise a plurality of single analytes, in which the plurality of single analytes is homogeneous with respect to at least one property. For example, each single analyte of an array of single analytes may comprise a polypeptide. An array may comprise a plurality of single analytes, in which the plurality of single analytes is heterogeneous with respect to at least one property. For example, an array of polypeptide single analytes may comprise two or more unique species or proteoforms of polypeptides. An array may comprise a first single analyte and a second single analyte, in which the first single analyte differs from the second single analyte with respect to a difference in type of single analyte, species of single analyte, chemical property, physical property, or a combination thereof. A difference in physical property may comprise a difference in single analyte hydrodynamic radius, single analyte length, single analyte residue sequence, single analyte mass, single analyte net electrical charge, single analyte charge density, or a combination thereof.

An enhanced substrate, as set forth herein, may be utilized to increase a difference in optical signal magnitude between an optical signal from or pertaining to a single analyte and an optical signal from or pertaining to a misplaced moiety. For example, during detection of an array of single analytes, optical signals may be recorded from single analytes or moieties attached thereto and from non-specifically bound moieties (e.g., moieties that have undesirably bound to surfaces of the array). An enhanced substrate may de-amplify an optical signal produced by a misplaced moiety relative to an optical signal from a single analyte or a moiety attached thereto. An enhanced substrate may amplify an optical signal produced by a single analyte or a moiety attached thereto relative to a misplaced moiety. In some cases, an enhanced substrate may amplify an optical signal from a single analyte or a moiety attached thereto and an optical signal from a misplaced moiety, in which the optical signal from the single analyte or moiety attached thereto is increased relative to the optical signal from the misplaced moiety. In other cases, an enhanced substrate may de-amplify an optical signal from a single analyte or a moiety attached thereto and an optical signal from a misplaced moiety, in which the optical signal from the single analyte or moiety attached thereto is increased relative to the optical signal from the misplaced moiety.

An optical signal, as set forth herein, may be characterized with respect to a signal-to-noise ratio (SNR). An SNR for an optical signal may be determined with respect to a background or baseline optical signal. A background or baseline optical signal may be spatially-uniform or spatially-variant across an array, including with respect to spatial and temporal variations in said background or baseline optical signals. A single analyte, a moiety attached thereto, or a misplaced moiety may be considered to be detected if a magnitude of an optical signal from the single analyte, the moiety attached thereto, or the misplaced moiety comprises an SNR above a threshold value, such as at least about 1.25, 1.5, 2, 2.5, 3, 4, 5, 6, 7, 8, 9, 10, or more than 10. A single analyte, a moiety attached thereto, or a misplaced moiety may be considered to not be detected if a magnitude of an optical signal from the single analyte, the moiety attached thereto, or the misplaced moiety comprises an SNR below a threshold value, such as no more than about 10, 9, 8, 7, 6, 5, 4, 3, 2, 1.5, 1.25, or less than 1.25.

A method, as set forth herein, may comprise detecting presence of a first optical signal from a first single analyte or a first moiety attached thereto, and presence of a second optical signal from a second single analyte or a second moiety attached thereto, in which the first optical signal is produced at a first distance with respect to a surface of a substrate or a surface of a layered or deposited material, in which the second optical signal is produced at a second distance with respect to a surface of a substrate or a surface of a layered or deposited material, and in which the first distance and the second distance differ. In some cases, a method, as set forth herein, may further comprise detecting presence or absence of a third optical signal from a misplaced moiety, in which the third optical signal is produced at a third distance with respect to a surface of a substrate or a surface of a layered or deposited material, and in which the third distance differs from the first distance and the second distance.

A method, as set forth herein, may comprise optically detecting presence of a first signal from a first single analyte or a moiety attached thereto, or presence of a second signal from a second single analyte or a moiety attached thereto. In some cases, optical detection can be performed on an optical detection system. An optical detection system may utilize any suitable optical detection method, such as optical microscopy (e.g., fluorescence microscopy), surface plasmon resonance, infrared spectroscopy, ultraviolet spectroscopy, or a combination thereof. An optical detection system may comprise additional components, such as a light source (e.g., a laser, light-emitting diode, light bulb, etc.), a lens (e.g., a collimating lens, a focusing lens, a de-focusing lens, a polarizing lens, a filtering lens, etc.), a mirror (e.g., a reflective mirror, a dichroic mirror, etc.), and a sensor (e.g., a pixel-based array).

In some cases, a method set forth herein may comprise the steps of: i) coupling a first detectable label to a first single analyte and a second detectable label to a second single analyte; and ii) detecting presence of a first optical signal from the first detectable label at a first site of an array and presence of a second optical signal from the second detectable label at a second site the array. In some cases, a detectable label may be coupled to a single analyte before the single analyte is coupled to a site of an array. For example, a sequencing assay (e.g., an Edman-type polypeptide sequencing method) may comprise coupling a plurality of detectable labels to a single analyte, then detecting the step-wise removal of the detectable labels based upon a concomitant decrease in optical signal magnitude. In other cases, a detectable label may be coupled to a single analyte after the single analyte is coupled to an array. For example, an identification assay may comprise coupling a detectable affinity agent to a single analyte that is coupled to an array, then detecting the presence of the detectable affinity agent at a site of the array to which the single analyte is coupled. In some cases, a method set forth herein may utilize an affinity agent (e.g., an aptamer, an oligonucleotide, an antibody or antibody fragment, a protein binding agent, etc.), in which the affinity agent comprises a detectable label. In particular cases, a detectable label may comprise a fluorophore or luminophore. In some cases, a method set forth herein may further comprise removing a first detectable label from a first single analyte or a second detectable label from a second single analyte. A removing step may occur before a detection step. A removing step may occur after a detection step. A removing step can comprise a degradation reaction (e.g., an Edman-type degradation reaction). A removing step can comprise a rinsing step (e.g., stripping an affinity agent from a single analyte to which it is bound). In some cases, a method set forth herein may further comprise: i) coupling a third detectable label to an interstitial region of one or more interstitial regions of an array, and ii) detecting absence of a third signal from the third detectable label at the interstitial region. In particular cases, distance of a first detectable label coupled to a first single analyte or a moiety attached thereto may differ from distance of a second detectable label coupled to a second single analyte or a moiety attached thereto with respect to a surface of a substrate or a surface of a layered or deposited material. In particular cases, distance of a detectable label coupled to a single analyte or a moiety attached thereto may differ from distance of a third detectable label coupled to a misplaced moiety with respect to a surface of a solid support or a surface of a layered or deposited material.

A method set forth herein may utilize an array containing a plurality of single analytes, in which the plurality of single analytes is heterogeneous with respect to one or more properties. A suitable enhanced substrate for forming such an array may be characterized as producing an enhanced optical signal for an increased fraction of array sites or single analytes bound thereto relative to a non-enhanced array (i.e., an array lacking a layer or coating of a layered or deposited material). An enhanced optical signal may comprise an increased difference between a signal magnitude of an optical signal from a single analyte or a moiety attached thereto relative to an optical signal from a misplaced moiety. For a given population of single analytes that is heterogeneous with respect to one or more properties, an enhanced substrate may facilitate the detection of an enhanced signal from a fraction of sites containing a single analyte of the population of single analytes, such as at least about 5%, 10%, 20%, 30%, 40%, 50%, 60%, 70%, 80%, 90%, 95%, 99%, 99.9%, or more than 99.9% of sites. Alternatively or additionally, an enhanced substrate may facilitate the detection of an enhanced signal from a fraction of sites containing a single analyte of the population of single analytes, such as no more than about 99.9%, 99%, 95%, 90%, 80%, 70%, 60%, 50%, 40%, 30%, 20%, 10%, 5%, or less than 5% of sites.

A method set forth herein may comprise a step of coupling a plurality of single analytes to a plurality of sites of an array. In some cases, a method set forth herein may comprise a step of coupling a plurality of single analytes to a plurality of sites of an array, in which the plurality of single analytes is heterogeneous with respect to at least one property (e.g., mass, hydrodynamic radius, length, isoelectric point, analyte type, etc.). A spatial distribution of a plurality of single analytes that is heterogeneous with respect to at least one property may be random, non-random, stochastic, or deterministic. A random or stochastic spatial distribution of single analytes on an array may be formed on an enhanced substrate, in which each site of the array comprises a substantially uniform structure or surface chemistry. For example, each array site may comprise a coupling moiety that is configured to couple a single analyte, in which each single analyte of a heterogeneous plurality of single analytes comprises an equal chance of becoming coupled to an array site. A non-random or deterministic spatial distribution of single analytes on an array may be formed on an enhanced substrate, in which a first site of the array comprises a first structure or surface chemistry that is configured to bind a first type of single analyte, and in which a second site of the array comprises a second structure or surface chemistry that is configured to bind a second type of single analyte.

A method set forth herein may comprise a step of, before forming an enhanced substrate, as set forth herein, determining a first thickness and/or a second thickness of a layer or coating formed on a solid support. In some cases, a first thickness and/or a second thickness of a layer or coating disposed on a substrate may be determined empirically. For example, arrays containing pluralities of single analytes (e.g., pluralities of single analytes that are heterogeneous with respect to at least one property, homogeneous pluralities of single analytes) may be formed, in which each array is formed on an enhanced substrate with a differing configuration of layered or deposited material (e.g., differing thicknesses of layered or deposited material at array sites, differing thicknesses of layered or deposited material at interstitial regions, etc.). Detection of each array may be performed to determine which array configuration produces desired or optimal detection of single analytes (e.g., least detection of misplaced moieties, increased signal from a largest fraction of single analytes or moieties attached thereto relative to misplaced moieties, etc.). In other cases, a first thickness and/or a second thickness of a layer or coating of a layered or deposited material disposed on a substrate may be determined computationally or theoretically. Exemplary theoretical relationships for determining a thickness of a layer or coating may be found in, for example, U.S. Pat. No. 7,988,918B2 and Lambacher, et al., Appl. Phys. A, vol. 63 (2000), each of which is herein incorporated by reference.

A relative magnitude of amplification or de-amplification of an optical signal detected on an enhanced substrate may be related to distance of an optical signal source from a surface of a substrate or a surface of a layered or deposited material disposed on the substrate. Accordingly, a single-analyte assay or process performed on an array comprising a plurality of single analytes that is heterogeneous with respect to one or more properties (e.g., analyte size, analyte length, analyte structure, etc.) may comprise an associated variability in location of optical signal sources for the plurality of single analytes. For example, FIG. 2 depicts an array comprising a plurality of single analytes, in which the plurality of single analytes is heterogeneous in length. Accordingly, terminal optical signal sources 241 for each single analyte are located at differing distances from the upward facing surface of the substrate 200. In some configurations, an enhanced substrate may be structured to decrease a variability in location of optical signal sources associated with single analytes. In other configurations, analytes may be coupled to an array in a manner that decreases variability in location of optical signal sources relative to the enhanced substrate.

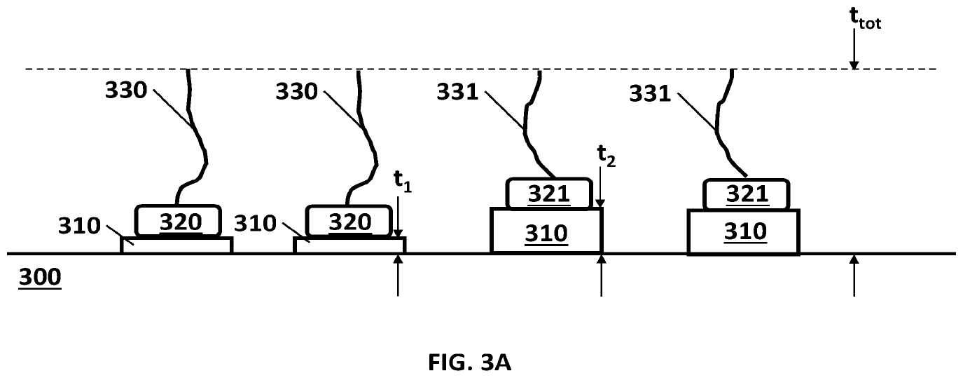

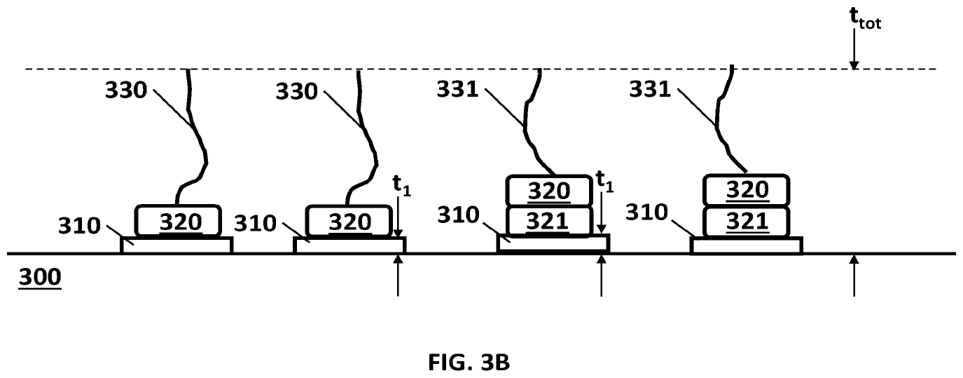

FIGS. 3A-3B depict cross-sectional views of alternative array configurations for arrays comprising pluralities of single analytes of varying sizes. Referring to FIG. 3A, an array comprises a substrate 300 with raised features comprising a layered or deposited material 310. The array comprises a first region containing raised features of a lesser thickness, t₁, and a second region containing raised features of a greater thickness, t₂. An upper surface of each raised feature couples an anchoring moiety (320, 321) that couples a single analyte (330, 331) to the raised feature. Anchoring moieties 320 couple larger single analytes 330 to raised features in the first region. Anchoring moieties 321 couple smaller single analytes 331 to raised features in the second region. The difference in thickness between raised features in the first region and raised features in the second region facilitates a reduction in variability of total distance, tₜₒₜ, for the maximum extent of each single analyte from the surface of the enhanced substrate. Referring to FIG. 3B, an array is depicted with a similar configuration to FIG. 3A, but each raised feature comprises substantially the same thickness. To decrease variability in total distance, tₜₒₜ, of maximum extent of single analytes from the surface of the enhanced substrate, differing configurations of anchoring moieties (320, 321) are utilized. For larger single analytes 330, only a single anchoring moiety is utilized. For shorter single analytes 331, anchoring moieties (320 and 321) are stacked to raise the single analytes 331 further from the surface. Alternatively, larger or differently shaped anchoring moieties may be utilized to achieve the same effect. For example, single analytes may be coupled to sites of an array by anchoring moieties comprising nucleic acid origami. For smaller single analytes, nucleic acid origami may be designed to modularly bind and stack to each other to raise single analytes away from the array surface. In some cases, a method of forming an array of single analytes may comprise fractionating single analytes according to a property in which the single analytes are heterogeneous (e.g., weight, hydrodynamic radius, length, isoelectric point, etc.). Separation of analytes may be achieved by any suitable method, such as liquid chromatography, size exclusion chromatography, affinity chromatography, ultrafiltration, tangential flow filtration, centrifugation, or a combination thereof. The analytes can be separated according to any of a variety of characteristics such as differences in molecular size, molecular weight, polymer length, mass, charge, pKA, hydrodynamic radius, polarity, hydrophobicity, hydrophilicity, or the like. In particular cases, a method of forming an array may further comprise coupling each single analyte of a unique fraction of separated single analytes to an anchoring moiety that is specific to that fraction of single analytes. For example, FIG. 3A depicts use of differing nucleic acid anchoring moieties (320, 321) for different lengths of single analyte (330, 331, respectively). In some cases, a site of an array may be configured to bind analytes of a specific fraction of separated single analytes (e.g., comprising a surface-linked coupling group that is configured to couple a particular fraction of single analytes or anchoring moieties).

A method, as set forth herein, may comprise a step of determining an optimal thickness of a layer disposed upon a substrate. The optimal thickness may be determined based upon the optical properties of a system, including the stimulation wavelength of a fluorophore, the emission wavelength of a fluorophore, and the indexes of refraction of materials of the array (e.g., a substrate, a layer disposed upon the substrate). An optimal thickness of a layer disposed upon a substrate may be determined to reduce optical signals from a signal source that is known to non-specifically bind to a surface of an array or substrate (e.g., non-specific binding of a fluorophore or other detectable moiety). In the specific case of a multispectral system (e.g., a system utilizing two or more signal sources), an optimal thickness of a layer disposed on a substrate may be chosen based upon: i) a layer thickness that facilitates reduced signal from a signal source with a larger quantity of non-specific binding, or ii) a layer thickness that produces the greatest reduction of signal from both signal sources (i.e., a maximum overall reduction in unwanted optical signals).

In an aspect, provided herein is a composition, comprising: a) a solid support, b) a layer disposed upon the substrate, wherein the layer comprises raised features of a first average thickness and indented features of a second average thickness, c) a plurality of anchoring moieties coupled to the layer, and d) a plurality of single analytes, wherein each single analyte is coupled to one and only one anchoring moiety of the plurality of anchoring moieties. In some configurations, each raised feature may comprise an array site. In other configurations, each indented feature may comprise an array site. In some configurations, each raised feature may comprise an interstitial region. In other configurations, each indented feature may comprise an interstitial region.

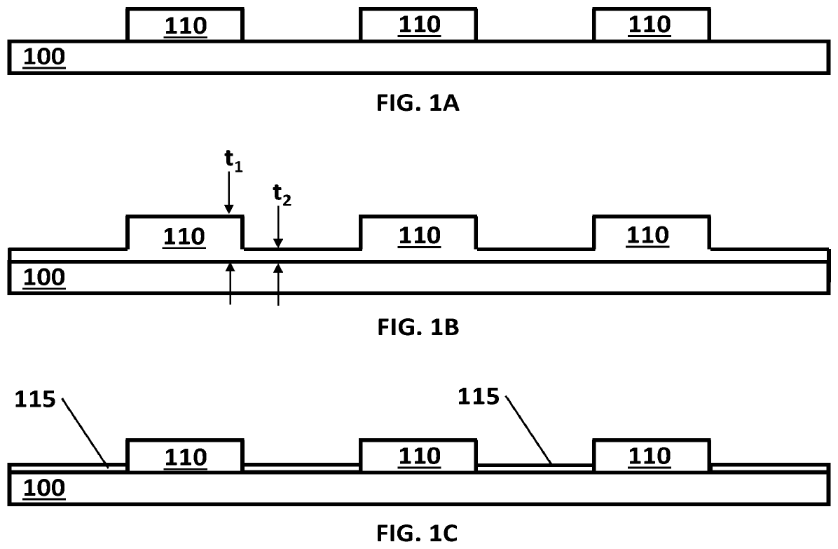

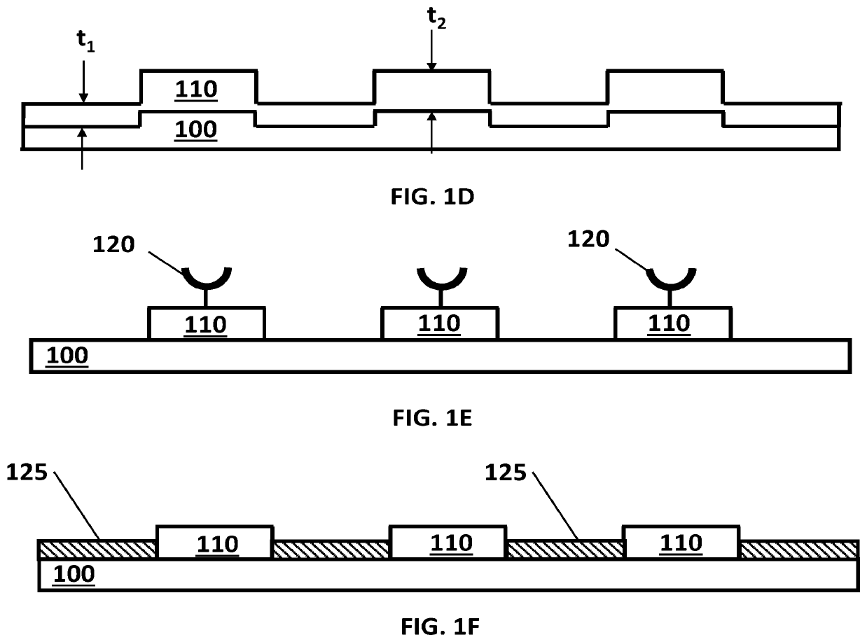

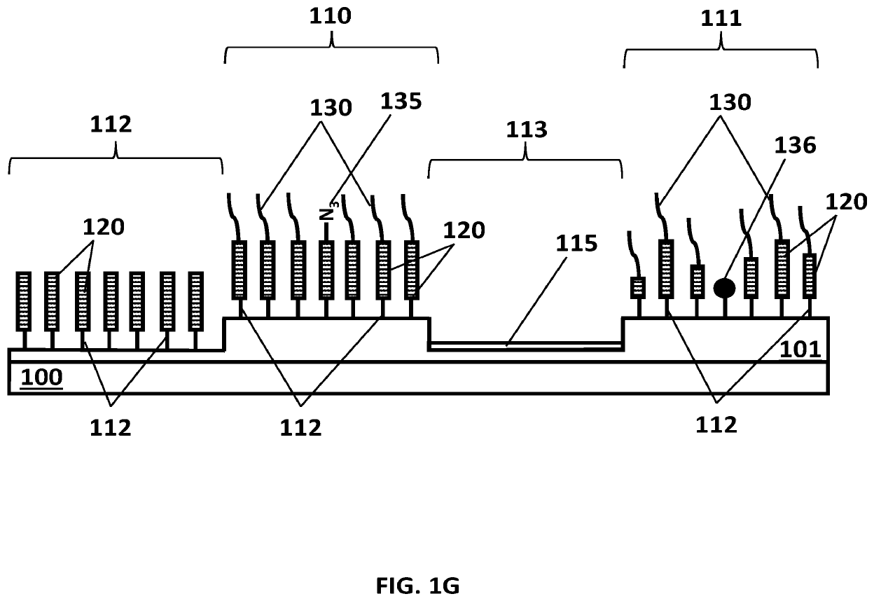

FIGS. 1A-1G depict cross-sectional views of useful configurations of substrates for enhanced optical detection. The depicted substrates may be useful for forming arrays of single analytes, as set forth herein. Each substrate comprises a substrate 100 with one or more layered or deposited materials 110 disposed adjacent to the substrate 100. Referring to FIG. 1A, a substantially planar substrate comprises a plurality of raised features comprising a layered or deposited material 110. In the depicted configuration, each raised feature comprises a substantially planar upper face that is parallel to a surface of the substrate 100 upon which the layered or deposited material 110 is disposed. Indented features between the raised features contain exposed areas of the surface of the substrate 100. In some configurations, the upper faces of the raised features may comprise binding sites for the coupling of single analytes. In other configurations, the indented features between raised features may comprise binding sites for the coupling of single analytes. Referring to FIG. 1B, the depicted substrate comprises a similar configuration to the substrate depicted in FIG. 1A, but with a continuous or semi-continuous coating of the layered or deposited material 110 on the surface of the substrate 100. The layered or deposited material comprises an average or maximal thickness, t₂, at the raised features, and an average or minimal thickness, t₁, at the indented features. Referring to FIG. 1C, the depicted substrate comprises a similar configuration to the substrate of FIG. 1B, but with two differing materials disposed on the surface of the substrate 100. The raised features comprise a first layered material 110 and the indented features comprise a second layered material 115. The first layered material 110 and the second layered material 115 may differ with respect to one or more chemical properties, such as composition, index of refraction, density, reactivity, etc. Referring to FIG. 1D, the depicted substrate comprises a similar configuration to FIG. 1B, but with the layered or deposited material 110 applied to a non-planar surface of the substrate 100. In some configurations, the non-planar surface of the substrate 100 may be formed by a lithographic process prior to the deposition of the layered or deposited material 110. The coating of layered or deposited material 110 may comprise a spatially-variable thickness on the substrate 100, such as the depicted minimum thickness, t₁, at the indented features, and the depicted maximum thickness, t₂, at the raised features. As shown in FIGS. 1E-1F, the depicted substrates comprise a similar configuration to FIG. 1A, but with additional moieties added to a surface of the substrate 100 or the layered or deposited material 110. Referring to FIG. 1E, the depicted substrate comprises raised features containing a layered or deposited material 110. The raised features comprises a substantially planar upper face that comprises a moiety 120 that is configured to bind an analyte to the surface (e.g., a covalent coupling moiety, a non-covalent coupling moiety, etc.). In a first alternative embodiment, the moiety 120 that is configured to bind an analyte to the surface may be provided to a surface of the substrate 100 in an indented feature of the substrate. In a second alternative embodiment, the moiety 120 that is configured to bind an analyte to the surface may be provided to surfaces of both the indented features and the raised features. Referring to FIG. 1F, the depicted substrate comprises indented features that comprise a passivating moiety 125 or a layer of passivating moieties 125 that are configured to inhibit binding of unbound moieties to a surface of the substrate. In a first alternative embodiment, the passivating moiety 125 or the layer of passivating moieties 125 that are configured to inhibit binding of unbound moieties to a surface of the substrate may be provided to an upper surface of the raised features. In a second alternative embodiment, the passivating moiety 125 or the layer of passivating moieties 125 that are configured to inhibit binding of unbound moieties to a surface of the substrate may be provided to a surface of the raised features and a surface of the indented features.

FIG. 1G illustrates additional aspects of array site and interstitial region structuring and/or surface chemistry. FIG. 1G depicts a cross-sectional view of a solid support comprising a substrate 100, an optional layer or material 101 patterned on the substrate 100 to form array sites 110 and 111, and interstitial regions 112 and 113. The surface chemistries of array sites 110 and 111 differ structurally, and the surface chemistries of interstitial regions 112 and 113 also differ structurally. Array site 110 comprises a plurality of moieties coupled to a surface (e.g., a surface of the layer or material 101). Each moiety comprises a surface-coupling moiety 112, an optional spacing or passivating moiety 120, and an optional coupling moiety (e.g., an oligonucleotide 130 or a covalent bond-forming group 135). The optional spacing or passivating moieties 120 are substantially homogeneous (e.g., with respect to length, molecular weight, degree of branching, etc.). Accordingly, array site 110 has a substantially layered structure, with a spacing or passivating layer comprising the spacing or passivating moieties 120 closer to the surface, and a coupling layer comprising the coupling moieties 130 and 135 further from the surface. Array site 111 has a similar structure to array site 110, with several differences. Optional spacing or passivating moieties 120 vary with respect to one or more characteristics (e.g., length, molecular weight, degree of branching, net electrical charge, chemical structure, etc.). Further, a coupling moiety 136 (e.g., a component of a receptor-ligand binding pair) is coupled to the surface of the optional layer or material 101 by a surface-coupling moiety 112 but does not have a spacing or passivating moiety 120. Surface chemistries of array sites may be varied (e.g., like array sites 110 and 111) to 1) create orthogonal binding characteristics, thereby permitting specific coupling of differing analytes and/or anchoring groups at appropriate array sites, and/or 2) control or vary binding kinetics of analytes and/or anchoring groups at array sites. Additional aspects of array site configurations are described in U.S. Pat. Nos. 11,203,612B2 and 11,505,796B2, and U.S. Patent Publication No. 20220379582A1, each of which is herein incorporated by reference in its entirety.

Continuing with FIG. 1G, interstitial region 112 comprises a plurality of moieties that are coupled to a surface of the layer or material 101. Each moiety may comprise a surface coupling moiety 112 and a spacing or passivating moiety 120 (e.g., a hydrophobic polymer, a hydrophilic polymer, a branched polymer, a linear polymer, an electrically-charge polymer, a zwitterionic polymer, etc.). Optionally, the spacing or passivating moieties 120 may be homogeneous or heterogeneous with respect to one or more properties (e.g., length, molecular weight, degree of branching, net electrical charge, chemical structure, etc.). Interstitial region 113 comprises a layer or coating 115 that is disposed on the surface of the optional layer or material 101. The layer or coating 115 may comprise a material with an adhesion-inhibiting chemical characteristic (e.g., hydrophobicity, electrical-charge, steric occlusion, etc.). For example, a hydrophobic photoresist adhesion promoter (e.g., HMDS) may sufficiently inhibit adhesion of analytes, anchoring groups, or detectable probes to a surface of an interstitial region. Methods of forming arrays are described in more detail in U.S. Pat. Nos. 11,203,612B2 and 11,505,796B2, each of which is herein incorporated by reference in its entirety.

An enhanced substrate, as set forth herein, may comprise a layered or deposited material disposed on a substrate or a surface thereof. A layered or deposited material may be disposed on a substrate or a surface thereof in a layer or coating. The layered or deposited material may comprise any suitable material, such as a metal, metal oxide, a dielectric material, or a combination thereof. A metal may include any suitable metal, including Si, Ge, Al, Cu, Au, Ag, Ti, W, Fe, Ni, Mo, Mn, and combinations thereof. A metal oxide may include any suitable metal, including Al₂O₃, iron oxides, SiO₂, TiO₂, Ta₂O₅, HfO₂, ZrO₂, MgO, and combinations thereof. A dielectric material may any suitable dielectric material, including SiO₂, TiO₂, Ta₂O₅, HfO₂, ZrO₂, MgO, Si₃N₄, MgF₂ and YF₃. In some configurations, an enhanced substrate may comprise two or more layers of layered or deposited material. In particular configurations, an enhanced substrate may comprise two or more layers of layered or deposited material, in which a first layer comprises a first material and a second layer may comprise a second material, and in which the first material differs from the second material (e.g., a first dielectric material and a differing second dielectric material). A layered or deposited material may be disposed on a substrate by any suitable method, such as atomic layer deposition, chemical vapor deposition, chemical liquid deposition, or a combination thereof.

Methods for forming solid supports, as set forth herein, are known in the art. Suitable methods may include lithographic methods for patterning substrates and/or layered or deposited materials. Deposition of a layered or deposited material on a substrate may occur before lithographic patterning or during lithographic patterning (e.g., deposition of the layered or deposited material at regions of a substrate where a resist material has been removed by lithography). For certain array configurations, it may be useful to provide array sites with orthogonal binding chemistries. For example, two array sites may be provided at an optically non-resolvable distance, in which a first array site of the two array sites is configured to bind an analyte with a first immobilized avidity component, and in which a second array site of the two array sites is configured to bind an analyte with a second immobilized avidity component that differs from the first immobilized avidity component. Accordingly, it may be useful to provide the first array site with an analyte-binding chemistry that differs from that of the second array site. In another example, analytes from differing samples may be multiplexed on a single array if the array contains two differing sets of array sites that are distinguished by their respective analyte-binding chemistries. Array sites may be formed with orthogonal binding and/or detection characteristics by varying a thickness of a layered or deposited material between differing array sites. Thickness of a layered or deposited material may be varied by: 1) lithography patterning of the layered or deposited material, and/or 2) deposition conditions when forming a layer of the layered or deposited material. Array sites may be formed with orthogonal binding and/or detection characteristics by providing sites with differing surface chemistries. Sites with differing surface chemistries may be formed by sequential lithographic patterning and surface chemistry deposition. Additionally, sites with differing surface chemistries may be formed by providing different surface materials at each site. For example, a first array site may be provided with an SiO₂ surface and organosilane surface-coupling moieties attached thereto, and a second array site may be provided with a ZrO₂ surface and organophosphate or organophosphonate surface-coupling moieties attached thereto. Techniques for forming arrays are described in more detail in U.S. Pat. Nos. 11,203,612 and 11,505,796 and U.S. patent application Ser. No. 18/192,606 each of which is herein incorporated by reference in its entirety.

A composition for a layered or deposited material may be selected based upon one or more optical properties. Selection of materials for forming an enhanced substrate (i.e., selection of a solid support material and a layered or deposited material) can be influenced by the nature of the optical interaction that is to be detected by an optical detection system (e.g., fluorescence, emission, absorption, reflection, refraction, etc.). In the specific case of a fluorescent or luminescent system, material choice may be influenced by excitation wavelength and/or emission wavelength of light within an optical detection system. In some cases, a substrate material and a layered or deposited material may be selected, in which an index of refraction of the substrate is larger than an index of refraction of the layered or deposited material. A substrate material and a layered or deposited material may be selected, in which a difference in index of refraction between the substrate material and the layered or deposited material is at least about 0.1, 0.5, 1.0, 1.5, 1.6, 1.7, 1.8, 1.9, 2.0, 2.1, 2.2, 2.3, 2.4, 2.5, or greater than 2.5, Alternatively or additionally, a substrate material and a layered or deposited material may be selected, in which a difference in index of refraction between the substrate material and the layered or deposited material is no more than about 2.5, 2.4, 2.3, 2.2, 2.1, 2.0, 1.9, 1.8, 1.7, 1.6, 1.5, 1.0, 0.5, 0.1, or less than 0.1.

A composition of the present disclosure may comprise a single-analyte array, in which the single-analyte array comprises a plurality of sites. A single-analyte array may comprise a plurality of sites, in which each site of the plurality of sites comprises a single analyte that is coupled to the site, and in which each site comprises no more than one single analyte coupled to the site. In some configurations, a single-analyte array may comprise one or more array sites that comprise no single analytes. In some configurations, a single-analyte array may comprise one or more array sites that comprise more than one single analyte. A single-analyte array may be characterized as comprising a fraction of sites containing one and only single analyte that exceeds a fraction predicted by a Poisson distribution, such as at least about 40%, 50%, 60%, 70%, 80%, 90%, 95%, 99%, 99.9%, or more than 99.9% of array sites.

A single-analyte array composition may comprise a plurality of anchoring moieties (e.g., nucleic acids, structured nucleic acid particles, nanoparticles, etc.), in which each anchoring moiety of the plurality of anchoring moieties is configured to couple a single analyte to a site of the single-analyte array. In some configurations, each anchoring moiety of the plurality of anchoring moieties may be coupled to a single raised feature. In other configurations, each anchoring moiety of the plurality of anchoring moieties may be coupled to a single indented feature. In some configurations, an array site may comprise two or more anchoring moieties (e.g., structured nucleic acid particles, etc.) and one and only one single analyte. In particular configurations, an array site may comprise two or more anchoring moieties, in which an anchoring moiety of the two or more anchoring moieties is coupled to a second anchoring moiety of the two or more anchoring moieties, and is further coupled to a single analyte.

In some configurations, a single-analyte array composition may comprise a layer or coating containing a layered or deposited material, in which an anchoring moiety of the plurality of anchoring moieties is covalently coupled to the layer or coating. For example, a nucleic acid may be covalently attached to a layer or coating comprising a layered or deposited material by a click-type reaction (e.g., a reaction of dibenzocyclooctylene with azide, a reaction of methyltetrazine with transcyclooctene, etc.). In other configurations, a single-analyte array composition may comprise a layer or coating containing a layered or deposited material, in which an anchoring moiety of the plurality of anchoring moieties is non-covalently coupled to the layer or coating. For example, a nucleic acid may be adsorbed by an electrostatic interaction, a nucleic acid hybridization reaction, or a ligand-receptor binding interaction (e.g., streptavidin-biotin, SpyCatcher-Spytag, etc.).

In some configurations, a single-analyte array composition may comprise a plurality of single analytes, in which one or more single analyte(s) of the plurality of single analytes is/are covalently coupled to an anchoring moiety of the plurality of anchoring moieties. For example, a single analyte may be covalently attached to a nucleic acid by a click-type reaction (e.g., a reaction of dibenzocyclooctylene with azide, a reaction of methyltetrazine with transcyclooctene, etc.). In other configurations, a single-analyte array composition may comprise a plurality of single analytes, in which one or more single analyte(s) of the plurality of single analytes is non-covalently coupled to an anchoring moiety of the plurality of anchoring moieties. For example, a single analyte may be coupled by an electrostatic interaction, a nucleic acid hybridization reaction, or a ligand-receptor binding interaction (e.g., streptavidin-biotin, SpyCatcher-Spytag, etc.). A single-analyte array composition may comprise one or more single-analyte(s) that is/are not coupled to an array site by an anchoring moiety (e.g., a nucleic acid, a nanoparticle). In some configurations, a single analyte may be directly coupled to a site by a covalent interaction. In other configurations, a single analyte may be directly coupled to a site by a non-covalent interaction.

A layered or deposited material may be deposited on a substrate of an enhanced substrate, in which thickness of a layer or coating of the layered or deposited material varies between sites of the array and interstitial regions that separate array sites from other array sites. In some cases, thickness of a layer or coating of a layered or deposited material may be thicker at sites of an array and thinner at interstitial regions. In other cases, thickness of a layer or coating of a layered or deposited material may be thicker at interstitial regions and thinner at sites of an array. In some cases, thickness of a layer of a layered or deposited material at a site or an interstitial region may be about 0 nanometers (i.e., no layered or deposited material). In other cases, a thickness of a layer of a layered or deposited material at a site or an interstitial region may be greater than about 0.1 nanometers (i.e., layered or deposited material with a measurable thickness). A layered or deposited material may have a thickness of at least about 1 nanometer (nm), 5 nm, 10 nm, 25 nm, 50 nm, 100 nm, 150 nm, 200 nm, 250 nm, 300 nm, 400 nm, 500 nm, 600 nm, 700 nm, 800 nm, 900 nm, 1000 nm, or greater than 1000 nm. Alternatively or additionally, a layered or deposited material may have a thickness of no more than about 1000 nm, 900 nm, 800 nm, 700 nm, 600 nm, 500 nm, 400 nm, 300 nm, 250 nm, 200 nm, 150 nm, 100 nm, 50 nm, 25 nm, 10 nm, 5 nm, 1 nm, or less than 1 nm. A layer thickness may be determined to not exceed a thickness beyond which an optical detection system becomes unresolved (e.g., a thickness that exceeds a depth of field for an optical detection system). For example, a microscope system may be incapable of resolving a first object and a second object when a focal plane of the first object is separated from a focal plane of the second object by 500 nanometers or more. In some cases, a maximum layer thickness may be determined when including a dimension of any additional objects bound to a surface of an array, as set forth herein, such as analytes, anchoring moieties, and signal sources (e.g., detectable labels, affinity agents, etc.). For example, a maximum layer thickness may be determined as the difference between the maximum depth-of-field of a microscope less the average thickness of anchoring groups and the average thickness of analytes coupled to each anchoring group.

An enhanced substrate, as set forth herein, may comprise one or more surface-linked moieties. A surface-linked moiety may comprise a passivating moiety. A passivating moiety may comprise a moiety or functional group that is configured to inhibit binding of an unbound moiety to an enhanced substrate or a surface thereof. Exemplary passivating moieties may comprise surface-linked polymers such as polyethylene glycol, alkanes, fluorinated alkanes, dextrans, dendrimers, branched versions thereof, or combinations thereof. Passivating moieties are described in more detail, for example in Patent Cooperation Treaty Publication No. WO 2021087402A1 and U.S. Pat. No. 11,505,796B2, each of which is herein incorporated by reference. In some cases, a passivating moiety may be coupled to a solid support or a layered or deposited material. In some cases, a passivating moiety may be coupled at an interstitial region of an enhanced substrate. In other cases, a passivating moiety may be coupled at a site of an array. In some cases, array sites and interstitial regions may comprise passivating moieties.

Arrays with Increased Site Density

Further provided herein are arrays with an increased surface density of array sites. For some assays, including assays performed with single-analyte resolution, it may be preferable to provide as many resolvable analytes as possible on an array. However, conventional microscope systems utilizing common detectable labels (e.g., visible or near-infrared fluorescent labels) are typically diffraction-limited. Accordingly, the optical limitations of the microscope/label system limit the achievable array density, or more advanced microscopic techniques can be utilized, often at the cost of decreased throughput of analytes (i.e., fewer analytes scanned per unit time, or longer overall scan times for an array). The array configurations provided herein may contain sites with spacings less than the minimum spacing for optical resolution of each site on a diffraction-limited detection device. Further, methods are provided for detecting signals from each array site of the described high-density arrays.

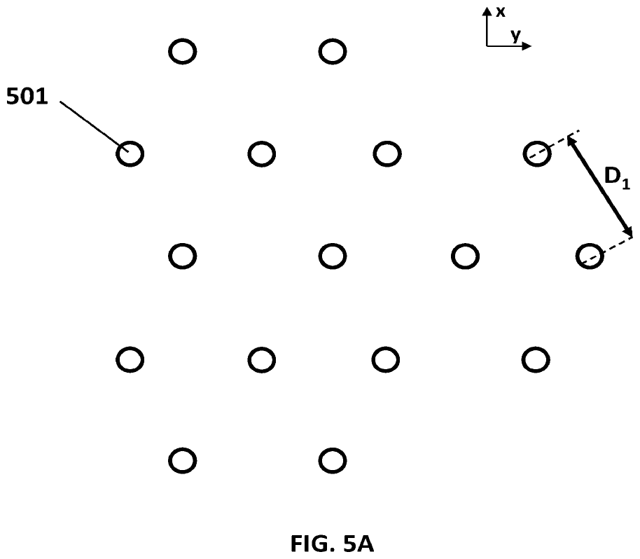

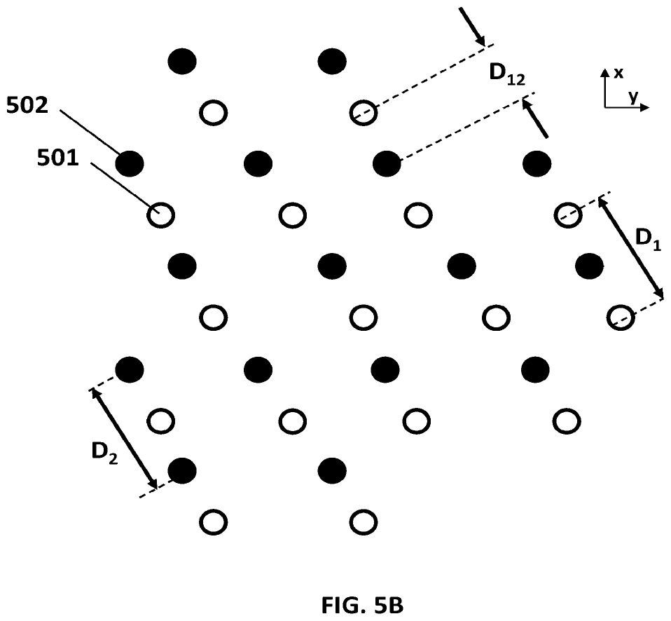

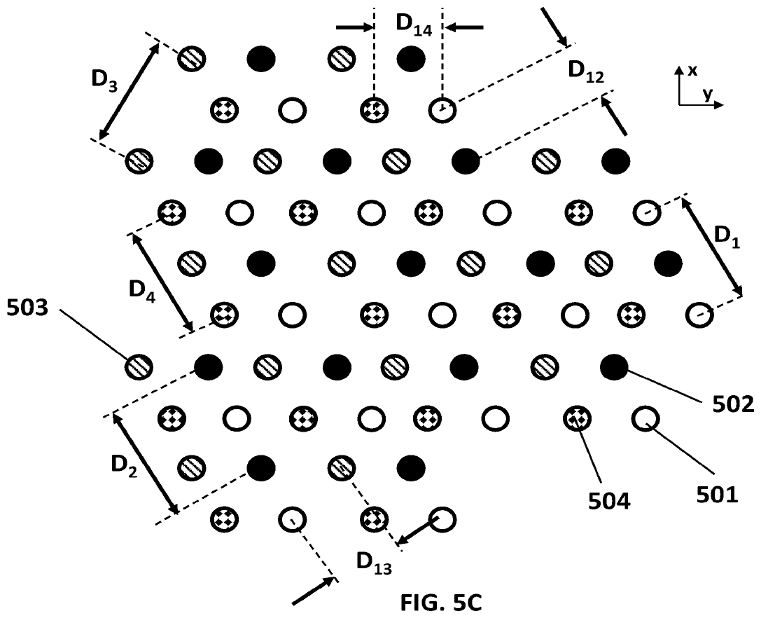

FIGS. 5A-5C illustrate aspects of high-density array configurations. FIG. 5A depicts a plurality of array sites 501, in which the sites 501 have a regular or patterned hexagonal arrangement. The sites 501 have an average or minimum pitch D₁, optionally such that each site 501 is optically resolvable. FIG. 5B depicts an array configuration with increase site density. The array comprises the first plurality of sites 502 (as depicted in FIG. 5A) and a second plurality of sites 502 that also have a regular or patterned hexagonal arrangement. The first plurality of sites 501 has an average or minimum pitch D₁ and the second plurality of sites 502 has an average or minimum pitch D₂. In some configurations, pitches D₁ and D₂ may be substantially equal. Optionally, each site 501 is optically resolvable from each other site 501 of the first plurality of sites 501, and each site 502 is optically resolvable from each other site 502 of the second plurality of sites 502. Optionally, each site 501 of the first plurality of sites 501 may have a pitch D₁₂ with respect to an adjacent site 502 of the second plurality of sites 502 that is not optically resolvable. FIG. 5C depicts a doubling of the site density relative to FIG. 5B by including a third plurality of sites 503 and a fourth plurality of sites 504. The first plurality of sites 501 has an average or minimum pitch D₁, the second plurality of sites 502 has an average or minimum pitch D₂, the third plurality of sites 503 has an average or minimum pitch D₃, and the fourth plurality of sites 504 has an average or minimum pitch D₄. In some configurations, pitches D₁, D₂, D₃, and/or D₄ may be substantially equal. Optionally, each site 501 is optically resolvable from each other site 501 of the first plurality of sites 501, each site 502 is optically resolvable from each other site 502 of the second plurality of sites 502, each site 503 is optically resolvable from each other site 503 of the third plurality of sites 503, and each site 504 is optically resolvable from each other site 504 of the fourth plurality of sites 504. Optionally, each site 501 of the first plurality of sites 501 may have pitches D₁₂, D₁₃, and/or D₁₄ with respect to an adjacent site 502, 503, and/or 504, respectively, that is not optically resolvable. A site 501 of a first plurality of sites 501 may differ from a site 502, 503, and/or 504 with respect to a surface chemistry (e.g., a presence or absence of a particular coupling moiety and/or a particular passivating moiety). A site 501 of a first plurality of sites 501 may differ from a site 502, 503, and/or 504 with respect to a binding specificity for an analyte and/or anchoring group, as set forth herein.

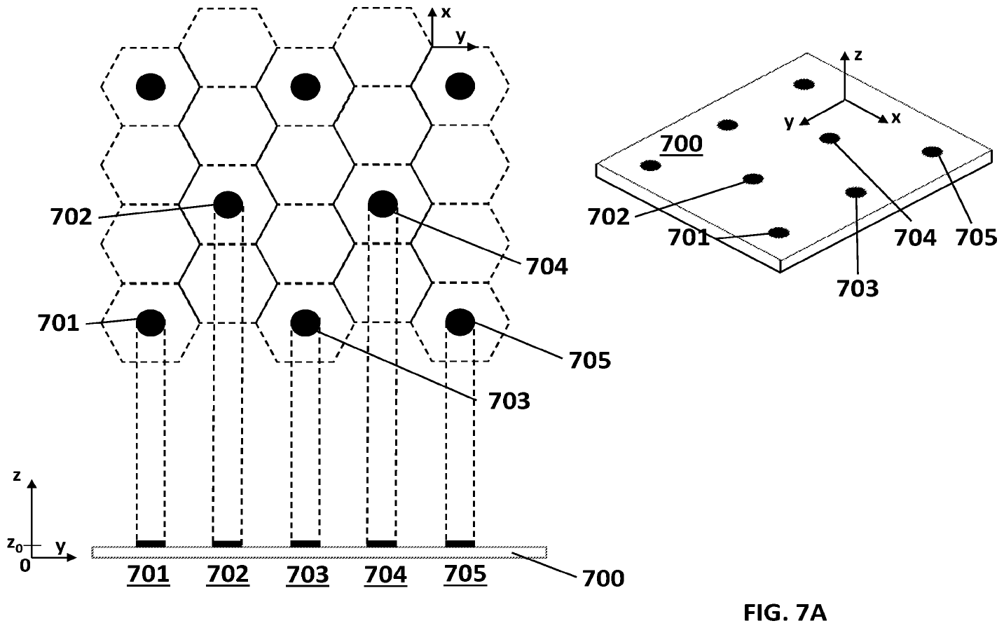

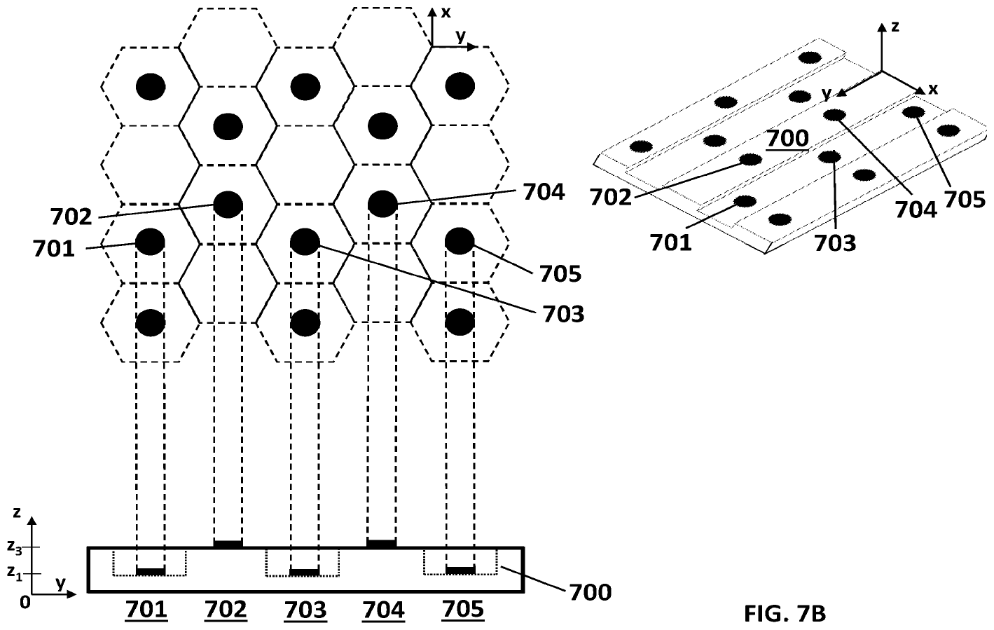

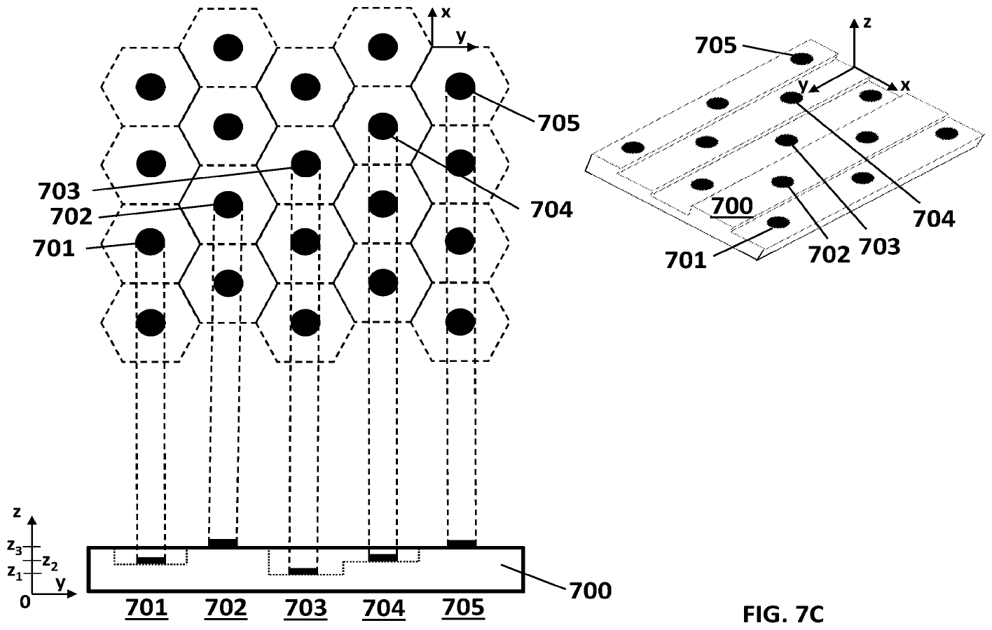

FIGS. 7A-7C depict additional high-density array configurations. FIG. 7A depicts an exploded view of an array with a similar site configuration to FIG. 5A. As shown in the upper left side of FIG. 7A, a first plurality of sites are disposed on a substrate 700 in a hexagonal configuration. The cross-sectional view (lower left) of the array shows sites 701, 702, 703, 704, and 705 disposed on a substantially planar surface of the substrate 700 at a z-axis distance of z₀ relative to a distal surface of the substrate 700. In some cases, the pitch between a site and a nearest or adjacent site may be optically resolvable. The surface of the substrate 700 may be considered a contiguous surface because any array site disposed on the surface can be reached from any other array site without crossing any breaks or discontinuities of the surface. Alternatively, the surface of substrate 700 may be considered a contiguous surface because each array site of the plurality of array sites (e.g., 701, 702, 703, 704, and 705) is disposed on a single surface. FIG. 7B depicts an exploded view of an array with an increased site density relative to the array of FIG. 7A, in which the array comprises a second plurality of sites, and in which the second plurality of sites is disposed at a different z-axis distance relative to the first plurality of sites shown in FIG. 7A. The isometric view (upper right) of FIG. 7B depicts placement of sites of the first plurality of sites (e.g., 702, 704) along ridges of the substrate 700, and placement of sites of the second plurality of sites (e.g., 701, 703, and 705) within channels or depressions of the substrate 700. The cross-sectional view (lower left) of FIG. 7B shows sites of the first plurality of sites (e.g., 702, 704) disposed at a z-axis height of z₃ relative to a distal surface of the substrate 700, and sites of the second plurality of sites disposed at a lower z-axis height of z₁ relative to a distal surface of the substrate 700. FIG. 7C depicts an exploded view of an array with an increased site density relative to the array of FIG. 7A or 7B, in which the array comprises a third plurality of sites, and in which the third plurality of sites is disposed at a different z-axis distance relative to the first plurality of sites and the second plurality of sites shown in FIG. 7B. The isometric view (upper right) of FIG. 7C depicts placement of sites of the first plurality of sites (e.g., 702, 705) along upper terraces of the substrate 700, placement of sites of the second plurality of sites (e.g., 701, 704) along middle terraces of the substrate 700, and placement of sites of the third plurality of sites (e.g., 703) along lower terraces of the substrate 700. The cross-sectional view (lower left) of FIG. 7C shows sites of the first plurality of sites (e.g., 702, 705) disposed at a z-axis height of z₃ relative to a distal surface of the substrate 700, sites of the second plurality of sites (e.g., 701, 704) disposed at a z-axis height of z₂ relative to a distal surface of the substrate 700, and sites of the third plurality of sites (e.g., 703) disposed at a z-axis height of z₁ relative to a distal surface of the substrate 700. In FIGS. 7B-7C, the surface(s) of the substrate 700 at height(s) z=z₁, z₂, and/or z₃ may be considered a non-contiguous surface because traversing from certain array sites to certain other array sites at same z-axis heights can require moving across a break or discontinuity between surfaces containing the array sites. Alternatively, the surface of substrate 700 may be considered a contiguous surface because each array site of the plurality of array sites at a particular z-axis height are not disposed on a single surface.

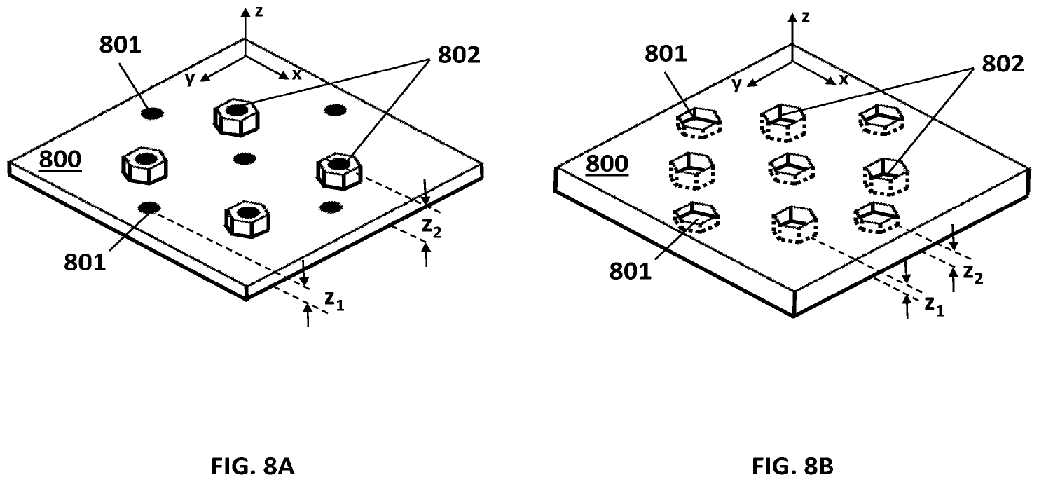

FIGS. 7B-7C depict examples of arrays that dispose sets of sites within channels or depressions of a substrate 700. In other cases, array sites may be disposed at isolated locations of an array (i.e., within or on an array feature that has no other sites). FIGS. 8A-8B illustrate array configurations with sites located at isolated locations. FIG. 8A depicts a substrate 800 with a first plurality of sites 801 disposed on a first surface of the substrate 800 (e.g., a surface having a height of z₁ relative to a distal surface of the substrate 800), and a second plurality of sites 802, in which each individual site 802 of the second plurality of sites is disposed on an individual raised feature. The raised features of FIG. 8A may each have a surface on which a site 802 is disposed, in which the surface is one or more of: i) substantially coplanar with a surface of another raised feature containing a site 802 (e.g., each surface having a height of z₂ relative to a distal surface of the substrate 800), and ii) substantially parallel to a surface containing a first plurality of sites 801. In some cases, a raised feature may contain a layered or deposited material as set forth herein. FIG. 8B depicts an array configuration in which each array site is disposed in a depression. The array may comprise a first plurality of sites 801, in which each depression of a first set of depressions comprises an individual site 801 of the first plurality of sites 801, and in which each depression of a second set of depressions comprises an individual site 802 of the second plurality of sites 802. Accordingly, each site 801 of the first plurality of sites 801 may be disposed at a z-axis height of z₂ relative to a distal surface of the substrate 800, and each site 802 of the second plurality of sites 802 may be disposed at a z-axis height of z₁ relative to a distal surface of the substrate 800.

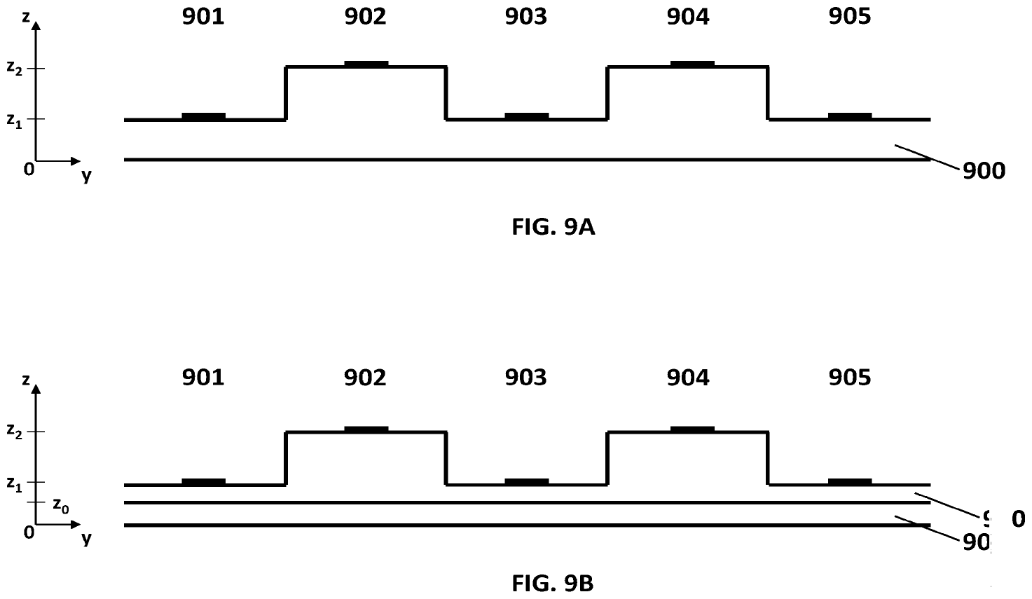

FIGS. 9A and 9B illustrate high-density array configurations with and without a layered or deposited material. FIGS. 9A and 9B depicts a solid support containing a substrate 900 with a first plurality of sites (e.g., sites 901, 903, 905 disposed at a height of z₁ relative to a distal surface of the substrate 900) disposed in depressed features and a second plurality of sites (e.g., sites 902 and 904 disposed at a height of z₂ relative to a distal surface of the substrate 900) disposed on raised features. In the configuration of FIG. 9A, the substrate 900 has been formed (e.g., lithographically) to provide the depressed features and raised features. In the configuration of FIG. 9B, a layered or deposited material 910 has been disposed on a proximal surface of the substrate 900, in which the layered or deposited material 910 has been formed (e.g., lithographically) to provide the depressed features and raised features

With respect to an array configuration, such as those depicted in FIGS. 5A-5C or FIGS. 7A-7C, the skilled person will recognize that a length scale of optical resolvability will depend upon several aspects of optical system design, including optical parameters of a detection device (e.g., magnification, numerical aperture, sensor pixel pitch, sensor pixel quantum efficiency) and detectable label optical parameters (e.g., emission wavelength, quantum efficiency, luminescence lifetime, etc.). Accordingly, a minimum length scale for optical resolvability will vary according to the design of the optical system. In some cases, a plurality of sites may have a pitch (e.g., an average pitch, minimum pitch, or maximum pitch) of at least about 10 nanometers (nm), 50 nm, 100 nm, 200 nm, 250 nm, 300 nm, 400 nm, 500 nm, 600 nm, 700 nm, 800 nm, 900 nm, 1 micron (μm), 1.1 μm, 1.2 μm, 1.3 μm, 1.4 μm, 1.5 μm, 2 μm, 3 μm, 5 μm, 10 μm, or more than 10 μm. Alternatively or additionally, a plurality of sites may have a pitch of no more than about 10 μm, 5 μm, 3 μm, 2 μm, 1.5 μm, 1.4 μm, 1.3 μm, 1.2 μm, 1.1 μm, 1 μm, 900 nm, 800 nm, 700 nm, 600 nm, 500 nm, 400 nm, 300 nm, 250 nm, 200 nm, 100 nm, 50 nm, 10 nm, or less than 10 nm. In some cases, sites of a first plurality of sites may have a pitch (e.g., an average pitch, minimum pitch, or maximum pitch) with respect to sites of a second plurality of sites of at least about 10 nm, 50 nm, 100 nm, 200 nm, 250 nm, 300 nm, 400 nm, 500 nm, 600 nm, 700 nm, 800 nm, 900 nm, 1 μm, 1.1 μm, 1.2 μm, 1.3 μm, 1.4 μm, 1.5 μm, 2 μm, 3 μm, 5 μm, 10 μm, or more than 10 μm. Alternatively or additionally, sites of a first plurality of sites may have a pitch (e.g., an average pitch, minimum pitch, or maximum pitch) with respect to sites of a second plurality of sites of no more than about 10 μm, 5 μm, 3 μm, 2 μm, 1.5 μm, 1.4 μm, 1.3 μm, 1.2 μm, 1.1 μm, 1 μm, 900 nm, 800 nm, 700 nm, 600 nm, 500 nm, 400 nm, 300 nm, 250 nm, 200 nm, 100 nm, 50 nm, 10 nm, or less than 10 nm.

In some cases, an optically non-resolvable distance between a first array site and a second array site may be less than twice an emission wavelength of a signal (e.g., a photon) produced by a detectable label. In other cases, an optically non-resolvable distance may be less than twice an excitation wavelength of the first signal or less than half an excitation wavelength of a signal (e.g., a photon) produced by a detectable label.

In an aspect, provided herein is a method, comprising: a) providing a solid support containing a first site and a second site, in which the first site comprises a first analyte and a first immobilized avidity component, in which the second site comprises a second analyte and a second immobilized avidity component, in which the first immobilized avidity component differs from the second immobilized avidity component, and in which the first site is separated from the second site by an optically non-resolvable distance, b) coupling a first detectable probe to the first analyte at the first site, and coupling a second detectable probe to the second analyte at the second site, and c) detecting a first signal from the first detectable probe at the first site and detecting a second signal from the second detectable probe at the second site, in which the first detectable probe comprises: i) a first affinity agent that has a binding specificity for the first analyte, ii) a first mobile avidity component that has a binding specificity for the first immobilized avidity component, and iii) a first detectable label that is configured to produce the first signal, and wherein the second detectable probe comprises: i) a second affinity agent that has a binding specificity for the second analyte, ii) a second mobile avidity component that has a binding specificity for the second immobilized avidity component, and iii) a second detectable label that is configured to produce the second signal.

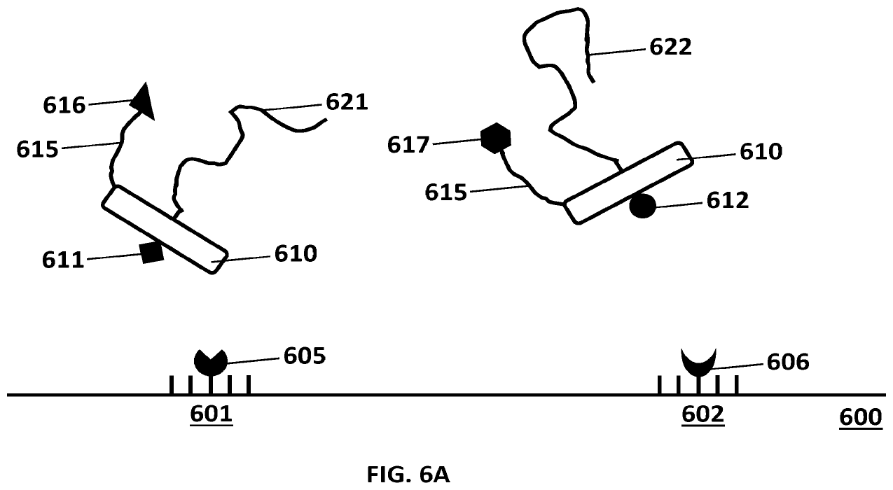

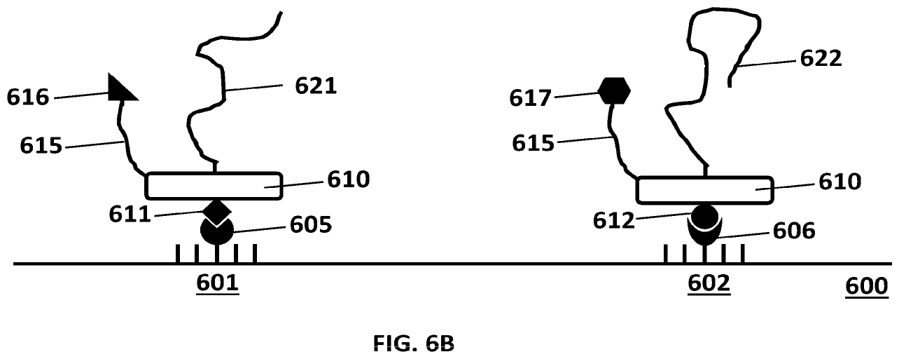

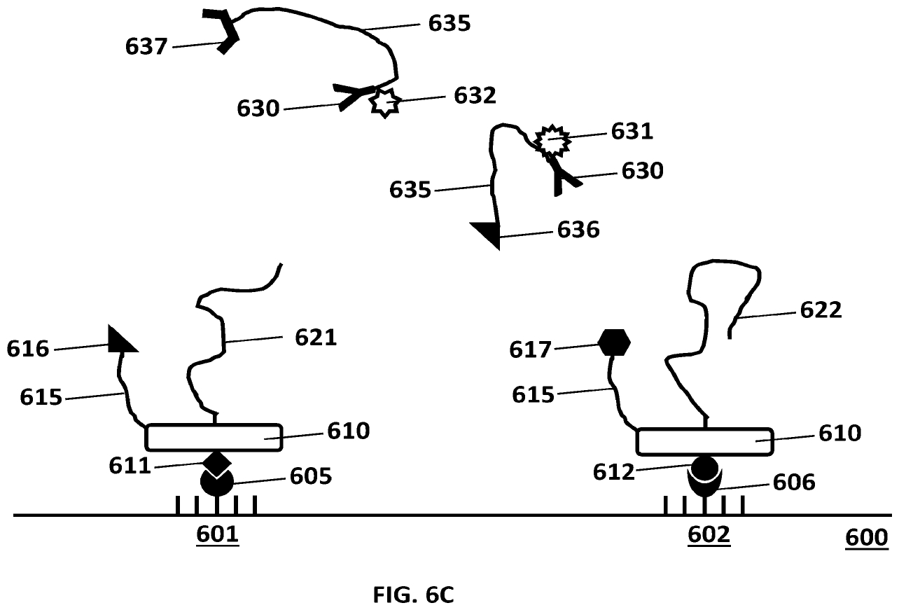

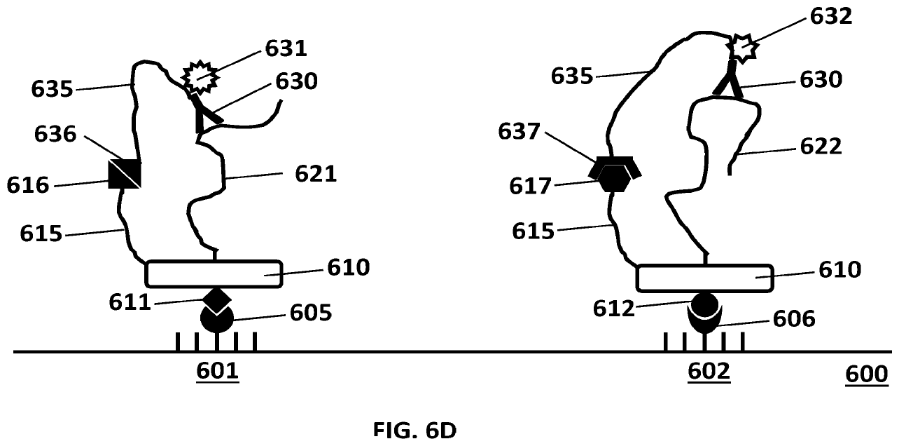

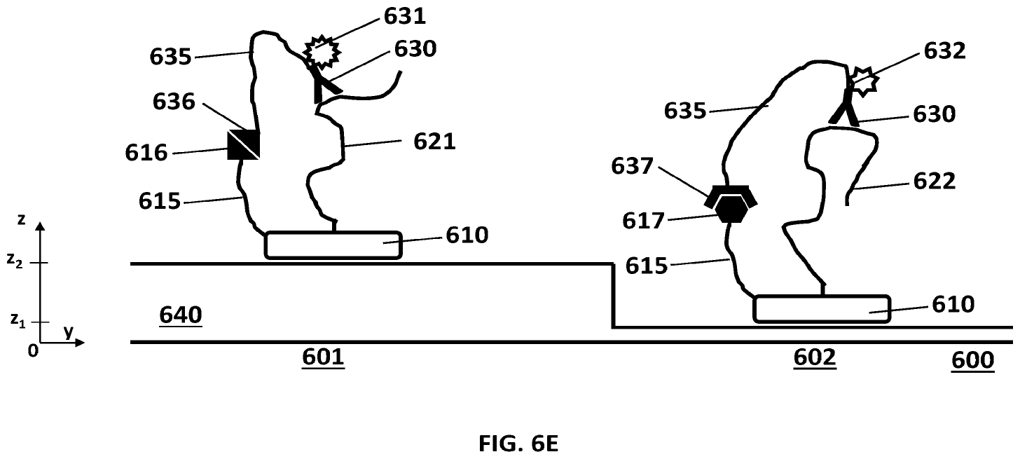

FIGS. 6A-6D depict a method of utilizing an array such as an array of FIGS. 5B-5C or FIGS. 7A-7C. The method may facilitate detection of analytes or detectable probes attached thereto on high-density analyte arrays when analyte-containing sites of such arrays are located at optically-non-resolvable addresses. FIG. 6A depicts a solid support 600 comprising array sites 601 and 602. Array sites 601 and 602 may be separated by an optically non-resolvable distance. Array site 601 comprises a first coupling moiety 605, and array site 602 does not comprise the first coupling moiety 605. Array site 602 comprises a second coupling moiety 606, and array site 601 does not comprise the second coupling moiety 606. Accordingly, array site 601 is configured to bind a moiety comprising a first complementary coupling moiety 611, and array site 602 is configured to bind a moiety comprising a second complementary coupling moiety 612. The array is contacted with a plurality of analytes, in which each individual analyte is contacted to a single anchoring group. A first analyte 621 is coupled to an anchoring group 610 that comprises a first complementary coupling group 611, and further comprises a first immobilized avidity component 616 that is attached to the anchoring group 610 by a linking moiety 615. A second analyte 622 is coupled to an anchoring group 610 that comprises a second complementary coupling group 612, and further comprises a second immobilized avidity component 617 that is attached to the anchoring group 610 by a linking moiety 615. As shown in FIG. 6B, the first analyte 621 is co-located with the first immobilized avidity component 616 at the first array site 601 by coupling of the first complementary coupling moiety 611 to the first coupling moiety 605. Likewise, the second analyte 622 is co-located with the second immobilized avidity component 617 at the second array site 602 by coupling of the second complementary coupling moiety 612 to the first coupling moiety 606.

Continuing with FIG. 6C, the solid support 600 is contacted with a plurality of detectable probes (e.g., contacting the solid support with a plurality of detectable probes in a fluidic medium). A first detectable probe comprises an affinity agent 630, a first detectable label 631, a first mobile avidity component 636, and an optional linking moiety 635 (e.g., a nanoparticle, a nucleic acid, a polymer, etc.) that binds together one or more components of the first detectable probe. A second detectable probe comprises an affinity agent 630, a second detectable label 632, a second mobile avidity component 637, and an optional linking moiety 635 (e.g., a nanoparticle, a nucleic acid, a polymer, etc.) that binds together one or more components of the first detectable probe. The affinity agent 630 may have a binding specificity for one or more epitopes that are present in analytes 621 and/or 622. Alternatively, the affinity agent 630 may not have a binding specificity for one or more epitopes that are present in analytes 621 and/or 622. The affinity agent that is attached to avidity component 636 may have different specificity compared to the affinity agent that is attached to avidity component 637. For example, the affinity agent that is attached to avidity component 636 can preferentially bind analyte 621 compared to analyte 622, and the affinity agent that is attached to avidity component 637 can preferentially bind analyte 622 compared to analyte 621. Accordingly, in some configurations, specificity of an affinity agent for a given site in an array can be driven by a combination of the affinity of its affinity agent component for an epitope at the site and the affinity of the mobile avidity component for an immobilized avidity component at the site.

As shown in FIG. 6D, the first detectable probe is bound to array site 601 by the binding of the affinity agent 630 to analyte 621 and the binding of the first mobile avidity component 636 to the first immobilized avidity component 616. Likewise, the second detectable probe is bound to array site 602 by the binding of the affinity agent 630 to analyte 622 and the binding of the second mobile avidity component 637 to the second immobilized avidity component 617. Subsequently, a signal from the first detectable label 631 may be detected on a first sensor at an address corresponding to array site 601, and a signal from the second detectable label 632 may be detected on a second sensor at an address corresponding to array site 602. Accordingly, array sites 601 and 602 may be optically resolvable by segregating different species of immobilized avidity components at specific sites or sets thereof.

FIG. 6E displays an alternative configuration of the array of FIG. 6D that may be advantageous for resolving array sites. A solid support 600 has a layered or deposited material 640, as set forth herein, disposed on a proximal surface of the solid support (i.e., z=0 of the provided z-axis). The layered or deposited material 640 is formed at varying thicknesses to provide array site 601 (with an outer surface at z=z₂) and array site 602 (with an outer surface at z=z₁). Analytes, anchoring groups, avidity components, and detectable probes may be located at sites 601 and 602 of FIG. 6E, as described for FIG. 6B-6D. The relative thicknesses of the layered or deposited material at array sites 601 and 602 may be selected such that a first signal from first detectable label 631 at array site 601 is amplified by constructive interference, and a second signal from second detectable label 632 at array site 602 is amplified by constructive interference.

It may be useful to provide an avidity component at an array site to facilitate controlled binding of detectable probes to analytes at the array site. An avidity component may comprise any suitable moiety or ligand that has one or more properties of: i) facilitating binding of a first detectable probe at the array site, in which the first detectable probe comprises a mobile avidity component that is configured to bind to an immobilized avidity component, ii) inhibiting binding of a second detectable probe at the array site, in which the second detectable probe does not comprise an avidity component that is configured to bind to the avidity component, and iii) facilitating retention of an affinity agent of the first detectable probe at the array site until the presence of the first detectable probe has been detected.

Table I presents pairs of complementary avidity components. An avidity component may be chosen from column A or B as an immobilized avidity component, and the complementary avidity component in the other column may be chosen as the mobile avidity component. An immobilized avidity component may be immobilized at an array site by covalent coupling to the array site (e.g., covalently coupled to a surface-coupled moiety of the array site), or by covalent coupling to an anchoring group or analyte attached to the array site. An immobilized avidity component may be immobilized at an array site by non-covalent coupling to the array site (e.g., non-covalently coupled to a surface-coupled moiety of the array site), or by non-covalent coupling to an anchoring group or analyte attached to the array site. In some cases, a non-covalently coupled immobilized avidity component may be configured to dissociate from an array site. For example, an immobilized avidity component may be dissociated from an array site by denaturation, change in pH, change in ionic strength, nucleic acid dehybridization, enzymatic cleavage, photocleavage, change in temperature, contact with a chemical denaturant, or any other suitable mechanism of disrupting the coupling of the immobilized avidity component to the array site. In some cases, after dissociating an immobilized a first avidity component from an array site, a second avidity component may be coupled to the array site.

TABLE I

| Avidity Component | Complementary Avidity Component |
|---|---|
| Oligonucleotide | Partially- or fully-complementary |
| | oligonucleotide |
| Oligonucleotide | Single-stranded DNA-binding protein |
| | (e.g., SSB, transcription promoters, |
| | transcription repressors) |
| Double-stranded nucleic | Double-stranded DNA-binding protein (e.g., |
| acid | histones) |
| Short peptide (e.g., less | Short peptide-specific affinity agent (e.g., |
| than about 100, 75, 50, 25, | antibody, aptamer, etc.) |
| 20, 15, 10, 5, 4, or 3 amino | |
| acids) | |
| Antibody | Antibody-binding protein (e.g., Protein A, |
| | Protein G, Protein L, Gamma Fc Receptor, |
| | etc.) |
| Enzymatic protein | Tethered enzymatic substrate (i.e., substrate |
| | covalently coupled to a linking moiety) |
| First complexing protein | Second complexing protein (e.g., second |
| (e.g., first procollagen) | procollagen) |
| Polyelectrolyte brush | Complementary protein molecule |
| molecule | |
| Chemical cross-linking | Complementary chemical cross-linking |
| agent | agent |
| Photo-catalyzed cross- | Complementary photo-catalyzed cross- |
| linking agent (e.g., thiol | linking agent |

A first array site may be distinguished from a second array site by the presence of a first immobilized avidity component at the first array site and a differing second immobilized avidity component at the second array site. Accordingly, a first detectable probe may be configured to bind to the first array site by comprising a complementary mobile avidity component to the first immobilized avidity component, and a second detectable probe may be configured to bind to the second array site by comprising a complementary mobile avidity component to the second immobilized avidity component. In some cases, a first immobilized avidity component may differ from a second immobilized avidity component with respect to type of avidity component (e.g., selected from different rows of Table I). For example, a first array site may comprise an immobilized polymer brush and a second array site may comprise an immobilized antibody-binding protein. In some cases, a first mobile avidity component may differ from a second mobile avidity component with respect to type of avidity component (e.g., selected from different rows of Table I). For example, a first detectable probe may comprise a protein that is bound by a polymer brush, and a second detectable probe may comprise an antibody that is bound by an antibody-binding protein. In some cases, a first immobilized avidity component and a second avidity component may be the same type of avidity component, but may differ with respect to a characteristic of the type of avidity component, such as a residue sequence (e.g., amino acid sequence, nucleotide sequence), a secondary or tertiary structure, a binding affinity, a binding specificity, or a combination thereof. For example, a first array site may comprise an immobilized oligonucleotide with a first nucleotide sequence and a second array site may comprise an immobilized oligonucleotide with a second nucleotide sequence.

Detectable probes comprising an affinity agent and a mobile avidity component may be designed to have an effective binding affinity, effective association rate (i.e., on-rate), and/or effective dissociation rate (i.e., off-rate). Selection of a suitable mobile avidity component to pair with a particular affinity agent will depend, at least in part, on the binding characteristics of the affinity agent. To inhibit unwanted detection events of a detectable probe (e.g., due solely to binding of the mobile avidity component to an immobilized avidity component in the absence of binding of the affinity agent to an analyte), it may be preferable to select a mobile avidity component with less binding affinity for its complementary immobilized avidity component relative to the binding affinity of the affinity agent for its analyte target. In some cases, it may be preferable to form a detectable probe comprising an affinity agent and a mobile avidity component, in which the association rate and dissociation rate of the avidity component with its binding partner are slower than the association rate and dissociation rate of the affinity agent with its binding partner (i.e., the mobile avidity component is slower to form a binding interaction and slower to dissociate from its binding interaction). In some cases, it may be preferable to form a detectable probe comprising an affinity agent and a mobile avidity component, in which the association rate and dissociation rate of the avidity component with its binding partner are faster than the association rate and dissociation rate of the affinity agent with its binding partner (i.e., the mobile avidity component is faster to form a binding interaction and faster to dissociate from its binding interaction).

For an array comprising two or more differing immobilized avidity components, a binding characteristic (e.g., binding affinity, association rate, dissociation rate) of a first immobilized avidity component may differ from (e.g., greater than, less than) a binding characteristic of a second immobilized avidity component. Likewise, for a plurality of detectable probes containing two or more mobile avidity components, a binding characteristic (e.g., binding affinity, association rate, dissociation rate) of a first immobilized avidity component may differ from (e.g., greater than, less than) a binding characteristic of a second immobilized avidity component. In some cases, a binding affinity of a first mobile avidity component for a first immobilized avidity component is weaker than a binding affinity of a first affinity agent for a first analyte. In some cases, a binding affinity of a second mobile avidity component for the second immobilized avidity component is weaker than a binding affinity of a second affinity agent for a second analyte. In some cases, a binding affinity of a first mobile avidity component for a first immobilized avidity component is stronger than a binding affinity of a second mobile avidity component for a first immobilized avidity component. In some cases, a binding affinity of a second mobile avidity component for a second immobilized avidity component is stronger than a binding affinity of a first mobile avidity component for a second immobilized avidity component.

Accordingly, a suitable avidity component may increase an effective binding on-rate for a detectable probe, decrease an effective binding off-rate of a detectable probe, or decrease an effective dissociation constant of a detectable probe. Without wishing to be bound by theory, an avidity component may facilitate retention of a bound detectable probe at an array site by increasing the overall strength of binding interactions that must be overcome to release the detectable probe from the array site.

An immobilized avidity component may be located at an array site. An immobilized avidity component may be covalently coupled to an array site. An immobilized avidity component may be non-covalently coupled to an array site. An immobilized avidity component may co-located with an analyte at an array site. An immobilized avidity component may be co-located with an analyte at an array site by a covalent coupling of the immobilized avidity component to the analyte. An immobilized avidity component may be co-located with an analyte at an array site by a non-covalent coupling of the immobilized avidity component to the analyte. An immobilized avidity component may be co-located with an analyte at an array site by a covalent coupling of the immobilized avidity component to an anchoring group that is coupled to the array site. An immobilized avidity component may be co-located with an analyte at an array site by a non-covalent coupling of the immobilized avidity component to an anchoring group that is coupled to the array site.

A method of may comprise coupling a first detectable probe to an analyte at a first array site and coupling a second detectable probe to an analyte at a second array site, in which the first array site is optically non-resolvable from the second array site. In some cases, coupling the first detectable probe to the first analyte and coupling the second detectable probe to the second analyte comprises simultaneously performing the steps of: i) coupling the first detectable probe to the first analyte, and ii) coupling the second detectable probe to the second analyte. For example, the first detectable probe and the second detectable probe may be simultaneously contacted to the array, thereby permitting simultaneous coupling of the probes. In other cases, coupling the first detectable probe to the first analyte and coupling the second detectable probe to the second analyte comprises sequentially performing the steps of: i) coupling the first detectable probe to the first analyte, and ii) coupling the second detectable probe to the second analyte. For example, coupling of a first detectable probe and a second detectable probe may be sequenced to include a detection of the first detectable probe before a coupling of the second detectable probe and subsequent detection of the second detectable probe.

A method of utilizing an array of analytes, as set forth herein, may comprise detecting a signal from a detectable probe at an array site. A detectable probe may comprise a detectable label such as a fluorophore or luminophore. Accordingly, a method of detecting a signal from a detectable probe comprising a detectable label may comprise stimulating the signal from the detectable label (e.g., providing a photon at an excitation wavelength of the detectable label, providing a thermal or chemical excitation source, etc.), thereby emitting the signal from the array site. After a signal from a detectable label of a detectable probe has been emitted, the signal may be detected on a detection device, for example by absorption of an emitted photon at a pixel of a pixel-based array.

In some cases, a method of utilizing an array of analytes, as set forth herein, may comprise detecting two or more differing signals from two or more differing detectable probes. For example, a detection method may be multiplexed by utilizing a first detectable probe and a second detectable probe, in which a first signal from the first detectable probe is distinguishable from a second signal from the second detectable probe (e.g., with respect to emission wavelength, with respect to fluorescence lifetime, etc.).

In some cases, detecting a first signal from a first detectable probe at a first array site and detecting a second signal from a second detectable probe at a second array site can comprise contacting the first array site and the second array site with a plurality of photons. In particular cases, a plurality of photons may comprise photons of a first excitation wavelength and photons of a second excitation wavelength, in which the first excitation wavelength is configured to produce the first signal from the first detectable label, and in which the second excitation wavelength is configured to produce the second signal from the second detectable label. In some cases, contacting the first array site and second array site with the plurality of photons comprises simultaneously contacting the first site with the photons of the first wavelength, and contacting the second site with photons of the second wavelength. In other cases, contacting the first site and second site with the plurality of photons comprises sequentially contacting the first site with photons of the first excitation wavelength, and contacting the second site with photons of the second excitation wavelength.

After a signal has been produced from a detectable probe at an array site, the signal may be detected by a detection device. The detection device can contain a sensor that is configured to receive the signal and assign a spatial address to where the signal originated. In some cases, a sensor may comprise a pixel-based array (e.g., a CCD pixel array, a CMOS pixel array). At a given instant during emission and detection of a signal, a pixel-based array may be aligned with an array site such that one or more pixels of the array correspond to the spatial location of the array site. In some cases, a signal emitted from an array site may be sensed by one or more pixels, such as at least about 1, 2, 3, 4, 5, 6, 7, 8, 9, 10, 15, 16, 20, 25, 30, 35, 36, 40, 45, 49, 50, 60, 64, 70, 80, 81, 90, 100, or more than 100 pixels of a pixel-based array. Alternatively or additionally, a signal emitted from an array site may be sensed by no more than about 100, 90, 81, 80, 70, 64, 60, 50, 49, 45, 40, 36, 35, 30, 25, 20, 16, 15, 10, 9, 8, 7, 6, 5, 4, 3, 2, or less than 2 pixels.

A pixel of a pixel-based array may be configured to receive a signal of a particular wavelength or a range of wavelengths. In some cases, a sensor may further comprise a filter that only transmits a wavelength or range of wavelengths to a pixel of a sensor. A pixel may receive a photon of an emission wavelength of at least about 200 nanometers (nm), 220 nm, 240 nm, 260 nm, 280 nm, 300 nm, 320 nm, 340 nm, 360 nm, 380 nm, 400 nm, 420 nm, 440 nm, 460 nm, 480 nm, 500 nm, 520 nm, 540 nm, 560 nm, 580 nm, 600 nm, 620 nm, 640 nm, 660 nm, 680 nm, 700 nm, 720 nm, 740 nm, 760 nm, 780 nm, 800 nm, 820 nm, 840 nm, 860 nm, 880 nm, 900 nm, 920 nm, 940 nm, 960 nm, 980 nm, 1000 nm, or more than 1000 nm. Alternative or additionally, a pixel may receive a photon of no more than about 1000 nm, 980 nm, 960 nm, 940 nm, 920 nm, 900 nm, 880 nm, 860 nm, 840 nm, 820 nm, 800 nm, 780 nm, 760 nm, 740 nm, 720 nm, 700 nm, 680 nm, 660 nm, 640 nm, 620 nm, 600 nm, 580 nm, 560 nm, 540 nm, 520 nm, 500 nm, 480 nm, 460 nm, 440 nm, 420 nm, 400 nm, 380 nm, 360 nm, 340 nm, 320 nm, 300 nm, 280 nm, 260 nm, 240 nm, 220 nm, 200 nm, or less than 200 nm.

In some cases, such as multiplexed detection of detectable probe binding (i.e., detection of two or more types of distinguishable detectable probes), detection may be performed on a detection device containing two or more sensors. For example, a detection device may comprise a first sensor (e.g., a sensor containing a first pixel-based array) that is configured to receive a first signal from a first detectable probe, and may further comprise a second sensor (e.g., a sensor containing a second pixel-based array) that is configured to receive a second signal from a second detectable probe. Methods for spatially separating distinguishable signals (e.g., use of dichroic mirrors) are known in the art. In some cases, detecting a first signal from a first detectable probe at a first array site and detecting a second signal from a second detectable probe at a second array site can further comprise detecting the first signal on a first pixel-based sensor, and detecting the second signal on a second pixel-based sensor. In some cases, a first pixel-based sensor may be disposed on a first solid support and a second pixel-based sensor may be disposed on a second solid support. In other cases, a first pixel-based sensor and a second pixel-based sensor may be disposed on a single solid support. In other cases, it may be possible to use a single pixel-based sensor to two or more signals. For example, a detection method may utilize a first scan of array site utilizing a first chromatic filter that transmits a first wavelength of light or range thereof, and a second scan of the array sites using a second chromatic filter that transmits a second wavelength of light or range thereof. In some cases, detecting a first signal from a first detectable probe at a first site and detecting a second signal from a second detectable probe at a second site can further comprise detecting the first signal at a first pixel of a pixel-based sensor and detecting the second signal at a second pixel of the pixel-based sensor.

A method of utilizing an array, as set forth herein, may comprise a step of dissociating a detectable probe from an array site. In some cases, dissociating a detectable probe from an array site may comprise contacting the array site with a probe dissociation medium that is configured to disrupt a binding interaction between a detectable probe and an analyte and/or immobilized avidity component to which the detectable probe is bound. A probe dissociation medium may comprise a fluidic medium, and may further comprise a dissociation agent such as a denaturant, a chaotrope, or a surfactant (e.g., an anionic surfactant, a cationic surfactant, a zwitterionic surfactant, a non-ionic surfactant). Contacting an array site with a probe dissociation medium may further comprise providing a change of a fluidic property such as ionic strength, polarity, pH, or temperature.

A method of utilizing an array, as set forth herein, may further comprise repeating one or more steps (e.g., contacting a detectable probe to an array site, binding the detectable probe to an analyte at the array site, detecting the detectable probe at the array site, dissociating the detectable probe from analyte at the array site, etc.). A method may comprise a cyclical method, in which one or more steps are repeated serially. In some cases, a plurality of cycles of a cyclical method may comprise contacting a differing detectable probe to an array. For example, a different detectable probe may be provided during each cycle of a plurality of cycles, in which each differing detectable probe is distinguished by a differing binding specificity. In some cases, a plurality of cycles of a cyclical method may comprise contacting the same detectable probe to an array. For example, a detectable probe may be provided during each cycle of a plurality of cycles, in which the detectable probe has a same binding specificity.

For a multiplexed assay, a method may comprise a sequence or steps of a cycle of steps, in which two or more distinguishable detectable probes are provided for each detectable probe contacting step. In some cases, a method may further comprise: d) dissociating a first detectable probe from a first array site and dissociating a second detectable probe from a second array site. In some cases, a method may further comprise: e) contacting a third detectable probe to the solid support, in which the third detectable probe comprises: i) a third affinity agent, ii) the first mobile avidity component that has a binding specificity for the first immobilized avidity component, and iii) the first detectable label that is configured to produce the first signal. In some cases, the third affinity agent may comprise a binding specificity for the first analyte. Accordingly, a method may further comprise a step of: f) detecting the first signal from the third detectable probe at the first site. In other cases, the third affinity agent may not comprise a binding specificity for the first analyte. Accordingly, a method may further comprise a step of: f) detecting an absence of the first signal from the third detectable probe at the first site.

In another aspect, provided herein is a method, comprising: a) providing a solid support comprising a first plurality of sites and a second plurality of sites, in which each site of the first plurality of sites has a first elevation, in which each site of the second plurality of sites has a second elevation, in which a plurality of analytes is coupled to the first plurality of sites and the second plurality of sites, in which a site of the first plurality of sites is an optically non-resolvable distance from a site of the second plurality of sites, and in which detectable probes are coupled to analytes of the plurality of analytes, b) detecting on a detection device a first set of signals from detectable probes, in which the detection device has a first focal plane that corresponds to the first elevation, c) detecting on the detection device a second set of signals from detectable probes, in which the detection device has a second focal plane that corresponds to the second elevation, and d) based upon the first set of signals and the second set of signals, determining a first set of sites of the first plurality of sites containing detectable probes, and determining a second set of sites of the second plurality of sites containing detectable probes.

In another aspect, provided herein is a method comprising: a) providing a solid support comprising a first plurality of sites and a second plurality of sites, in which each site of the first plurality of sites has a first elevation, in which each site of the second plurality of sites has a second elevation, in which a plurality of analytes is coupled to the first plurality of sites and the second plurality of sites, in which a site of the first plurality of sites is an optically non-resolvable distance from a site of the second plurality of sites, and in which detectable probes are coupled to analytes of the plurality of analytes, b) detecting on a detection device signals from detectable probes, in which the detection device comprises a single sensor, in which the single sensor comprises a first pixel array and a second pixel array, in which the first pixel array is spatially separated from the second pixel array on the single sensor, in which the first pixel array is oriented at a focal point of signals from detectable probes at the first plurality of sites, and in which the second pixel array is oriented at a focal point of signals from detectable probes at the second plurality of sites, and c) based upon the first set of signals and the second set of signals, determining a first set of sites of the first plurality of sites containing detectable probes, and determining a second set of sites of the second plurality of sites containing detectable probes.

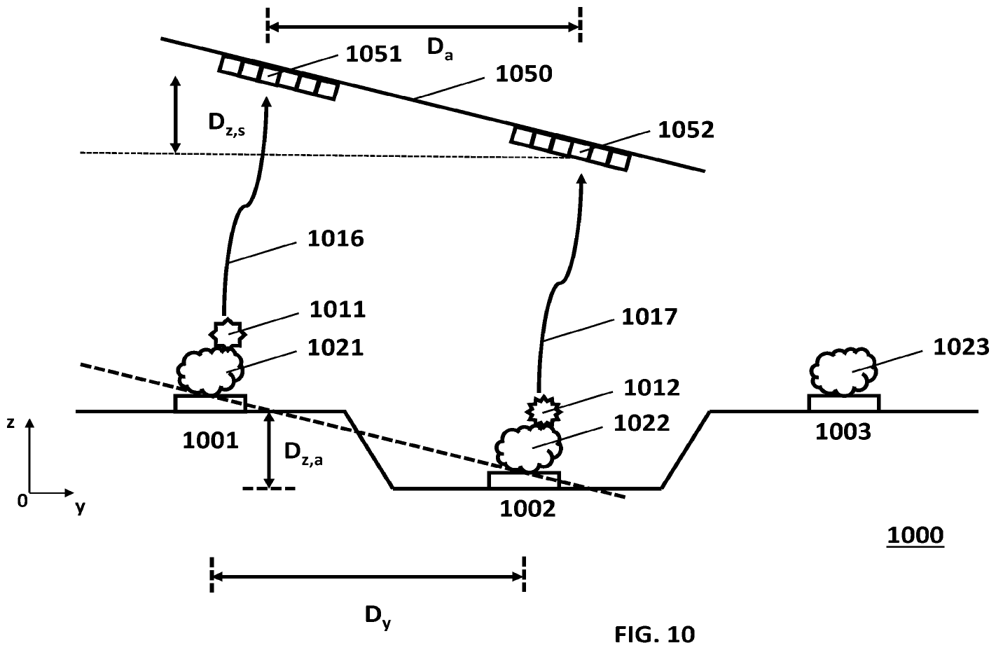

FIG. 10 depicts a configuration of a detection system for detecting two differing detectable probes on a single sensor containing multiple detection channels. A solid support 1000 contains array sites 1001, 1002, and 1003. Array sites 1001 and 1003 are disposed on non-contiguous surfaces that have a substantially same z-axis height. Array site 1002 is disposed on a surface within a channel or depression. Accordingly a z-axis height of array site 1002 differs from a z-axis height of array sites 1001 and 1003 by a height of D_{z,a}. Adjacent arrays sites 1001 and 1002 are separated by a centerpoint-to-centerpoint distance of D_{y}. The distance D_{y} is optionally an optically non-resolvable distance. Arrays sites 1001, 1002, and 1003 contains analytes 1021, 1022, and 1023, respectively. Detectable probes 1011 and 1012 are bound to analytes 1021 and 1022, respectively. Detectable probe 1011 provides a first signal 1016 at array site 1001 (e.g., a photon of a first emission wavelength) and detectable probe 1012 provides a second signal 1017 at array site 1002 (e.g., a photon of a second emission wavelength). The first signal 1016 and the second signal 1017 are transmitted to a sensor of a detection device (e.g., a microscope, a camera). The sensor comprises a single solid support 1050 containing a first set of pixels 1051 that is configured to detect the first signal 1016, and a second set of pixels 1052 that is configured to detect the second signal 1017. The first set of pixels 1051 is spatially separated from the second set of pixels 1052 on the solid support 1050 by a distance Dₐ, which is optionally different from distance D_{y}. The sensor is oriented at an angle relative to the substantially horizontal surfaces of substrate 1000. A chosen angle of orientation of the sensor may be selected to obtain maximal signals from arrays sites 1001 and 1002, respectively. In some cases, the chosen angle of orientation of the sensor may be chosen such that the z-axis offset distance D_{z,s} between the first set of pixels 1051 and the second set of pixels 1052 is substantially the same as D_{z,a}. Such a configuration may be useful if a difference in z-axis heights between array sites produces a difference in optimum focal planes for respective signals emitted from the array sites. In some cases, the chosen angle of orientation of the sensor may be chosen such that the z-axis offset distance D_{z,s} between the first set of pixels 1051 and the second set of pixels 1052 is greater than or less than D_{z,a}. Such a configuration may be chosen if, for example, a layered or deposited material is provided on the solid support 1000, thereby producing unique optimum focal planes based upon signal wavelength and layer thickness. In some cases, a sensor may be oriented substantially horizontal to a surface of a solid support comprising array sites. FIG. 10 does not depict additional elements of a detection device that may affect the collection and focusing of light emitted from array sites. A chosen angle of orientation of a sensor may be chosen with respect to an optical element, such as a light-collecting element (e.g., an objective lens, an aperture, etc.). Additional aspects of single-analyte detection with a single sensor containing multiple channels are described in U.S. patent application Ser. No. 18/180,733, which is hereby incorporated by reference in its entirety.

Also provided herein are systems for performing a method, as set forth herein. In an aspect, provided herein is a system, comprising: a) a solid support comprising a first plurality of sites and a second plurality of sites, wherein each individual site of the first plurality of sites is an optically resolvable distance from each other site of the first plurality of sites, wherein each individual site of the second plurality of sites is an optically resolvable distance from each other site of the second plurality of sites, b) a plurality of sample analytes coupled to sites of the first plurality of sites and sites of the second plurality of sites, wherein a first site of the first plurality of sites comprises a first sample analyte of the plurality of analytes, wherein a second site of the second plurality of sites comprises a second sample analyte of the plurality of analytes, and wherein the first site and the second site are an optically non-resolvable distance apart, c) a first detectable probe bound to the first sample analyte at the first site, and a second detectable probe bound to the second sample analyte at the second site, and d) a detection device comprising a first pixel-based array and a second pixel-based array, wherein a first signal from the first detectable probe contacts the first pixel-based array, and wherein a second signal from the second detectable probe contacts the second pixel-based array.

Methods and systems set forth herein may utilize arrays of analytes. In some cases, an array of analytes may comprise a first array site and a second array site, in which the first array site and the second array site are optically non-resolvable. In other cases, an array of analytes may comprise a first array site and a second array site, in which the first array site and the second array site are optically resolvable. In some cases, an array of analytes may comprise a first plurality of array sites and a second plurality of array sites, in which one or more array sites of the first plurality of array sites are optically non-resolvable from one or more array sites of the second plurality of array sites. In some cases, an array of analytes may comprise a plurality of array sites, in which each individual array site is optically resolvable from at least one other array site of the plurality of array sites.

An array of analytes provided to a method or system, as set forth herein, may be distinguished by characteristics of the analytes distributed thereupon. A plurality of analytes provided on an array of analytes may be heterogeneous with respect to one or more characteristics (e.g., analyte species, analyte isoform, analyte state, dynamic range, etc.). For example, a plurality of polypeptides may comprise two or more species of polypeptides. In a particular case, a first species of the two or more species of polypeptides may have a characterizable or known dynamic range with respect to a second species of the two or more species of polypeptides. In another example, a plurality of polypeptides may comprise two or more proteoforms of a species of polypeptide (e.g., splice variants, post-translational modification variants, etc.). In another example, a plurality of polypeptides may comprise two or more states of a polypeptide species (e.g., pre-modification, post-translationally modified, partially degraded, complexed with a second polypeptide, etc.). Additionally or alternatively, a plurality of analytes may be provided on an array of analytes from a sample source (e.g., a biological organism, a non-biological organism), in which the plurality of analytes contains a measure of population diversity with respect to the sample source. For example, a plurality of analytes may contain a characterizable or known fraction of analyte species diversity for a proteome, genome, or transcriptome. In another example, a plurality of analytes may contain a characterizable or known fraction of analyte species diversity for a microbiome.

In some cases, an array of analytes may be multiplexed. A multiplexed array of analytes may comprise a first plurality of analytes coupled to a first set of array sites of a plurality of array sites, and a second plurality of analytes coupled to a second set of array sites of the plurality of array sites. In some cases, a multiplexed array of analytes may comprise a first plurality of analytes coupled to a first set of array sites of a plurality of array sites, and a second plurality of analytes coupled to a second set of array sites of the plurality of array sites, in which the first set of array sites and the second set of array sites have a random spatial distribution. A multiplexed array of analytes may comprise two or more pluralities of analytes, in which a first plurality of analytes and a second plurality of analytes differ with respect to a characterizable or known degree of heterogeneity or population diversity. A multiplexed array of analytes may comprise a first plurality of sample analytes and a second plurality of sample analytes. A multiplexed array of analytes may comprise a first plurality of analytes and a second plurality of analytes, in which the first plurality of analytes and the second plurality of analytes are obtained from the same sample source (e.g., same biological organism, same biological or non-biological system), and optionally in which the first plurality of analytes and the second plurality of analytes differ from each other with respect to a characterizable or known measure of heterogeneity or population diversity. In some cases, a multiplexed array of analytes may comprise a first plurality of analytes and a second plurality of analytes, in which the first plurality of analytes and the second plurality of analytes are obtained from different sample sources, respectively. For example, the sample sources can include samples from different individuals of the same organism type (e.g., samples from different humans), samples that have been treated differently (e.g., a sample treated with a therapeutic agent and a control sample not treated with the agent), or samples from different organisms.

Accordingly, an array of analytes formed with a plurality of analytes containing a characterizable or known degree of heterogeneity or population diversity may contain unique spatial arrangements of analytes. In some cases, an array of analytes can comprise a plurality of analytes with a measure of heterogeneity or diversity, as set forth herein, in which the analytes comprise a random spatial distribution (i.e., the address of any analyte of the plurality of analytes cannot be predicted based upon a priori information). Accordingly, an array of analytes may comprise a first array site comprising a first analyte and a second array site comprising a second analyte, in which the first analyte and the second analyte differ (e.g., with respect to analyte species, analyte isoform, analyte state, dynamic range, etc.). In some cases, the array site containing the first analyte and the array site containing the second analyte may be optically non-resolvable.

An array of analytes may comprise a plurality of analytes, in which the plurality of analytes comprises at least about 2, 5, 10, 25, 50, 100, 250, 500, 1000, 2500, 5000, 10000, 15000, 20000, 25000, 30000, 50000, 100000, 500000, 1000000, or more than 1000000 species of analytes. Alternatively or additionally, a plurality of analytes may comprise no more than about 1000000, 500000, 100000, 50000, 30000, 25000, 20000, 15000, 10000, 5000, 2500, 1000, 500, 250, 100, 50, 25, 10, 5, 2, or less than 2 species of analytes.

An array of analytes may comprise a plurality of analytes, in which the plurality of analytes comprises at least about 0.0001%, 0.001%, 0.01%, 0.1%, 0.5%, 1%, 5%, 10%, 15%, 20%, 25%, 30%, 35%, 40%, 45%, 50%, 55%, 60%, 65%, 70%, 75%, 80%, 85%, 90%, 95%, 99%, 99.5%, 99.9%, 99.99%, 99.999%, 99.9999%, or more than 99.9999% of the analyte species diversity of a proteome, genome, transcriptome, or metabolome. Alternatively or additionally, a plurality of analytes may comprise no more than about 99.9999%, 99.999%, 99.99%, 99.9%, 99.5%, 99%, 95%, 90%, 85%, 80%, 75%, 70%, 65%, 60%, 55%, 50%, 45%, 40%, 35%, 30%, 25%, 20%, 15%, 10%, 5%, 1%, 0.5%, 0.1%, 0.01%, 0.001%, 0.0001%, or less than 0.0001% of the analyte species diversity of a proteome, genome, transcriptome, or metabolome.

An array of analytes may comprise a plurality of analytes, in which at least about 1, 2, 3, 4, 5, 10, 15, 20, 25, 30, 40, 50, 75, 100, 150, 200, 300, 400, 500, 1000, or more than 1000 isoforms of a species of an analyte of the plurality of analytes are present on the array. Alternatively or additionally, an array of analytes may comprise a plurality of analytes, in which no more than about 1000, 500, 400, 300, 200, 150, 100, 75, 50, 40, 30, 25, 20, 15, 10, 5, 4, 3, 2, or less than 2 isoforms of a species of an analyte of the plurality of analytes are present on the array.

An array of analytes may comprise a plurality of analytes, in which at least about 1%, 5%, 10%, 15%, 20%, 25%, 30%, 35%, 40%, 45%, 50%, 55%, 60%, 75%, 80%, 85%, 90%, 95%, 99%, 99.9%, or more than 99.9% of isoform diversity of a species of an analyte of the plurality of analytes is present on the array. Alternatively or additionally, an array of analytes may comprise a plurality of analytes, in which no more than about 99.9%, 99%, 95%, 90%, 85%, 80%, 75%, 70%, 65%, 60%, 55%, 50%, 45%, 40%, 35%, 30%, 25%, 20%, 15%, 10%, 5%, 1%, or less than 1% of isoform diversity of a species of an analyte of the plurality of analytes are present on the array.

An array of analytes may comprise a first analyte at a first array site and a second analyte at a second array site, in which the first analyte comprises a first species of analyte, in which the second analyte comprises a second species of analyte, and in which the dynamic range of the first species of analyte relative to the second species of analyte in the plurality of analytes is at least about 10, 10², 10³, 10⁴, 10⁵, 10⁶, 10⁷, 10⁸, 10⁹, 10¹⁰, 10¹¹, 10¹², or more than 10¹². Alternatively or additionally, the dynamic range of the first species of analyte relative to the second species of analyte in the plurality of analytes is no more than about 10¹², 10¹¹, 10¹⁰, 10⁹, 10⁸, 10⁷, 10⁶, 10⁵, 10⁴, 10³, 10², 10 or less than 10.

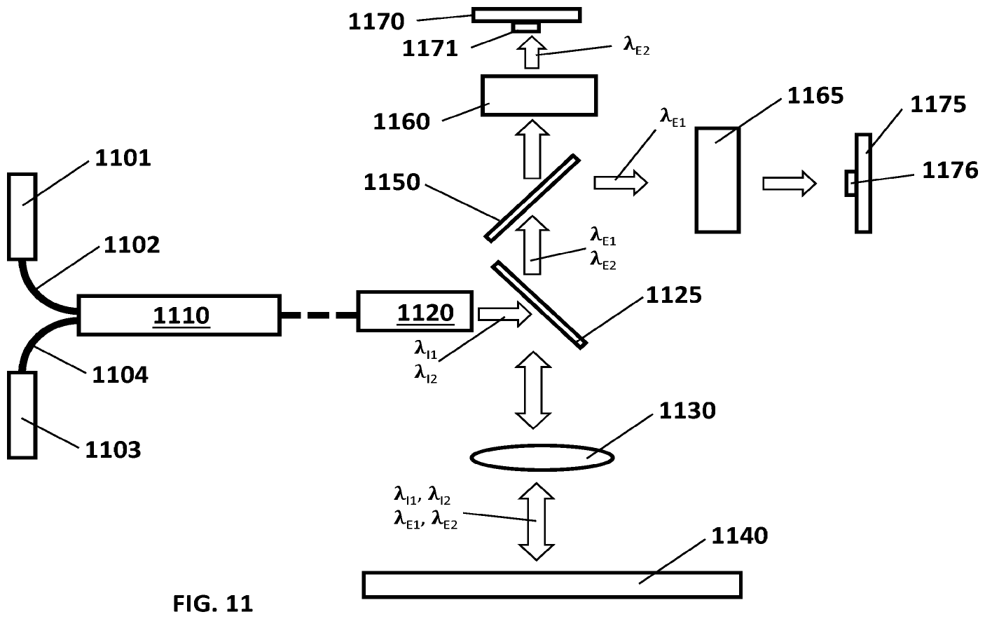

Further set forth herein are systems for performing methods, as set forth herein. FIG. 11 illustrates a possible configuration of an optical detection system that may be useful for detecting a presence or absence of signal from a detectable probe at an array site of an array. The system depicted in FIG. 11 contains an illumination pathway with an epi-illumination configuration. A first light source 1101 and a second light source 1103 (e.g., lamp, laser, light bulb, filament, light-emitting diode, etc.) are optically connected to beam-shaping optics 1110 (e.g., filters, polarizing lenses, collimating lenses, beam splitters, etc.) by optional waveguides (e.g., fiberoptic cables, etc.) 1102 and 1104, respectively. In a first optional configuration, a first illumination beam containing light of wavelength λ_{I1} from the first light source 1101 and a spatially-separated second illumination beam containing light of wavelength λ_{I2} from the second light source 1103 are transmitted from the beam-combining optics 1110, for example by a prismatic beam-splitter. In a second optional configuration, a combined light beam containing light from the first light source 1101 and the second light source 1103 is formed in a beam-combining optical element 1110, and optionally passed through additional beam-shaping optical elements 1120. Light from the first light source 1101 and the second light source 1103 is directed to an illumination target 1140 (e.g., a solid support) by contacting an optional mirror 1125 (e.g., a dichroic mirror, etc.) and passing through an objective lens 1130. After light from the first light source 1101 and the second light source 1103 contact the illumination target 1140, light from a first signal source and light from a second signal source pass through the objective lens 1130 and optionally pass through the mirror 1125 before entering the branched portion of the emission pathway. Light of wavelength λ_{E2} from the second signal source passes through a beam-splitting element 1150 (e.g., a dichroic mirror, a beam splitter, etc.) and optional beam-shaping optics 1160 before contacting a second sensor 1170 at a second channel 1171 that is configured to detect light from the second signal source. Light of wavelength λ_{E1} from the first signal source is redirected by the beam-splitting element 1150 and passes through optional beam-shaping optics 1165 before contacting a first sensor 1175 at a first channel 1176 that is configured to detect light from the first signal source.

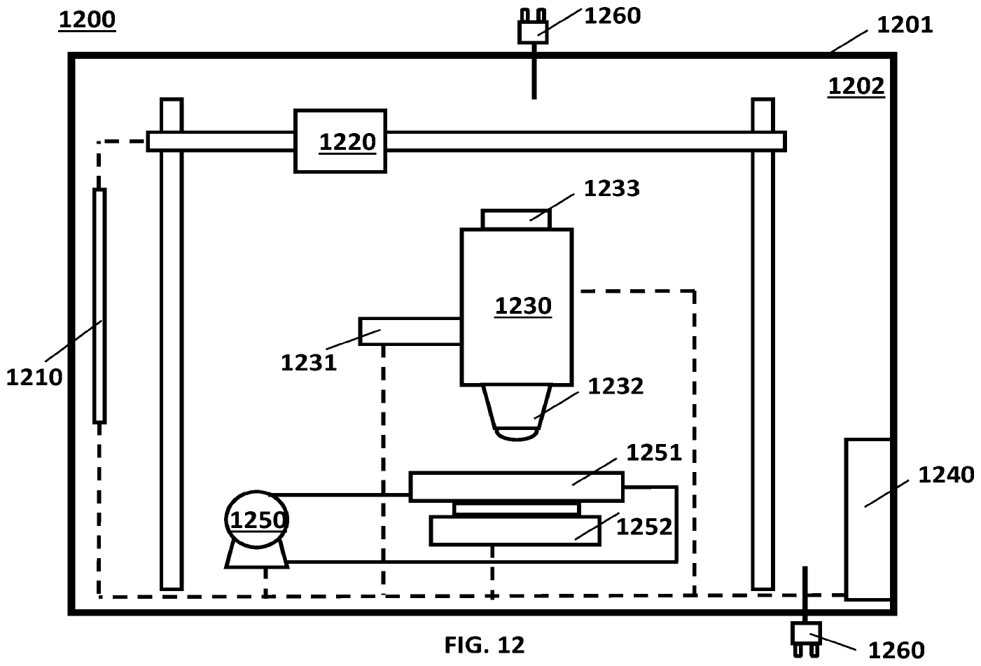

Additional components of an array-based system are shown in FIG. 12. FIG. 12 illustrates an operational system 1200 comprising a plurality of components that are enclosed in a space 1202 that is surrounded by a housing 1201. The plurality of components may comprise a processor or microprocessor 1210 that implements a processor-based operations (e.g., running control operations, performing calculations, etc.). The processor or microprocessor 1210 may be in communication with (as indicated by dashed lines), and/or in control of, one or more additional components, including a robotic apparatus 1220 (e.g., an automated injector, a sample-handling system, etc.), an optical detection system 1230 comprising a light source 1231, an objective lens 1232, and a single-channel or multi-channel sensor 1233, a thermal control device 1240 (e.g., a fan, a heat exchanger, etc.), and a fluidics system comprising a pump 1250, a fluidic cartridge 1251, and a motion controller 1252. Operational system temperatures or temperature profiles may be measured by one or more thermocouples 1260. In some cases, a component of an optical detection system (e.g., a sensor, an optical device, etc.) may comprise a thermoelectric cooling system that is configured to maintain the component at a temperature above or below a threshold temperature for operation.

In some cases, a system, such as the system depicted in FIG. 12, may comprise a processor that is configured to receive signal information from a detection device (e.g., a device comprising a sensor). In some cases, a processor may receive a first set of signal information from a first pixel-based array, and may further receive a second set of signal information from a second pixel-based array. In some cases, a first set of signal information can comprise a first image containing a first signal from a first detectable probe, and a second set of signal information can comprise a second image containing a second signal from a second detectable probe. In some cases, a system may further comprise a computer-readable storage medium. The computer-readable storage medium may be configured to send and receive information from a processor. In some cases, a processor or a computer-readable storage medium may comprise an image analysis process. In some cases, the computer-readable storage medium can further comprise a data structure containing a spatial address of the first site on a solid support, a spatial address of the second site on a solid support, a classification of the first signal at a first array site, and a classification of a second signal at a second array site. A classification of a signal from a detectable probe at an array site may comprise a quantitative classification, such as a signal intensity or a signal lifetime. A classification of a signal from a detectable probe at an array site may comprise a qualitative classification, such as (PRESENT/NOT PRESENT/UNCERTAIN). In some cases, a computer-readable storage medium can further comprise a data structure containing a spatial address of each array site of a plurality of array sites on a solid support, and a classification of a signal at each array site of the plurality of array sites.

Structured arrays provided herein may be combined with various detection devices to form single-analyte detection systems. FIGS. 11 and 12 depict systems that may be utilized for confocal laser scanning microscopy, but other techniques such as any suitable form of super-resolution microscopy may be utilized to detect signals on arrays set forth herein. For example, useful system may include systems configured to perform structured illumination microscopy, stimulated emission depletion (STED) microscopy, stochastic optical reconstruction microscopy (STORM), photoactivated localization microscopy (PALM), and fluorescent photoactivated localization microscopy (fPALM).

Structured illumination microscopy may utilize a spatially modulated light source to extract additional signal information (e.g., phase information) from an image generated by an optical device. The additional information can facilitate higher resolution reconstruction of an object at an array site. In some cases, structured illumination microscopy may be utilized to provide phase information from a reflecting plane rather than using a diffraction grating. In some cases, an optical technique used to generate structured illumination can also be utilized to detect signals at differing sets of array sites in different detection cycles (e.g., imaging sites containing a first avidity component in a first detection cycle, then imaging sites containing a second avidity component in a second detection cycle).

In some cases, stimulated emission depletion (STED) or related techniques such as STORM, PALM, or fPALM, may be utilized to provide timed pulses of light to spatially modulate active fluorophores at array sites. Arrays provided herein may be especially useful for STED due to the spatial separation of analytes, thereby facilitating precise activation or de-activation of detectable labels or other photoactive moieties at array sites.

In some cases, a super-resolution microscopy technique may be utilized to provide a series of images of an array or a region thereof, thereby facilitating reconstruction of a more detailed final image of the array or the region thereof. In some cases, an optical system may further comprise an autofocus device. An autofocus device may be advantageous for adjusting focus between differing focal planes that correspond to differing array surface elevations with respect to an optical axis of the optical system.

### Polypeptide Assays

The present disclosure provides compositions, apparatus and methods that can be useful for characterizing sample components, such as proteins, nucleic acids, cells, or other species, by obtaining multiple separate and non-identical measurements of the sample components. In particular configurations, the individual measurements may not, by themselves, be sufficiently accurate or specific to make the characterization, but an aggregation of the multiple non-identical measurements can allow the characterization to be made with a high degree of accuracy, specificity, and confidence. For example, the multiple separate measurements can include subjecting the sample to reagents that are promiscuous with regard to recognizing multiple components of the sample. Accordingly, a first measurement carried out using a first promiscuous reagent may perceive a first subset of sample components without distinguishing one component from another. A second measurement carried out using a second promiscuous reagent may perceive a second subset of sample components, again, without distinguishing one component from another. However, a comparison of the first and second measurements can distinguish: (i) a sample component that is uniquely present in the first subset but not the second; (ii) a sample component that is uniquely present in the second subset but not the first; (iii) a sample component that is uniquely present in both the first and second subsets; or (iv) a sample component that is uniquely absent in the first and second subsets. The number of promiscuous reagents used, the number of separate measurements acquired, and degree of reagent promiscuity (e.g., the diversity of components recognized by the reagent) can be adjusted to suit the component diversity expected for a particular sample.

The present disclosure provides assays that are useful for detecting one or more analytes. Exemplary assays are set forth herein in the context of detecting proteins. Those skilled in the art will recognize that methods, compositions, and apparatus set forth herein can be adapted for use with other analytes such as nucleic acids, polysaccharides, metabolites, vitamins, hormones, enzyme co-factors and others set forth herein or known in the art. Particular configurations of the methods, apparatus and compositions set forth herein can be made and used, for example, as set forth in U.S. Pat. No. 10,473,654 or US Pat. App. Pub. Nos. 2020/0318101 A1 or 2020/0286584 A1, each of which is incorporated herein by reference. Exemplary methods, systems and compositions are set forth in further detail below.

A composition, apparatus or method set forth herein can be used to characterize an analyte, or moiety thereof, with respect to any of a variety of characteristics or features including, for example, presence, absence, quantity (e.g., amount or concentration), chemical reactivity, molecular structure, structural integrity (e.g., full length or fragmented), maturation state (e.g., presence or absence of pre- or pro-sequence in a protein), location (e.g., in an analytical system, subcellular compartment, cell or natural environment), association with another analyte or moiety, binding affinity for another analyte or moiety, biological activity, chemical activity or the like. An analyte can be characterized with regard to a relatively generic characteristic such as the presence or absence of a common structural feature (e.g., amino acid sequence length, overall charge or overall pKa for a protein) or common moiety (e.g., a short primary sequence motif or post-translational modification for a protein). An analyte can be characterized with regard to a relatively specific characteristic such as a unique amino acid sequence (e.g., for the full length of the protein or a motif), an RNA or DNA sequence that encodes a protein (e.g., for the full length of the protein or a motif), or an enzymatic or other activity that identifies a protein. A characterization can be sufficiently specific to identify an analyte, for example, at a level that is considered adequate or unambiguous by those skilled in the art.

In particular configurations, a protein can be detected using one or more affinity agents having known or measurable binding affinity for the protein. For example, an affinity agent can bind a protein to form a complex and a signal produced by the complex can be detected. A protein that is detected by binding to a known affinity agent can be identified based on the known or predicted binding characteristics of the affinity agent. For example, an affinity agent that is known to selectively bind a candidate protein suspected of being in a sample, without substantially binding to other proteins in the sample, can be used to identify the candidate protein in the sample merely by observing the binding event. This one-to-one correlation of affinity agent to candidate protein can be used for identification of one or more proteins. However, as the protein complexity (i.e. the number and variety of different proteins) in a sample increases, or as the number of different candidate proteins to be identified increases, the time and resources to produce a commensurate variety of affinity agents having one-to-one specificity for the proteins approaches limits of practicality.

Methods set forth herein, can be advantageously employed to overcome these constraints. In particular configurations, the methods can be used to identify a number of different candidate proteins that exceeds the number of affinity agents used. For example, the number of candidate proteins identified can be at least 5×, 10×, 25×, 50×, 100× or more than the number of affinity agents used. This can be achieved, for example, by (1) using promiscuous affinity agents that bind to multiple different candidate proteins suspected of being present in a given sample, and (2) subjecting the protein sample to a set of promiscuous affinity agents that, taken as a whole, are expected to bind each candidate protein in a different combination, such that each candidate protein is expected to be encoded by a unique profile of binding and non-binding events. Promiscuity of an affinity agent is a characteristic that can be understood relative to a given population of proteins. Promiscuity can arise due to the affinity agent recognizing an epitope that is known to be present in a plurality of different candidate proteins suspected of being present in the given population of unknown proteins. For example, epitopes having relatively short amino acid lengths such as dimers, trimers, or tetramers can be expected to occur in a substantial number of different proteins in the human proteome. Alternatively or additionally, a promiscuous affinity agent can recognize different epitopes (e.g., epitopes differing from each other with regard to amino acid composition or sequence), the different epitopes being present in a plurality of different candidate proteins. For example, a promiscuous affinity agent that is designed or selected for its affinity toward a first trimer epitope may bind to a second epitope that has a different sequence of amino acids when compared to the first epitope.

Although performing a single binding reaction between a promiscuous affinity agent and a complex protein sample may yield ambiguous results regarding the identity of the different proteins to which it binds, the ambiguity can be resolved when the results are combined with other identifying information about those proteins. The identifying information can include characteristics of the protein such as length (i.e. number of amino acids), hydrophobicity, molecular weight, charge to mass ratio, isoelectric point, chromatographic fractionation behavior, enzymatic activity, presence or absence of post translational modifications or the like. The identifying information can include results of binding with other promiscuous affinity agents. For example, a plurality of different promiscuous affinity agents can be contacted with a complex population of proteins, wherein the plurality is configured to produce a different binding profile for each candidate protein suspected of being present in the population. In this example, each of the affinity agents can be distinguishable from the other affinity agents, for example, due to unique labeling (e.g., different affinity agents having different luminophore labels), unique spatial location (e.g., different affinity agents being located at different addresses in an array), and/or unique time of use (e.g., different affinity agents being delivered in series to a population of proteins). Accordingly, the plurality of promiscuous affinity agents produces a binding profile for each individual protein that can be decoded to identify a unique combination of epitopes present in the individual protein, and this can in turn be used to identify the individual protein as a particular candidate protein having the same or similar unique combination of epitopes. The binding profile can include observed binding events as well as observed non-binding events and this information can be evaluated in view of the expectation that particular candidate proteins produce a similar binding profile, for example, based on presence and absence of particular epitopes in the candidate proteins.

In some configurations, distinct and reproducible binding profiles may be observed for one or more unknown proteins in a sample. However, in many cases one or more binding events produces inconclusive or even aberrant results and this, in turn, can yield ambiguous binding profiles. For example, observation of binding outcome for a single-molecule binding event can be particularly prone to ambiguities due to stochasticity in the behavior of single molecules when observed using certain detection hardware. The present disclosure provides methods that provide accurate protein identification despite ambiguities and imperfections that can arise in many contexts. In some configurations, methods for identifying, quantitating, or otherwise characterizing one or more proteins in a sample utilize a binding model that evaluates the likelihood or probability that one or more candidate proteins that are suspected of being present in the sample will have produced an empirically observed binding profile. The binding model can include information regarding expected binding outcomes (e.g., binding or non-binding) for binding of one or more affinity reagent with one or more candidate proteins. The information can include an a priori characteristic of a candidate protein, such as presence or absence of a particular epitope in the candidate protein or length of the candidate protein. Alternatively or additionally, the information can include empirically determined characteristics such as propensity or likelihood that the candidate protein will bind to a particular affinity reagent. Accordingly, a binding model can include information regarding the propensity or likelihood of a given candidate protein generating a false positive or false negative binding result in the presence of a particular affinity reagent, and such information can optionally be included for a plurality of affinity reagents.

Methods set forth herein can be used to evaluate the degree of compatibility of one or more empirical binding profiles with results computed for various candidate proteins using a binding model. For example, to identify an unknown protein in a sample of many proteins, an empirical binding profile for the protein can be compared to results computed by the binding model for many or all candidate proteins suspected of being in the sample. In some configurations of the methods set forth herein, identity for the unknown protein is determined based on a likelihood of the unknown protein being a particular candidate protein given the empirical binding pattern or based on the probability of a particular candidate protein generating the empirical binding pattern. Optionally a score can be determined from the measurements that are acquired for the unknown protein with respect to many or all candidate proteins suspected of being in the sample. A digital or binary score that indicates one of two discrete states can be determined. In particular configurations, the score can be non-digital or non-binary. For example, the score can be a value selected from a continuum of values such that an identity is made based on the score being above or below a threshold value. Moreover, a score can be a single value or a collection of values. Particularly useful methods for identifying proteins using promiscuous reagents, serial binding measurements and/or decoding with binding models are set forth, for example, in U.S. Pat. No. 10,473,654 US Pat. App. Pub. No. 2020/0318101 A1 or Egertson et al., BioRxiv (2021), DOI: 10.1101/2021.10.11.463967, each of which is incorporated herein by reference.

The present disclosure provides compositions, apparatus, and methods for detecting one or more proteins. A protein can be detected using one or more affinity agents having binding affinity for the protein. The affinity agent and the protein can bind each other to form a complex and, during or after formation, the complex can be detected. The complex can be detected directly, for example, due to a label that is present on the affinity agent or protein. In some configurations, the complex need not be directly detected, for example, in formats where the complex is formed and then the affinity agent, protein, or a label component that was present in the complex is detected.

Many protein detection methods, such as enzyme linked immunosorbent assay (ELISA), achieve high-confidence characterization of one or more protein in a sample by exploiting high specificity binding of antibodies, aptamers or other binding agents to the protein(s) and detecting the binding event while ignoring all other proteins in the sample. ELISA is generally carried out at low plex scale (e.g., from one to a hundred different proteins detected in parallel or in succession) but can be used at higher plexity. ELISA methods can be carried out by detecting immobilized binding agents and/or proteins in multiwell plates, on arrays, or on particles in microfluidic devices. Exemplary plate-based methods include, for example, the MULTI-ARRAY technology commercialized by MesoScale Diagnostics (Rockville, Maryland) or Simple Plex technology commercialized by Protein Simple (San Jose, CA). Exemplary, array-based methods include, but are not limited to those utilizing Simoa® Planar Array Technology or Simoa® Bead Technology, commercialized by Quanterix (Billerica, MA). Further exemplary array-based methods are set forth in U.S. Pat. Nos. 9,678,068; 9,395,359; 8,415,171; 8,236,574; or 8,222,047, each of which is incorporated herein by reference. Exemplary microfluidic detection methods include those commercialized by Luminex (Austin, Texas) under the trade name xMAP® technology or used on platforms identified as MAGPIX®, LUMINEX® 100/200 or FEXMAP 3D®.

Other detection methods that can also be used, for example at low plex scale, include procedures that employ SOMAmer reagents and SOMAscan assays commercialized by Soma Logic (Boulder, CO). In one configuration, a sample is contacted with aptamers that are capable of binding proteins with specificity for the amino acid sequence of the proteins. The resulting aptamer-protein complexes can be separated from other sample components, for example, by attaching the complexes to beads (or other solid support) that are removed from other sample components. The aptamers can then be isolated and, because the aptamers are nucleic acids, the aptamers can be detected using any of a variety of methods known in the art for detecting nucleic acids, including for example, hybridization to nucleic acid arrays, PCR-based detection, or nucleic acid sequencing. Exemplary methods and compositions are set forth in U.S. Pat. Nos. 7,855,054; 7,964,356; 8,404,830; 8,945,830; 8,975,026; 8,975,388; 9,163,056; 9,938,314; 9,404,919; 9,926,566; 10,221,421; 10,239,908; 10,316,321 10,221,207 or 10,392,621, each of which is incorporated herein by reference.

In some detection assays, a protein can be cyclically modified and the modified products from individual cycles can be detected. In some configurations, a protein can be sequenced by a sequential process in which each cycle includes steps of detecting the protein and removing one or more terminal amino acids from the protein. Optionally, one or more of the steps can include adding a label to the protein, for example, at the amino terminal amino acid or at the carboxy terminal amino acid. In particular configurations, a method of detecting a protein can include steps of (i) exposing a terminal amino acid on the protein; (ii) detecting a change in signal from the protein; and (iii) identifying the type of amino acid that was removed based on the change detected in step (ii). The terminal amino acid can be exposed, for example, by removal of one or more amino acids from the amino terminus or carboxyl terminus of the protein. Steps (i) through (iii) can be repeated to produce a series of signal changes that is indicative of the sequence for the protein.

In a first configuration of a cyclical protein detection method, one or more types of amino acids in the protein can be attached to a label that uniquely identifies the type of amino acid. In this configuration, the change in signal that identifies the amino acid can be loss of signal from the respective label. For example, lysines can be attached to a distinguishable label such that loss of the label indicates removal of a lysine. Alternatively or additionally, other amino acid types can be attached to other labels that are mutually distinguishable from lysine and from each other. For example, lysines can be attached to a first label and cysteines can be attached to a second label, the first and second labels being distinguishable from each other. Exemplary compositions and techniques that can be used to remove amino acids from a protein and detect signal changes are those set forth in Swaminathan et al., Nature Biotech. 36:1076-1082 (2018); or U.S. Pat. Nos. 9,625,469 or 10,545,153, each of which is incorporated herein by reference. Methods and apparatus under development by Erisyon, Inc. (Austin, TX) may also be useful for detecting proteins.

In a second configuration of a cyclical protein detection method, a terminal amino acid of a protein can be recognized by an affinity agent that is specific for the terminal amino acid or specific for a label moiety that is present on the terminal amino acid. The affinity agent can be detected on the array, for example, due to a label on the affinity agent. Optionally, the label is a nucleic acid barcode sequence that is added to a primer nucleic acid upon formation of a complex. For example, a barcode can be added to the primer via ligation of an oligonucleotide having the barcode sequence or polymerase extension directed by a template that encodes the barcode sequence. The formation of the complex and identity of the terminal amino acid can be determined by decoding the barcode sequence. Multiple cycles can produce a series of barcodes that can be detected, for example, using a nucleic acid sequencing technique. Exemplary affinity agents and detection methods are set forth in US Pat. App. Pub. No. 2019/0145982 A1; 2020/0348308 A1; or 2020/0348307 A1, each of which is incorporated herein by reference. Methods and apparatus under development by Encodia, Inc. (San Diego, CA) may also be useful for detecting proteins.

Cyclical removal of terminal amino acids from a protein can be carried out using an Edman-type sequencing reaction in which a phenyl isothiocyanate reacts with a N-terminal amino group under mildly alkaline conditions (e.g., about pH 8) to form a cyclical phenylthiocarbamoyl Edman complex derivative. The phenyl isothiocyanate may be substituted or unsubstituted with one or more functional groups, linker groups, or linker groups containing functional groups. An Edman-type sequencing reaction can include variations to reagents and conditions that yield a detectable removal of amino acids from a protein terminus, thereby facilitating determination of the amino acid sequence for a protein or portion thereof. For example, the phenyl group can be replaced with at least one aromatic, heteroaromatic or aliphatic group which may participate in an Edman-type sequencing reaction, non-limiting examples including: pyridine, pyrimidine, pyrazine, pyridazoline, fused aromatic groups such as naphthalene and quinoline), methyl or other alkyl groups or alkyl group derivatives (e.g., alkenyl, alkynyl, cyclo-alkyl). Under certain conditions, for example, acidic conditions of about pH 2, derivatized terminal amino acids may be cleaved, for example, as a thiazolinone derivative. The thiazolinone amino acid derivative under acidic conditions may form a more stable phenylthiohydantoin (PTH) or similar amino acid derivative which can be detected. This procedure can be repeated iteratively for residual protein to identify the subsequent N-terminal amino acid. Many variations of Edman-type degradation have been described and may be used including, for example, a one-step removal of an N-terminal amino acid using alkaline conditions (Chang, J. Y., FEBS LETTS., 1978, 91 (1), 63-68). In some cases, Edman-type reactions may be thwarted by N-terminal modifications which may be selectively removed, for example, N-terminal acetylation or formylation (e.g., see Gheorghe M. T., Bergman T. (1995) in Methods in Protein Structure Analysis, Chapter 8: Deacetylation and internal cleavage of Proteins for N-terminal Sequence Analysis. Springer, Boston, MA. https://doi.org/10.1007/978-1-4899-1031-8_8).

Non-limiting examples of functional groups for substituted phenyl isothiocyanate may include ligands (e.g., biotin and biotin analogs) for known receptors, labels such as luminophores, or reactive groups such as click functionalities (e.g., compositions having an azide or acetylene moiety). The functional group may be a DNA, RNA, peptide or small molecule barcode or other tag which may be further processed and/or detected.

The removal of an amino terminal amino acid using Edman-type processes can utilize at least two main steps, the first step includes reacting an isothiocyanate or equivalent with protein N-terminal residues to form a relatively stable Edman complex, for example, a phenylthiocarbamoyl complex. The second step can include removing the derivatized N-terminal amino acid, for example, via heating. The protein, now having been shortened by one amino acid, may be detected, for example, by contacting the protein with a labeled affinity agent that is complementary to the amino terminus and examining the protein for binding to the agent, or by detecting loss of a label that was attached to the removed amino acid.

Edman-type processes can be carried out in a multiplex format to detect, characterize, or identify a plurality of proteins. A method of detecting a protein can include steps of (i) exposing a terminal amino acid on a protein at an address of an array; (ii) binding an affinity agent to the terminal amino acid, where the affinity agent includes a nucleic acid tag, and where a primer nucleic acid is present at the address; (iii) extending the primer nucleic acid, thereby producing an extended primer having a copy of the tag; and (iv) detecting the tag of the extended primer. The terminal amino acid can be exposed, for example, by removal of one or more amino acids from the amino terminus or carboxyl terminus of the protein. Steps (i) through (iv) can be repeated to produce a series of tags that is indicative of the sequence for the protein. The method can be applied to a plurality of proteins on the array and in parallel. Whatever the plexity, the extending of the primer can be carried out, for example, by polymerase-based extension of the primer, using the nucleic acid tag as a template. Alternatively, the extending of the primer can be carried out, for example, by ligase- or chemical-based ligation of the primer to a nucleic acid that is hybridized to the nucleic acid tag. The nucleic acid tag can be detected via hybridization to nucleic acid probes (e.g., in an array), amplification-based detections (e.g., PCR-based detection, or rolling circle amplification-based detection) or nuclei acid sequencing (e.g., cyclical reversible terminator methods, nanopore methods, or single molecule, real time detection methods). Exemplary methods that can be used for detecting proteins using nucleic acid tags are set forth in US Pat. App. Pub. No. 2019/0145982 A1; 2020/0348308 A1; or 2020/0348307 A1, each of which is incorporated herein by reference.

A protein can optionally be detected based on its enzymatic or biological activity. For example, a protein can be contacted with a reactant that is converted to a detectable product by an enzymatic activity of the protein. In other assay formats, a first protein having a known enzymatic function can be contacted with a second protein to determine if the second protein changes the enzymatic function of the first protein. As such, the first protein serves as a reporter system for detection of the second protein. Exemplary changes that can be observed include, but are not limited to, activation of the enzymatic function, inhibition of the enzymatic function, attenuation of the enzymatic function, degradation of the first protein or competition for a reactant or cofactor used by the first protein. Proteins can also be detected based on their binding interactions with other molecules such as proteins, nucleic acids, nucleotides, metabolites, hormones, vitamins, small molecules that participate in biological signal transduction pathways, biological receptors or the like. For example, a protein that participates in a signal transduction pathway can be identified as a particular candidate protein by detecting binding to a second protein that is known to be a binding partner for the candidate protein in the pathway.

The presence or absence of post-translational modifications (PTM) can be detected using a composition, apparatus or method set forth herein. A PTM can be detected using an affinity agent that recognizes the PTM or based on a chemical property of the PTM. Exemplary PTMs that can be detected, identified or characterized include, but are not limited to, myristoylation, palmitoylation, isoprenylation, prenylation, farnesylation, geranylgeranylation, lipoylation, flavin moiety attachment, Heme C attachment, phosphopantetheinylation, retinylidene Schiff base formation, diphthamide formation, ethanolamine phosphoglycerol attachment, hypusine, beta-Lysine addition, acylation, acetylation, deacetylation, formylation, alkylation, methylation, C-terminal amidation, arginylation, polyglutamylation, polyglyclyation, butyrylation, gamma-carboxylation, glycosylation, glycation, polysialylation, malonylation, hydroxylation, iodination, nucleotide addition, phosphonate ester formation, phosphoramidate formation, phosphorylation, adenylylation, uridylylation, propionylation, pyroglutamate formation, S-glutathionylation, S-nitrosylation, S-sulfenylation, S-sulfinylation, S-sulfonylation, succinylation, sulfation, glycation, carbamylation, carbonylation, isopeptide bond formation, biotinylation, carbamylation, oxidation, reduction, pegylation, ISGylation, SUMOylation, ubiquitination, neddylation, pupylation, citrullination, deamidation, eliminylation, disulfide bridge formation, proteolytic cleavage, isoaspartate formation, racemization, and protein splicing.

PTMs may occur at particular amino acid residues of a protein. For example, the phosphate moiety of a particular proteoform can be present on a serine, threonine, tyrosine, histidine, cysteine, lysine, aspartate, or glutamate residue of the protein. In other examples, an acetyl moiety can be present on the N-terminus or on a lysine; a serine or threonine residue can have an O-linked glycosyl moiety; an asparagine residue can have an N-linked glycosyl moiety; a proline, lysine, asparagine, aspartate or histidine amino acid can be hydroxylated; an arginine or lysine residue can be methylated; or the N-terminal methionine or at a lysine amino acid can be ubiquitinated.

In some configurations of the apparatus and methods set forth herein, one or more proteins can be detected on a solid support. For example, protein(s) can be attached to a support, the support can be contacted with detection agents (e.g., affinity agents) in solution, the agents can interact with the protein(s), thereby producing a detectable signal, and then the signal can be detected to determine the presence of the protein(s). In multiplexed versions of this approach, different proteins can be attached to different addresses in an array, and the probing and detection steps can occur in parallel. In another example, affinity agents can be attached to a solid support, the support can be contacted with proteins in solution, the proteins can interact with the affinity agents, thereby producing a detectable signal, and then the signal can be detected to determine presence, quantity, or characteristics of the proteins. This approach can also be multiplexed by attaching different affinity agents to different addresses of an array.

Proteins, affinity agents or other objects of interest can be attached to a solid support via covalent or non-covalent bonds. For example, a linker can be used to covalently attach a protein or other object of interest to an array. A particularly useful linker is a structured nucleic acid particle such as a nucleic acid nanoball (e.g., a concatemeric amplicon produced by rolling circle replication of a circular nucleic acid template) or a nucleic acid origami. For example, a plurality of proteins can be conjugated to a plurality of structured nucleic acid particles, such that each protein-conjugated particle forms an address in the array. Exemplary linkers for attaching proteins, or other objects of interest, to an array or other solid support are set forth in US Pat. App. Pub. No. 2021/0101930 A1, which is incorporated herein by reference.

A protein can be detected based on proximity of two or more affinity agents. For example, the two affinity agents can include two components each: a receptor component and a nucleic acid component. When the affinity agents bind in proximity to each other, for example, due to ligands for the respective receptors being on a single protein, or due to the ligands being present on two proteins that associate with each other, the nucleic acids can interact to cause a modification that is indicative of the two ligands being in proximity. Optionally, the modification can be polymerase catalyzed extension of one of the nucleic acids using the other nucleic acid as a template. As another option, one of the nucleic acids can form a template that acts as splint to position other nucleic acids for ligation to an oligonucleotide. Exemplary methods are commercialized by Olink Proteomics AB (Uppsala Sweden) or set forth in U.S. Pat. Nos. 7,306,904; 7,351,528; 8,013,134; 8,268,554 or 9,777,315, each of which is incorporated herein by reference.

A method or apparatus of the present disclosure can optionally be configured for optical detection (e.g., luminescence detection). Analytes or other entities can be detected, and optionally distinguished from each other, based on measurable characteristics such as the wavelength of radiation that excites a luminophore, the wavelength of radiation emitted by a luminophore, the intensity of radiation emitted by a luminophore (e.g., at particular detection wavelength(s)), luminescence lifetime (e.g., the time that a luminophore remains in an excited state) or luminescence polarity. Other optical characteristics that can be detected, and optionally used to distinguish analytes, include, for example, absorbance of radiation, resonance Raman, radiation scattering, or the like. A luminophore can be an intrinsic moiety of a protein or other analyte to be detected, or the luminophore can be an exogenous moiety that has been synthetically added to a protein or other analyte.

A method or apparatus of the present disclosure can use a light sensing device that is appropriate for detecting a characteristic set forth herein or known in the art. Particularly useful components of a light sensing device can include, but are not limited to, optical sub-systems or components used in nucleic acid sequencing systems. Examples of useful sub systems and components thereof are set forth in US Pat. App. Pub. No. 2010/0111768 A1 or U.S. Pat. Nos. 7,329,860; 8,951,781 or 9,193,996, each of which is incorporated herein by reference. Other useful light sensing devices and components thereof are described in U.S. Pat. Nos. 5,888,737; 6,175,002; 5,695,934; 6,140,489; or 5,863,722; or US Pat. Pub. Nos. 2007/007991 A1, 2009/0247414 A1, or 2010/0111768; or WO2007/123744, each of which is incorporated herein by reference. Light sensing devices and components that can be used to detect luminophores based on luminescence lifetime are described, for example, in U.S. Pat. Nos. 9,678,012; 9,921,157; 10,605,730; 10,712,274; 10,775,305; or 10,895,534, each of which is incorporated herein by reference.

Luminescence lifetime can be detected using an integrated circuit having a photodetection region configured to receive incident photons and produce a plurality of charge carriers in response to the incident photons. The integrated circuit can include at least one charge carrier storage region and a charge carrier segregation structure configured to selectively direct charge carriers of the plurality of charge carriers directly into the charge carrier storage region based upon times at which the charge carriers are produced. See, for example, U.S. Pat. Nos. 9,606,058, 10,775,305, and 10,845,308, each of which is incorporated herein by reference. Optical sources that produce short optical pulses can be used for luminescence lifetime measurements. For example, a light source, such as a semiconductor laser or LED, can be driven with a bipolar waveform to generate optical pulses with FWHM durations as short as approximately 85 ps having suppressed tail emission. See, for example, in U.S. Pat. No. 10,605,730, which is incorporated herein by reference.

For configurations that use optical detection (e.g., luminescent detection), one or more analytes (e.g., proteins) may be immobilized on a surface, and this surface may be scanned with a microscope to detect any signal from the immobilized analytes. The microscope itself may include a digital camera or other luminescence detector configured to record, store, and analyze the data collected during the scan. A luminescence detector of the present disclosure can be configured for epiluminescent detection, total internal reflection (TIR) detection, waveguide assisted excitation, or the like.

A light sensing device may be based upon any suitable technology, and may be, for example, a charged coupled device (CCD) sensor that generates pixilated image data based upon photons impacting locations in the device. It will be understood that any of a variety of other light sensing devices may also be used including, but not limited to, a detector array configured for time delay integration (TDI) operation, a complementary metal oxide semiconductor (CMOS) detector, an avalanche photodiode (APD) detector, a Geiger-mode photon counter, a photomultiplier tube (PMT), charge injection device (CID) sensors, JOT image sensor (Quanta), or any other suitable detector. Light sensing devices can optionally be coupled with one or more excitation sources, for example, lasers, light emitting diodes (LEDs), arc lamps or other energy sources known in the art.

An optical detection system can be configured for single molecule detection. For example, waveguides or optical confinements can be used to deliver excitation radiation to locations of a solid support where analytes are located. Zero-mode waveguides can be particularly useful, examples of which are set forth in U.S. Pat. Nos. 7,181,122, 7,302,146, or 7,313,308, each of which is incorporated herein by reference. Analytes can be confined to surface features, for example, to facilitate single molecule resolution. For example, analytes can be distributed into wells having nanometer dimensions such as those set forth in U.S. Pat. Nos. 7,122,482 or 8,765,359, or US Pat. App. Pub. No 2013/0116153 A1, each of which is incorporated herein by reference. The wells can be configured for selective excitation, for example, as set forth in U.S. Pat. No. 8,798,414 or 9,347,829, each of which is incorporated herein by reference. Analytes can be distributed to nanometer-scale posts, such as high aspect ratio posts which can optionally be dielectric pillars that extend through a metallic layer to improve detection of an analyte attached to the pillar. See, for example, U.S. Pat. Nos. 8,148,264, 9,410,887 or 9,987,609, each of which is incorporated herein by reference. Further examples of nanostructures that can be used to detect analytes are those that change state in response to the concentration of analytes such that the analytes can be quantitated as set forth in WO 2020/176793 A1, which is incorporated herein by reference.

An apparatus or method set forth herein need not be configured for optical detection. For example, an electronic detector can be used for detection of protons or charged labels (see, for example, US Pat. App. Pub. Nos. 2009/0026082 A1; 2009/0127589 A1; 2010/0137143 A1; or 2010/0282617 A1, each of which is incorporated herein by reference in its entirety). A field effect transistor (FET) can be used to detect analytes or other entities, for example, based on proximity of a field disrupting moiety to the FET. The field disrupting moiety can be due to an extrinsic label attached to an analyte or affinity agent, or the moiety can be intrinsic to the analyte or affinity agent being used. Surface plasmon resonance can be used to detect binding of analytes or affinity agents at or near a surface. Exemplary sensors and methods for attaching molecules to sensors are set forth in US Pat. App. Pub. Nos. 2017/0240962 A1; 2018/0051316 A1; 2018/0112265 A1; 2018/0155773 A1 or 2018/0305727 A1; or U.S. Pat. Nos. 9,164,053; 9,829,456; 10,036,064, each of which is incorporated herein by reference.

In some configurations of the compositions, apparatus and methods set forth herein, one or more proteins can be present on a solid support, where the proteins can optionally be detected. For example, a protein can be attached to a solid support, the solid support can be contacted with a detection agent (e.g., affinity agent) in solution, the affinity agent can interact with the protein, thereby producing a detectable signal, and then the signal can be detected to determine the presence, absence, quantity, a characteristic, or identity of the protein. In multiplexed versions of this approach, different proteins can be attached to different addresses in an array, and the detection steps can occur in parallel, such that proteins at each address are detected, quantified, characterized, or identified. In another example, detection agents can be attached to a solid support, the support can be contacted with proteins in solution, the proteins can interact with the detection agents, thereby producing a detectable signal, and then the signal can be detected to determine the presence of the proteins. This approach can also be multiplexed by attaching different probes to different addresses of an array.

In multiplexed configurations, different proteins can be attached to different unique identifiers (e.g., addresses in an array), and the proteins can be manipulated and detected in parallel. For example, a fluid containing one or more different affinity agents can be delivered to an array such that the proteins of the array are in simultaneous contact with the affinity agent(s). Moreover, a plurality of addresses can be observed in parallel allowing for rapid detection of binding events. A plurality of different proteins can have a complexity of at least 5, 10, 100, 1×10³, 1×10⁴, 1×10⁵ or more different native-length protein primary sequences. Alternatively or additionally, a proteome, proteome subfraction or other protein sample that is analyzed in a method set forth herein can have a complexity that is at most 1×10⁵, 1×10⁴, 1×10³, 100, 10, 5 or fewer different native-length protein primary sequences. The total number of proteins of a sample that is detected, characterized, or identified can differ from the number of different primary sequences in the sample, for example, due to the presence of multiple copies of at least some protein species. Moreover, the total number of proteins of a sample that is detected, characterized, or identified can differ from the number of candidate proteins suspected of being in the sample, for example, due to the presence of multiple copies of at least some protein species, absence of some proteins in a source for the sample, or loss of some proteins prior to analysis.

A protein can be attached to a unique identifier using any of a variety of means. The attachment can be covalent or non-covalent. Exemplary covalent attachments include chemical linkers such as those achieved using click chemistry or other linkages known in the art or described in U.S. patent application Ser. No. 17/062,405, which is incorporated herein by reference. Non-covalent attachment can be mediated by receptor-ligand interactions (e.g., (strept) avidin-biotin, antibody-antigen, or complementary nucleic acid strands), for example, wherein the receptor is attached to the unique identifier and the ligand is attached to the protein or vice versa. In particular configurations, a protein is attached to a solid support (e.g., an address in an array) via a structured nucleic acid particle (SNAP). A protein can be attached to a SNAP and the SNAP can interact with a solid support, for example, by non-covalent interactions of the DNA with the support and/or via covalent linkage of the SNAP to the support. Nucleic acid origami or nucleic acid nanoballs are particularly useful. The use of SNAPs and other moieties to attach proteins to unique identifiers such as tags or addresses in an array are set forth in U.S. patents application Ser. Nos. 17/062,405 and 63/159,500, each of which is incorporated herein by reference.

The methods, compositions and apparatus of the present disclosure are particularly well suited for use with proteins. Although proteins are exemplified throughout the present disclosure, it will be understood that other analytes can be similarly used. Exemplary analytes include, but are not limited to, biomolecules, polysaccharides, nucleic acids, lipids, metabolites, hormones, vitamins, enzyme cofactors, therapeutic agents, candidate therapeutic agents or combinations thereof. An analyte can be a non-biological atom or molecule, such as a synthetic polymer, metal, metal oxide, ceramic, semiconductor, mineral, or a combination thereof.

One or more proteins that are used in a method, composition, or apparatus herein, can be derived from a natural or synthetic source. Exemplary sources include, but are not limited to biological tissues, fluids, cells, or subcellular compartments (e.g., organelles). For example, a sample can be derived from a tissue biopsy, biological fluid (e.g., blood, sweat, tears, plasma, extracellular fluid, urine, mucus, saliva, semen, vaginal fluid, synovial fluid, lymph, cerebrospinal fluid, peritoneal fluid, pleural fluid, amniotic fluid, intracellular fluid, extracellular fluid, etc.), fecal sample, hair sample, cultured cell, culture media, fixed tissue sample (e.g., fresh frozen or formalin-fixed paraffin-embedded) or product of a protein synthesis reaction. A protein source may include any sample where a protein is a native or expected constituent. For example, a primary source for a cancer biomarker protein may be a tumor biopsy sample or bodily fluid. Other sources include environmental samples or forensic samples.

Exemplary organisms from which proteins or other analytes can be derived include, for example, a mammal such as a rodent, mouse, rat, rabbit, guinea pig, ungulate, horse, sheep, pig, goat, cow, cat, dog, primate, non-human primate or human; a plant such as Arabidopsis thaliana, tobacco, corn, sorghum, oat, wheat, rice, canola, or soybean; an algae such as Chlamydomonas reinhardtii; a nematode such as Caenorhabditis elegans; an insect such as Drosophila melanogaster, mosquito, fruit fly, honey bee or spider; a fish such as zebrafish; a reptile; an amphibian such as a frog or Xenopus laevis; a Dictyostelium discoideum; a fungi such as Pneumocystis carinii, Takifugu rubripes, yeast, Saccharomyces cerevisiae or Schizosaccharomyces pombe; or a Plasmodium falciparum. Proteins can also be derived from a prokaryote such as a bacterium, Escherichia coli, staphylococci, or Mycoplasma pneumoniae; an archae; a virus such as Hepatitis C virus, influenza virus, coronavirus, or human immunodeficiency virus; or a viroid. Proteins can be derived from a homogeneous culture or population of the above organisms or alternatively from a collection of several different organisms, for example, in a community or ecosystem.

In some cases, a protein or other biomolecule can be derived from an organism that is collected from a host organism. For example, a protein may be derived from a parasitic, pathogenic, symbiotic, or latent organism collected from a host organism. A protein can be derived from an organism, tissue, cell, or biological fluid that is known or suspected of being linked with a disease state or disorder (e.g., cancer). Alternatively, a protein can be derived from an organism, tissue, cell, or biological fluid that is known or suspected of not being linked to a particular disease state or disorder. For example, the proteins isolated from such a source can be used as a control for comparison to results acquired from a source that is known or suspected of being linked to the particular disease state or disorder. A sample may include a microbiome or substantial portion of a microbiome. In some cases, one or more proteins used in a method, composition or apparatus set forth herein may be obtained from a single source and no more than the single source. The single source can be, for example, a single organism (e.g., an individual human), single tissue, single cell, single organelle (e.g., endoplasmic reticulum, Golgi apparatus or nucleus), or single protein-containing particle (e.g., a viral particle or vesicle).

A method, composition or apparatus of the present disclosure can use or include a plurality of proteins having any of a variety of compositions such as a plurality of proteins composed of a proteome or fraction thereof. For example, a plurality of proteins can include solution-phase proteins, such as proteins in a biological sample or fraction thereof, or a plurality of proteins can include proteins that are immobilized, such as proteins attached to a particle or solid support. By way of further example, a plurality of proteins can include proteins that are detected, analyzed, or identified in connection with a method, composition, or apparatus of the present disclosure. The content of a plurality of proteins can be understood according to any of a variety of characteristics such as those set forth below or elsewhere herein.

A plurality of proteins can be characterized in terms of total protein mass. The total mass of protein in a liter of plasma has been estimated to be 70 g and the total mass of protein in a human cell has been estimated to be between 100 pg and 500 pg depending upon cells type. See Wisniewski et al., Molecular & Cellular Proteomics 13:10.1074/mcp.M113.037309, 3497-3506 (2014), which is incorporated herein by reference. A plurality of proteins used or included in a method, composition or apparatus set forth herein can include at least 1 pg, 10 pg, 100 pg, 1 ng, 10 ng, 100 ng, 1 μg, 10 μg, 100 μg, 1 mg, 10 mg, 100 mg or more protein by mass. Alternatively or additionally, a plurality of proteins may contain at most 100 mg, 10 mg, 1 mg, 100 μg, 10 μg, 1 μg, 100 ng, 10 ng, 1 ng, 100 pg, 10 pg, 1 pg or less protein by mass.

A plurality of proteins can be characterized in terms of percent mass relative to a given source such as a biological source (e.g., cell, tissue, or biological fluid such as blood). For example, a plurality of proteins may contain at least 60%, 75%, 90%, 95%, 99%, 99.9% or more of the total protein mass present in the source from which the plurality of proteins was derived. Alternatively or additionally, a plurality of proteins may contain at most 99.9%, 99%, 95%, 90%, 75%, 60% or less of the total protein mass present in the source from which the plurality of proteins was derived.

A plurality of proteins can be characterized in terms of total number of protein molecules. The total number of protein molecules in a Saccharomyces cerevisiae cell has been estimated to be about 42 million protein molecules. See Ho et al., Cell Systems (2018), DOI: 10.1016/j.cels.2017.12.004, which is incorporated herein by reference. A plurality of proteins used or included in a method, composition or apparatus set forth herein can include at least 1 protein molecule, 10 protein molecules, 100 protein molecules, 1×10⁴ protein molecules, 1×10⁶ protein molecules, 1×10⁸ protein molecules, 1×10¹⁰ protein molecules, 1 mole (6.02214076×10²³ molecules) of protein, 10 moles of protein molecules, 100 moles of protein molecules or more. Alternatively or additionally, a plurality of proteins may contain at most 100 moles of protein molecules, 10 moles of protein molecules, 1 mole of protein molecules, 1×10¹⁰ protein molecules, 1×10⁸ protein molecules, 1×10⁶ protein molecules, 1×10⁴ protein molecules, 100 protein molecules, 10 protein molecules, 1 protein molecule or less.

A plurality of proteins can be characterized in terms of the variety of full-length primary protein structures in the plurality. For example, the variety of full-length primary protein structures in a plurality of proteins can be equated with the number of different protein-encoding genes in the source for the plurality of proteins. Whether or not the proteins are derived from a known genome or from any genome at all, the variety of full-length primary protein structures can be counted independent of presence or absence of post translational modifications in the proteins. A human proteome is estimated to have about 20,000 different protein-encoding genes such that a plurality of proteins derived from a human can include up to about 20,000 different primary protein structures. See Aebersold et al., Nat. Chem. Biol. 14:206-214 (2018), which is incorporated herein by reference. Other genomes and proteomes in nature are known to be larger or smaller. A plurality of proteins used or included in a method, composition or apparatus set forth herein can have a complexity of at least 2, 5, 10, 100, 1×10³, 1×10⁴, 2×10⁴, 3×10⁴ or more different full-length primary protein structures. Alternatively or additionally, a plurality of proteins can have a complexity that is at most 3×10⁴, 2×10⁴, 1×10⁴, 1×10³, 100, 10, 5, 2 or fewer different full-length primary protein structures.

In relative terms, a plurality of proteins used or included in a method, composition or apparatus set forth herein may contain at least one representative for at least 60%, 75%, 90%, 95%, 99%, 99.9% or more of the proteins encoded by the genome of a source from which the sample was derived. Alternatively or additionally, a plurality of proteins may contain a representative for at most 99.9%, 99%, 95%, 90%, 75%, 60% or less of the proteins encoded by the genome of a source from which the sample was derived.

A plurality of proteins can be characterized in terms of the variety of primary protein structures in the plurality including transcribed splice variants. The human proteome has been estimated to include about 70,000 different primary protein structures when splice variants ae included. See Aebersold et al., Nat. Chem. Biol. 14:206-214 (2018), which is incorporated herein by reference. Moreover, the number of the partial-length primary protein structures can increase due to fragmentation that occurs in a sample. A plurality of proteins used or included in a method, composition or apparatus set forth herein can have a complexity of at least 2, 5, 10, 100, 1×10³, 1×10⁴, 7×10⁴, 1×10⁵, 1×10⁶ or more different primary protein structures. Alternatively or additionally, a plurality of proteins can have a complexity that is at most 1×10⁶, 1×10⁵, 7×10⁴, 1×10⁴, 1×10³, 100, 10, 5, 2 or fewer different primary protein structures.

A plurality of proteins can be characterized in terms of the variety of protein structures in the plurality including different primary structures and different proteoforms among the primary structures. Different molecular forms of proteins expressed from a given gene are considered to be different proteoforms. Proteoforms can differ, for example, due to differences in primary structure (e.g., shorter or longer amino acid sequences), different arrangement of domains (e.g., transcriptional splice variants), or different post translational modifications (e.g., presence or absence of phosphoryl, glycosyl, acetyl, or ubiquitin moieties). The human proteome is estimated to include hundreds of thousands of proteins when counting the different primary structures and proteoforms. See Aebersold et al., Nat. Chem. Biol. 14:206-214 (2018), which is incorporated herein by reference. A plurality of proteins used or included in a method, composition or apparatus set forth herein can have a complexity of at least 2, 5, 10, 100, 1×10³, 1×10⁴, 1×10⁵, 1×10⁶, 5×10⁶, 1×10⁷ or more different protein structures. Alternatively or additionally, a plurality of proteins can have a complexity that is at most 1×10⁷, 5×10⁶, 1×10⁶, 1×10⁵, 1×10⁴, 1×10³, 100, 10, 5, 2 or fewer different protein structures.

A plurality of proteins can be characterized in terms of the dynamic range for the different protein structures in the sample. The dynamic range can be a measure of the range of abundance for all different protein structures in a plurality of proteins, the range of abundance for all different primary protein structures in a plurality of proteins, the range of abundance for all different full-length primary protein structures in a plurality of proteins, the range of abundance for all different full-length gene products in a plurality of proteins, the range of abundance for all different proteoforms expressed from a given gene, or the range of abundance for any other set of different proteins set forth herein. The dynamic range for all proteins in human plasma is estimated to span more than 10 orders of magnitude from albumin, the most abundant protein, to the rarest proteins that have been measured clinically. See Anderson and Anderson Mol Cell Proteomics 1:845-67 (2002), which is incorporated herein by reference. The dynamic range for plurality of proteins set forth herein can be a factor of at least 10, 100, 1×10³, 1×10⁴, 1×10⁶, 1×10⁸, 1×10¹⁰, or more. Alternatively or additionally, the dynamic range for plurality of proteins set forth herein can be a factor of at most 1×10¹⁰, 1×10⁸, 1×10⁶, 1×10⁴, 1×10³, 100, 10 or less.

A method set forth herein can be carried out in a fluid phase or on a solid phase. For fluid phase configurations, a fluid containing one or more proteins can be mixed with another fluid containing one or more affinity agents. For solid phase configurations one or more proteins or affinity agents can be attached to a solid support. One or more components that will participate in a binding event can be contained in a fluid and the fluid can be delivered to a solid support, the solid support being attached to one or more other component that will participate in the binding event.

A method of the present disclosure can be carried out at single analyte resolution. Alternatively to single-analyte resolution, a method set forth herein can be carried out at ensemble-resolution or bulk-resolution. Bulk-resolution configurations acquire a composite signal from a plurality of different analytes or affinity agents in a vessel or on a surface. For example, a composite signal can be acquired from a population of different protein-affinity agent complexes in a well or cuvette, or on a solid support surface, such that individual complexes are not resolved from each other. Ensemble-resolution configurations acquire a composite signal from a first collection of proteins or affinity agents in a sample, such that the composite signal is distinguishable from signals generated by a second collection of proteins or affinity agents in the sample. For example, the ensembles can be located at different addresses in an array. Accordingly, the composite signal obtained from each address will be an average of signals from the ensemble, yet signals from different addresses can be distinguished from each other.

A composition, apparatus or method set forth herein can be configured to contact one or more proteins (e.g., an array of different proteins) with a plurality of different affinity agents. For example, a plurality of affinity agents (whether configured separately or as a pool) may include at least 2, 5, 10, 25, 50, 100, 250, 500 or more types of affinity agents, each type of affinity agent differing from the other types with respect to the epitope(s) recognized. Alternatively or additionally, a plurality of affinity agents may include at most 500, 250, 100, 50, 25, 10, 5, or 2 types of affinity agents, each type of affinity agent differing from the other types with respect to the epitope(s) recognized. Different types of affinity agents in a pool can be uniquely labeled such that the different types can be distinguished from each other. In some configurations, at least two, and up to all, of the different types of affinity agents in a pool may be indistinguishably labeled with respect to each other. Alternatively or additionally to the use of unique labels, different types of affinity agents can be delivered and detected serially when evaluating one or more proteins (e.g., in an array).

A method of the present disclosure can be performed in a multiplex format. In multiplexed configurations, different proteins can be attached to different unique identifiers (e.g., the proteins can be attached to different addresses in an array). Multiplexed proteins can be manipulated and detected in parallel. For example, a fluid containing one or more different affinity agents can be delivered to a protein array such that the proteins of the array are in simultaneous contact with the affinity agent(s). Moreover, a plurality of addresses can be observed in parallel allowing for rapid detection of binding events. A plurality of different proteins can have a complexity of at least 5, 10, 100, 1×10³, 1×10⁴, 2×10⁴, 3×10⁴ or more different native-length protein primary sequences. Alternatively or additionally, a proteome or proteome subfraction that is analyzed in a method set forth herein can have a complexity that is at most 3×10⁴, 2×10⁴, 1×10⁴, 1×10³, 100, 10, 5 or fewer different native-length protein primary sequences. The plurality of proteins can constitute a proteome or subfraction of a proteome. The total number of proteins that is detected, characterized, or identified can differ from the number of different primary sequences in the sample from which the proteins are derived, for example, due to the presence of multiple copies of at least some protein species. Moreover, the total number of proteins that are detected, characterized, or identified can differ from the number of candidate proteins suspected of being present, for example, due to the presence of multiple copies of at least some protein species, absence of some proteins in a source for the proteins, or loss of some proteins prior to analysis.

A particularly useful multiplex format uses an array of proteins and/or affinity agents. A polypeptide, anchoring group, polypeptide composite or other analyte can be attached to a unique identifier, such as an address in an array, using any of a variety of means. The attachment can be covalent or non-covalent. Exemplary covalent attachments include chemical linkers such as those achieved using click chemistry or other linkages known in the art or described in US Pat. App. Pub. No. 2021/0101930 A1, which is incorporated herein by reference. Non-covalent attachment can be mediated by receptor-ligand interactions (e.g., (strept) avidin-biotin, antibody-antigen, or complementary nucleic acid strands), for example, in which the receptor is attached to the unique identifier and the ligand is attached to the protein or vice versa. In particular configurations, a protein is attached to a solid support (e.g., an address in an array) via a structured nucleic acid particle (SNAP). A protein can be attached to a SNAP and the SNAP can interact with a solid support, for example, by non-covalent interactions of the DNA with the support and/or via covalent linkage of the SNAP to the support. Nucleic acid origami or nucleic acid nanoballs are particularly useful. The use of SNAPs and other moieties to attach proteins to unique identifiers such as tags or addresses in an array are set forth in US Pat. App. Pub. No. 2021/0101930 A1, which is incorporated herein by reference.

A solid support or a surface thereof may be configured to display an analyte or a plurality of analytes. A solid support may contain one or more patterned, formed, or prepared surfaces that contain at least one address for displaying an analyte. In some cases, a solid support may contain one or more patterned, formed, or prepared surfaces that contain a plurality of addresses, with each address configured to display one or more analytes. Accordingly, an array as set forth herein may comprise a plurality of analytes coupled to a solid support or a surface thereof. In some configurations, a solid support or a surface thereof may be patterned or formed to produce an ordered or patterned array of addresses. The deposition of analytes on the ordered or patterned array of addresses may be controlled by interactions between the solid support and the analytes such as, for example, electrostatic interactions, magnetic interactions, hydrophobic interactions, hydrophilic interactions, covalent interactions, or non-covalent interactions. Accordingly, the coupling of an analyte at each address of an array may produce an ordered or patterned array of analytes whose average spacing between analytes is determined based upon the tolerance of the ordering or patterning of the solid support and the size of an analyte-binding region for each address. An ordered or patterned array of analytes may be characterized as having a regular geometry, such as a rectangular, triangular, polygonal, or annular grid. In other configurations, a solid support or a surface thereof may be non-patterned or non-ordered. The deposition of analytes on the non-ordered or non-patterned array of addresses may be controlled by interactions between the solid support and the analytes, or inter-analyte interactions such as, for example, steric repulsion, electrostatic repulsion, electrostatic attraction, magnetic repulsion, magnetic attraction, covalent interactions, or non-covalent interactions.

A solid support or a surface thereof may contain one or more structures or features. A structure or feature may comprise an elevation, profile, shape, geometry, or configuration that deviates from an average elevation, profile, shape, geometry, or configuration of a solid support or surface thereof. A structure or feature may be a raised structure or feature, such as a ridge, post, pillar, or pad, if the structure or feature extends above the average elevation of a surface of a solid support. A structure or feature may be a depressed structure, such as a channel, well, pore, or hole, if the structure or feature extends below the average elevation of a surface of a solid support. A structure or feature may be an intrinsic structure or feature of a substrate (i.e., arising due to the physical or chemical properties of the substrate, or a physical or chemical mechanism of formation), such as surface roughness structures, crystal structures, or porosity. A structure or feature may be formed by a method of processing a solid support. In some configurations, a solid support or a surface may be processed by a lithographic method to form one or more structures or features. A solid support or a surface thereof may be formed by a suitable lithographic method, including, but not limited to photolithography, Dip-Pen nanolithography, nanoimprint lithography, nanosphere lithography, nanoball lithography, nanopillar arrays, nanowire lithography, immersion lithography, neutral particle lithography, plasmonic lithography, scanning probe lithography, thermochemical lithography, thermal scanning probe lithography, local oxidation nanolithography, molecular self-assembly, stencil lithography, laser interference lithography, soft lithography, magnetolithography, stereolithography, deep ultraviolet lithography, x-ray lithography, ion projection lithography, proton-beam lithography, or electron-beam lithography.

A solid support or surface may comprise a plurality of structures or features. A plurality of structures or features may comprise an ordered or patterned array of structures or features. A plurality of structures or features may comprise a non-ordered, non-patterned, or random array of structures or features. A structure or feature may have an average characteristic dimension (e.g., length, width, height, diameter, circumference, etc.) of at least about 1 nanometer (nm), 5 nm, 10 nm, 20 nm, 30 nm, 40 nm, 50 nm, 75 nm, 100 nm, 150 nm, 200 nm, 250 nm, 300 nm, 400 nm, 500 nm, 750 nm, 1000 nm, or more than 1000 nm. Alternatively or additionally, a structure or feature may have an average characteristic dimension of no more than about 1000 nm, 750 nm, 500 nm, 400 nm, 300 nm, 250 nm, 200 nm, 150 nm, 100 nm, 75 nm, 50 nm, 40 nm, 30 nm, 20 nm, 10 nm, 5 nm, 1 nm, or less than 1 nm. An array of structures or features may have an average pitch, in which the pitch is measured as the average separation between respective centerpoints of neighboring structures or features. An array may have an average pitch of at least about 1 nm, 5 nm, 10 nm, 20 nm, 30 nm, 40 nm, 50 nm, 75 nm, 100 nm, 150 nm, 200 nm, 250 nm, 300 nm, 400 nm, 500 nm, 750 nm, 1 micron (μm), 2 μm, 5 μm, 10 μm, 50 μm, 100 μm, or more than 100 μm. Alternatively or additionally, an array may have an average pitch of no more than about 100 μm, 50 μm, 10 μm, 5 μm, 1 μm, 750 nm, 500 nm, 400 nm, 300 nm, 250 nm, 200 nm, 150 nm, 100 nm, 75 nm, 50 nm, 40 nm, 30 nm, 20 nm, 10 nm, 5 nm, 1 nm, or less than 1 nm.

A solid support or a surface thereof may include a base substrate material and, optionally, one or more additional materials that are contacted or adhered with the substrate material. A solid support may comprise one or more additional materials that are deposited, coated, or inlayed onto the substrate material. Additional materials may be added to the substrate material to alter the properties of the substrate material. For example, materials may be added to alter the surface chemistry (e.g., hydrophobicity, hydrophilicity, non-specific binding, electrostatic properties), alter the optical properties (e.g., reflective properties, refractive properties), alter the electrical or magnetic properties (e.g., dielectric materials, conducting materials, electrically-insulating materials), or alter the heat transfer characteristics of the substrate material. Additional materials contacted or adhered with a substrate material may be ordered or patterned onto the substrate material to, for example, locate the additional material at addresses or locate the additional material at interstitial regions between addresses. Exemplary additional materials may include metals (e.g., gold, silver, copper, etc.), metal oxides (e.g., titanium oxide, silicon dioxide, alumina, iron oxides, etc.), metal nitrides (e.g., silicon nitride, aluminum nitride, boron nitride, gallium nitride, etc.), metal carbides (e.g., tungsten carbide, titanium carbide, iron carbide, etc.), metal sulfides (e.g., iron sulfide, silver sulfide, etc.), and organic moieties (e.g., polyethylene glycol (PEG), dextrans, chemically-reactive functional groups, etc.).

A method of the present disclosure can include the step of coupling one or more analytes to a solid support or a surface thereof prior to performing a detection step set forth herein. The coupling of one or more analytes to a solid support surface may include covalent or non-covalent coupling of the one or more analytes to the solid support. Covalent coupling of an analyte to a solid support can include direct covalent coupling of an analyte to a solid support (e.g., formation of coordination bonds) or indirect covalent coupling between a reactive functional group of the analyte and a reactive functional group that is coupled to the solid support (e.g., a CLICK-type reaction). Non-covalent coupling can include the formation of any non-covalent interaction between an analyte and a solid support, including electrostatic or magnetic interactions, or non-covalent bonding interactions (e.g., ionic bonds, van der Waals interactions, hydrogen bonding, etc.). The skilled person will readily recognize that the particular analyte and the choice of solid support can affect the selection of a coupling chemistry for the compositions and methods set forth herein.

Accordingly, a coupling chemistry may be selected based upon the criterium that it provides a sufficiently stable coupling of an analyte to a solid support for a time scale that meets or exceeds the time scale of a method as set forth herein. For example, a polypeptide identification method can require a coupling of the analyte to the solid support for a sufficient amount of time to permit a series of empirical measurements of the analyte to occur. An analyte may be continuously coupled to a solid support for an observable length of time such as, for example, at least about 1 minute, 1 hour (hr), 3 hrs, 6 hrs, 12 hrs, 1 day, 1.5 days, 2 days, 3 days, 1 week (wk), 2 wks, 3 wks, 1 month, or more. The coupling of an analyte to a solid support can occur with a solution-phase chemistry that promotes the deposition of the analyte on the solid support. Coupling of an analyte to a solid support may occur under solution conditions that are optimized for any conceivable solution property, including solution composition, species concentrations, pH, ionic strength, solution temperature, etc. Solution composition can be varied by chemical species, such as buffer type, salts, acids, bases, and surfactants. In some configurations, species such as salts and surfactants may be selected to facilitate the formation of interactions between an analyte and a solid support. Covalent coupling methods for coupling an analyte to a solid support may include species such as catalyst, initiators, and promoters to facilitate particular reactive chemistries.

Coupling of an analyte to a solid support may be facilitated by a mediating group. A mediating group may modify the properties of the analyte to facilitate the coupling. Useful mediating groups have been set forth herein (e.g., structured nucleic acid particles). In some configurations, a mediating group can be coupled to an analyte prior to coupling the analyte to a solid support. Accordingly, the mediating group may be chosen to increase the strength, control, or specificity of the coupling of the analyte to the solid support. In other configurations, a mediating group can be coupled to a solid support prior to coupling an analyte to the solid support. Accordingly, the mediating group may be chosen to provide a more favorable coupling chemistry than can be provided by the solid support alone.

While preferred embodiments of the present invention have been shown and described herein, it will be obvious to those skilled in the art that such embodiments are provided by way of example only. It is not intended that the invention be limited by the specific examples provided within the specification. While the invention has been described with reference to the aforementioned specification, the descriptions and illustrations of the embodiments herein are not meant to be construed in a limiting sense. Numerous variations, changes, and substitutions will now occur to those skilled in the art without departing from the invention. Furthermore, it shall be understood that all aspects of the invention are not limited to the specific depictions, configurations or relative proportions set forth herein which depend upon a variety of conditions and variables. It should be understood that various alternatives to the embodiments of the invention described herein may be employed in practicing the invention. It is therefore contemplated that the invention shall also cover any such alternatives, modifications, variations, or equivalents. It is intended that the following claims define the scope of the invention and that methods and structures within the scope of these claims and their equivalents be covered thereby.

### EXAMPLES

### Example 1. Super-Resolution Detection of Polypeptide Analytes

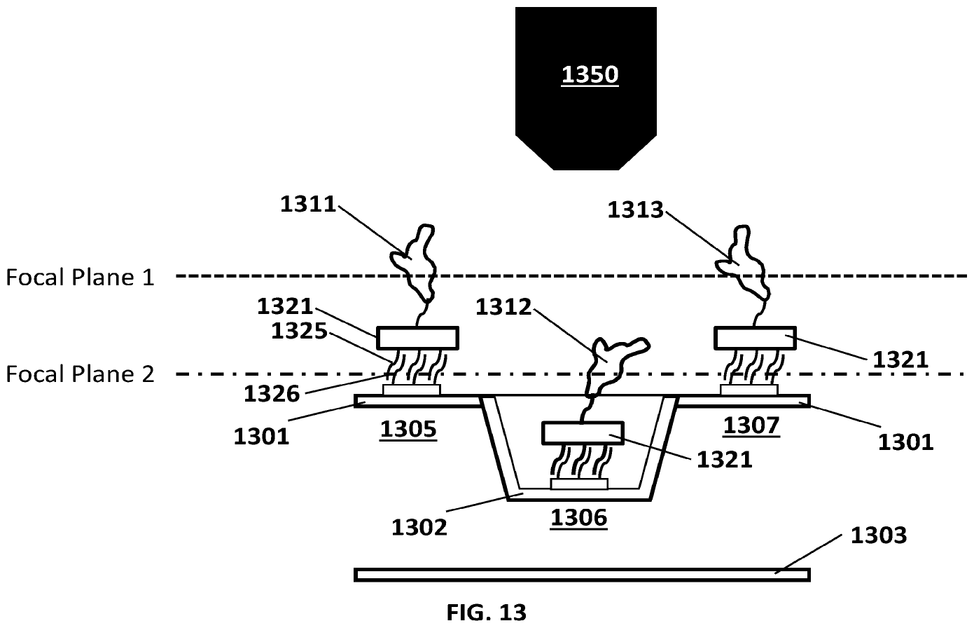

An array of analytes is provided. The array comprises a substantially planar silicon wafer that has been lithographically formed with a plurality of array sites. As shown in FIG. 13, the array comprises a first set of array sites (e.g., 1305, 1307) that are disposed at an upper level 1301 that has a substantially uniform height with respect to the underside 1303 of the silicon wafer. The array also comprises a second set of sites (e.g., 1306) that are disposed at a lower level 1302 that has a substantially uniform height with respect to the underside of the silicon wafer 1303. The average difference in height between the first set of array sites and the second set of array sites with respect to the underside 1303 of the silicon wafer is greater than a focal depth of a microscope that is used to image the array. Array site 1306 is an optically non-resolvable distance from array sites 1305 and 1307. Each array site of the first set of array sites and the second set of array sites contains a plurality of oligonucleotides 1326 that are coupled to a surface of the silicon wafer (e.g., 1301, 1302).

Analytes 1321, 1322, and 1323 are coupled to the array at array sites 1305, 1306, and 1307, respectively. Individual array sites of the first set of array sites and the second set of array sites contain one and only polypeptide of a plurality of polypeptides. Individual polypeptides are coupled to one and only one nucleic acid nanoparticle 1321. Individual nucleic acid nanoparticles 1321 comprise a plurality of pendant oligonucleotides 1325 that are complementary to the surface-coupled oligonucleotides 1326 of the plurality of array sites. Accordingly, individual analytes are coupled to one and only one array site by one and only one nucleic acid nanoparticle 1321 due to nucleic acid hybridization of the pendant oligonucleotides 1325 with the surface-coupled oligonucleotides 1326. The system further comprises a detection device 1350 that is configured to detect fluorescent signal emission from array sites. The focal plane of the detection device 1350 is adjustable between a first focal plane and a second focal plane. Signals from array sites of the upper tier of array sites (e.g., sites 1305 and 1307) will be in focus at focal plane 1. Signals from array sites of the lower tier of array sites (e.g., site 1306) will be in focus at focal plane 2.

A plurality of detectable probes is contacted to the array. Detectable probes of the plurality of detectable probes bind to analytes 1311, 1312, and 1313. The detection device 1350 is scanned across the array twice, first at focal plane 1, then at focal plane 2. Signals are collected as a function of spatial coordinate by absorption of emitted photons at pixels of a pixel array sensor. Photons are converted into electrical signals by the sensor, thereby forming measure of signal intensity as a function of spatial coordinate for both focal planes. Intensity and spatial coordinate information is provided to a processor device that performs a signal analysis process.

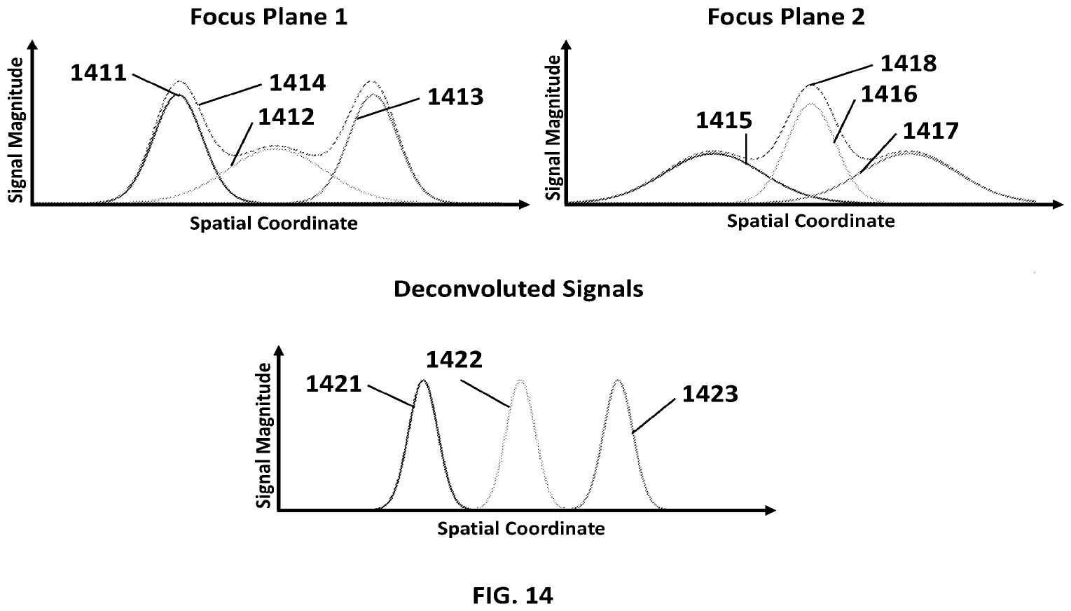

FIG. 14 shows signal intensity data as a function of spatial coordinate. The upper left plot shows signal intensity data for focal plane 1. The upper left plot shows signal intensity data for focal plane 2. The dashed lines 1414 and 1418 depict the observed signal intensities as a function of spatial coordinate for focal planes 1 and 2, respectively. Solid lines 1411, 1412, and 1413 illustrate signal deconvolution for array sites 1305, 1306, and 1307, respectively, as performed by the signal analysis process. Solid lines 1415, 1416, and 1417 illustrate signal deconvolution for array sites 1305, 1306, and 1307, respectively, as performed by the signal analysis process. The signal analysis process combines the signal intensity data from the two focal planes to obtain the lower plot of signal intensity as a function of spatial coordinate. Signals 1421, 1422, and 1423, corresponding to array sites 1305, 1306, and 1307, respectively, are obtained by the signal analysis process, thereby spatially resolving the signals from each of the three array sites.

Notwithstanding the appended claims, the disclosure set forth herein is also defined by the following clauses:
1) A method, comprising:
   a) providing an array, wherein the array comprises:
      i) a plurality of sites, wherein each site of the plurality of sites is configured to bind a single analyte;
      ii) one or more interstitial regions, wherein each site of the plurality of sites is separated by the one or more interstitial regions from each other site of the plurality of sites; and
      iii) a layer disposed on a solid support, wherein the layer comprises a first thickness at the site, and wherein the layer comprises a second thickness at the interstitial region;
   b) coupling a first single analyte to a first site of the plurality of sites and a second single analyte to a second site of the plurality of sites, wherein the first single analyte differs from the second single analyte; and
   c) detecting presence of a first signal from the first site, presence of a second signal from the second site, and absence of signal from an interstitial region of the one or more interstitial regions
   wherein an index of refraction of the solid support is greater than an index of refraction of the layer.
2) The method of clause 1, wherein providing the plurality of sites comprises forming each site of the plurality of sites on the solid support by a lithographic method.
3) The method of clause 2, further comprising disposing the layer on the solid support.
4) The method of clause 3, wherein disposing the layer on the solid support occurs before forming each site of the plurality of sites.
5) The method of clause 3, wherein disposing the layer on the solid support occurs after forming each site of the plurality of sites.
6) The method of any one of clauses 1-5, wherein the first single analyte or the second single analyte is coupled to an anchoring moiety.
7) The method of clause 6, wherein the anchoring moiety is configured to couple the first single analyte or the second single analyte to a site of the plurality of sites.
8) The method of clause 7, wherein the anchoring moiety is further configured to inhibit binding of the single analyte to the site of the plurality of sites.
9) The method of any one of clauses 6-8, wherein the anchoring moiety comprises a nanoparticle, a nucleic acid, or a polypeptide.
10) The method of clause 9, wherein the nucleic acid comprises a structured nucleic acid particle.
11) The method of clause 10, wherein the structured nucleic acid particle comprises a nucleic acid origami or a nucleic acid nanoball.
12) The method of any one of clauses 1-11, wherein the first single analyte or the second single analyte comprises a biomolecule selected from the group consisting of polypeptide, polynucleotide, polysaccharide, lipid, metabolite, pharmaceutical compound, or a combination thereof.
13) The method of clause 12, wherein the first single analyte differs from the second single analyte with respect to a difference in type of single analyte, species of single analyte, chemical property, physical property, or a combination thereof.
14) The method of clause 13, wherein the difference in physical property comprises a difference in single analyte hydrodynamic radius, single analyte length, single analyte residue sequence, single analyte mass, single analyte net electrical charge, single analyte charge density, or a combination thereof.
15) The method of any one of clauses 1-14, wherein detecting the presence of the first signal or the presence of the second signal comprises detecting the first signal or the second signal with a signal-to-noise ratio of at least 2.
16) The method of clause 15, wherein the absence of signal comprises a signal-to-noise ratio of less than 2.
17) The method of any one of clauses 1-16, wherein the detecting comprises optically detecting the presence of the first signal or the presence of the second signal.
18) The method of clause 17, wherein optical detection is performed on an optical detection system.
19) The method of clause 18, wherein the optical detection system utilizes optical microscopy, surface plasmon resonance, infrared spectroscopy, ultraviolet spectroscopy, or a combination thereof.
20) The method of any one of clauses 1-19, wherein the detecting comprises:
   i) coupling a first detectable label to the first single analyte and a second detectable label to the second single analyte; and
   ii) detecting the presence of the first signal from the first detectable label at the first site and the presence of the second signal from the second detectable label at the second site.
21) The method of clause 20, wherein the first detectable label or the second detectable label comprises an affinity agent.
22) The method of clause 20 or 21, wherein the first detectable label or the second detectable label comprises a fluorophore or a luminophore.
23) The method of any one of clauses 20-22, further comprising removing the first detectable label from the first single analyte or the second detectable label from the second single analyte.
24) The method of clause 23, wherein the removing step occurs before the detecting step.
25) The method of clause 24, wherein the removing step occurs after the detecting step.
26) The method of any one of clauses 23-25, wherein the removing step comprises a degradation reaction.
27) The method of any one of clauses 20-27, further comprising:
   i) coupling a third detectable label to the interstitial region of the one or more interstitial regions; and
   ii) detecting the absence of a third signal from the third detectable label at the interstitial region.
28) The method of any one of clause 20-27, wherein distance of the first detectable label to the layer differs from distance of the second detectable label to the layer.
29) The method of clause 27 or 28, wherein distance of the first detectable label or the second detectable label to the solid support differs from distance of the third detectable label to the solid support.
30) The method of clause 1, wherein the index of refraction of the solid support is larger than the index of refraction of the layer by at least 1.
31) The method of any one of clauses 1-30, wherein the second thickness is greater than 0.1 nanometers.
32) The method of any one of clauses 1-31, wherein the layer comprises a metal, a metal oxide, a dielectric material, or a combination thereof.
33) The method of any one of clauses 1-32, further comprising, before providing the array, determining the first thickness and the second thickness of the layer.
34) The method of clause 33, wherein the first thickness or the second thickness is determined empirically.
35) The method of clause 33 or 34, wherein the first thickness or the second thickness is determined computationally or theoretically.
36) The method of any one of clauses 1-35, wherein the layer further comprises a passivating moiety.
37) The method of clause 36, wherein the passivating moiety is configured to inhibit binding of a moiety to the layer.
38) The method of clause 36 or 37, wherein the passivating moiety is coupled to the array at a site of the plurality of sites.
39) The method of clause 36 or 37, wherein the passivating moiety is coupled to the array at the interstitial region of the one or more interstitial regions.
40) A composition, comprising:
   a) a solid support;
   b) a layer disposed upon the solid support, wherein the layer comprises raised features of a first average thickness and indented features of a second average thickness;
   c) a plurality of anchoring moieties coupled to the layer; and
   d) a plurality of single analytes, wherein each single analyte is coupled to one and only one anchoring moiety of the plurality of anchoring moieties;
   wherein an index of refraction of the solid support is greater than an index of refraction of the layer.
41) The composition of clause 40, wherein each anchoring moiety of the plurality of anchoring moieties is coupled to a single raised feature.
42) The composition of clause 40, wherein each anchoring moiety of the plurality of anchoring moieties is coupled to a single indented feature.
43) The composition of any one of clauses 40-42, wherein an anchoring moiety of the plurality of anchoring moieties is covalently coupled to the layer.
44) The composition of any one of clauses 40-42, wherein an anchoring moiety of the plurality of anchoring moieties is non-covalently coupled to the layer.
45) The composition of any one of clauses 40-44, wherein a single analyte of the plurality of single analytes is covalently coupled to an anchoring moiety of the plurality of anchoring moieties.
46) The composition of any one of clauses 40-45, wherein a single analyte of the plurality of single analytes is non-covalently coupled to an anchoring moiety of the plurality of anchoring moieties.

1. A method, comprising:
a) providing a solid support containing a first site and a second site, wherein the first site comprises a first analyte and a first immobilized avidity component, wherein the second site comprises a second analyte and a second immobilized avidity component, wherein the first immobilized avidity component differs from the second immobilized avidity component, and wherein the first site is separated from the second site by an optically non-resolvable distance;
b) coupling a first detectable probe to the first analyte at the first site, and coupling a second detectable probe to the second analyte at the second site; and
c) detecting a first signal from the first detectable probe at the first site and detecting a second signal from the second detectable probe at the second site;
wherein the first detectable probe comprises:
i) a first affinity agent that has a binding specificity for the first analyte;
ii) a first mobile avidity component that has a binding specificity for the first immobilized avidity component; and
iii) a first detectable label that is configured to produce the first signal;
and wherein the second detectable probe comprises:
i) a second affinity agent that has a binding specificity for the second analyte;
ii) a second mobile avidity component that has a binding specificity for the second immobilized avidity component; and
iii) a second detectable label that is configured to produce the second signal.

2. The method of claim 1, wherein the first site is coupled to a first anchoring moiety.

3. The method of claim 2, wherein the first anchoring moiety is further coupled to the first analyte.

4. The method of claim 3, wherein the first analyte does not contact the first site.

5. The method of claim 2, wherein the first immobilized avidity component is coupled to the first anchoring moiety.

6. The method of claim 2, wherein the first immobilized avidity component is not coupled to the first anchoring moiety.

7. The method of claim 2, wherein the first site is coupled to the first anchoring moiety by a non-covalent binding interaction.

8. The method of claim 7, wherein the non-covalent binding interaction comprises nucleic acid hybridization.

9. The method of claim 2, wherein the first site is coupled to the first anchoring moiety by a covalent binding interaction.

10. The method of claim 1, wherein the optically non-resolvable distance is less than half of the emission wavelength of the first signal or less than half the emission wavelength of the second signal.

11. The method of claim 1, wherein the optically non-resolvable distance is less than half of the excitation wavelength of the first signal or less than half the excitation wavelength of the second signal.

12. The method of claim 10, wherein the optically non-resolvable distance is no more than 400 nanometers (nm).

13. The method of claim 12, wherein the optically non-resolvable distance is no more than 200 nm.

14. The method of claim 1, wherein the solid support further comprise an interstitial region, wherein the interstitial region separates the first site from the second site.

15. The method of claim 1, wherein coupling the first detectable probe to the first analyte and coupling the second detectable probe to the second analyte comprises contacting the solid support with a fluidic medium comprising the first detectable probe and the second detectable probe.

16. The method of claim 1, wherein coupling the first detectable probe to the first analyte and coupling the second detectable probe to the second analyte comprises simultaneously performing the steps of: i) coupling the first detectable probe to the first analyte, and ii) coupling the second detectable probe to the second analyte.

17-31. (canceled)

32. The method of claim 1, wherein the first site comprises a first layer comprising a first thickness and wherein the second site comprises a second layer comprising a second thickness.

33. The method of claim 32, wherein the first thickness differs from the second thickness.

34-54. (canceled)

55. A method, comprising:
a) providing a solid support comprising a first plurality of sites and a second plurality of sites, wherein each site of the first plurality of sites has a first elevation in an optical axis, wherein each site of the second plurality of sites has a second elevation in the optical axis, wherein a plurality of analytes is coupled to the first plurality of sites and the second plurality of sites, wherein a site of the first plurality of sites is an optically non-resolvable distance from a site of the second plurality of sites in a plane that is orthogonal to the optical axis, and wherein detectable probes are coupled to analytes of the plurality of analytes;
b) detecting on a detection device signals from detectable probes, wherein the detection device comprises a single sensor, wherein the single sensor comprises a first pixel array and a second pixel array, wherein the first pixel array is spatially separated from the second pixel array on the single sensor, wherein the first pixel array is oriented at a focal point of signals from detectable probes at the first plurality of sites, and wherein the second pixel array is oriented at a focal point of signals from detectable probes at the second plurality of sites; and
c) based upon the first set of signals and the second set of signals, determining a first set of sites of the first plurality of sites containing detectable probes, and determining a second set of sites of the second plurality of sites containing detectable probes.

56-59. (canceled)

60. A system, comprising:
a) a solid support comprising a first plurality of sites and a second plurality of sites, wherein each individual site of the first plurality of sites is an optically resolvable distance from each other site of the first plurality of sites, wherein each individual site of the second plurality of sites is an optically resolvable distance from each other site of the second plurality of sites;
b) a plurality of sample analytes coupled to sites of the first plurality of sites and sites of the second plurality of sites, wherein a first site of the first plurality of sites comprises a first sample analyte of the plurality of analytes, wherein a second site of the second plurality of sites comprises a second sample analyte of the plurality of analytes, and wherein the first site and the second site are an optically non-resolvable distance apart;
c) a first detectable probe bound to the first sample analyte at the first site, and a second detectable probe bound to the second sample analyte at the second site; and
d) a detection device comprising a first pixel-based array and a second pixel-based array, wherein a first signal from the first detectable probe contacts the first pixel-based array, and wherein a second signal from the second detectable probe contacts the second pixel-based array.

61-80. (canceled)
